(19) United States
(12) Patent Application Publication
(10) Pub. No.: US 2024/0026323 A1
(43) Pub. Date: Jan. 25, 2024

(54) COMPOSITIONS AND METHODS FOR MODIFYING RNA

(71) Applicant: The Regents of the University of California, Oakland, CA (US)

(72) Inventors: Jennifer A. Doudna, Berkeley, CA (US); David Colognori, Berkeley, CA (US)

(21) Appl. No.: 18/338,150

(22) Filed: Jun. 20, 2023

Related U.S. Application Data
(60) Provisional application No. 63/354,218, filed on Jun. 21, 2022.

(51) Int. Cl. C12N 9/22; C12N 15/86

(52) U.S. Cl. CPC C12N 9/22; C12N 15/86; C12N 2310/20

(57) ABSTRACT

The present disclosure provides methods of modifying a target RNA in a eukaryotic cell. The present disclosure provides methods detecting a target RNA in a eukaryotic cell.

### CROSS-REFERENCE

This application claims the benefit of U.S. Provisional Patent Application No. 63/354,218, filed Jun. 21, 2022, which application is incorporated herein by reference in its entirety.

### STATEMENT REGARDING FEDERALLY SPONSORED RESEARCH

This invention was made with government support under Grant No. RM1HG009490 awarded by the National Institutes of Health. The government has certain rights in the invention.

### INCORPORATION BY REFERENCE OF SEQUENCE LISTING PROVIDED AS A SEQUENCE LISTING XML FILE

A Sequence Listing is provided herewith as a Sequence Listing XML, “BERK-471_SEQ_LIST.xml” created on Jun. 5, 2023 and having a size of (300,304 bytes. The contents of the Sequence Listing XML are incorporated by reference herein in their entirety.

### INTRODUCTION

Clustered Regularly Interspaced Short Palindromic Repeats (CRISPR)-Cas systems comprise a CRISPR-associated (Cas) effector polypeptide and a guide nucleic acid. Such CRISPR-Cas systems can bind to and modify a target nucleic acid. Type III CRISPR-Cas systems recognize and degrade RNA molecules using an RNA-guided mechanism that occurs widely in microbes for adaptive immunity against viruses.

RNA knockdown in eukaryotes has been accomplished by RNA interference (RNAi), an approach whereby small interfering RNAs (siRNAs) direct Argonaute nucleases to cleave complementary target RNAs. However, RNAi can cause unintended cleavage of targets carrying partial sequence complementarity, especially when this complementarity occurs within the seed region (nucleotides 2-7) of the siRNA. Furthermore, siRNAs are inefficient at targeting nuclear RNAs since the RNAi machinery is primarily localized to the cytoplasm.

There is a need in the art for RNA knockdown tools.

### SUMMARY

The present disclosure provides methods of modifying a target RNA in a eukaryotic cell. The present disclosure provides methods of detecting a target RNA in a eukaryotic cell. The present disclosure also provides compositions for carrying out such methods.

### BRIEF DESCRIPTION OF THE DRAWINGS

FIG. 1A-1L depict an all-in-one Type III CRISPR-Cas system in mammalian cells.

FIG. 2A-2F depict knockdown of endogenous nuclear and cytoplasmic RNAs.

FIG. 3A-3G depict RNA knockdown with minimal off-targets or cytotoxicity.

FIG. 4A-4D depict live-cell RNA imaging without genetic manipulation.

FIG. 5A-5E provide amino acid sequences of Streptococcus thermophilus Csm proteins. FIG. 5A: Csm1 (SEQ ID NO: 1); FIG. 5B: Csm2 (SEQ ID NO: 7); FIG. 5C: Csm3 (SEQ ID NO: 16); FIG. 5D: Csm4 (SEQ ID NO: 26); FIG. 5E: Csm5 (SEQ ID NO: 33).

FIG. 6A-6F provide amino acid sequences of Cmr proteins. FIG. 6A: Cmr1 (top to bottom: SEQ ID NOs: 40-45); FIG. 6B: Cmr2 (top to bottom: SEQ ID NOs: 46-50); FIG. 6C: Cmr3 (top to bottom: SEQ ID NOs: 51-55); FIG. 6D: Cmr4 (top to bottom: SEQ ID NOs: 56-61); FIG. 6E: Cmr5 (top to bottom: SEQ ID NOs: 62-66); FIG. 6F: Cmr6 (top to bottom: SEQ ID NOs: 67-70 and 179-180, respectively).

FIG. 7A-7E provide amino acid sequences of Csm proteins. FIG. 7A: Csm1 (top to bottom: SEQ ID NOs: 2-6); FIG. 7B: Csm2 (top to bottom: SEQ ID NOs: 8-15); FIG. 7C: Csm3 (top to bottom: SEQ ID NOs: 17-25); FIG. 7D: Csm4 (top to bottom: SEQ ID NOs: 27-32); FIG. 7E: Csm5 (top to bottom: SEQ ID NOs: 34-39).

FIG. 8A-8L depict an all-in-one Type III CRISPR-Cas system in mammalian cells. FIG. 8A, Diagram showing cis- and trans-cleavage of Cas13. FIG. 8B, Diagram showing S. thermophilus type III-A CRISPR-Cas locus. crRNAs are transcribed from the CRISPR array, processed by Cas6 and assemble with CSM proteins. FIG. 8C, Close-up of crRNA:target binding, showing the 6-nt cleavage pattern. FIG. 8D, Western blot showing proper size and expression of Cas/Csm proteins (red) in HEK293T cells. Csm1 and Csm4 are less stable when expressed separately/GAPDH (glyceraldehyde-3-phosphate dehydrogenase) shown as loading control (green). Arrows indicate faint bands. L, ladder; U, untransfected. One of two replicates with similar results is shown. FIG. 8E, Immunofluorescence showing expression and nuclear localization of Cas/Csm proteins in HEK293T cells. Scale bar, 10 μm. One of two replicates with similar results is shown. FIG. 8F, Relative GFP fluorescence (=MTI targeting crRNA/MH nontargeting crRNA) of HEK293T-GFP cells transfected with plasmids expressing Cas6, Csm1-5 and the indicated GFP-targeting crRNA, measured by flow cytometry. Error bars indicate mean±s.d. of three biological replicates. FIG. 8G, Same as f, but with the indicated Csm mutants (or crRNA Cas6 only), GFP crRNA 1 was used to target GFP. Error bars indicate mean±s.d. of three biological replicates. FIG. 8H, Same as f, but with GYP crRNA adjusted to the indicated spacer length. Error bars indicate mean±s.d. of three biological replicates. FIG. 8I, Relative GFP and RFP fluorescence of HEK293T-GFP/RFP cells transfected with plasmids expressing Cas6, Csm1-5 and the indicated crRNAs (individual or multiplexed), measured by flow cytometry. GFP crRNA 1 was used to target GFP. RFP-targeting crRNA is listed in the tables below. Error bars indicate mean±s.d. of three biological replicates. FIG. 8J, Diagram showing all-in-one delivery vector designs. FIG. 8K, Western blot showing proper size and expression of Cas/Csm proteins (red) in HEK293T cells. GAPDH is shown as loading control (green). Arrows indicate each subunit. One of two replicates with similar results is shown. FIG. 8L. Relative GFP fluorescence of HEK293T-GFP cells transfected with the indicated delivery vectors and expressing the indicated GFP-targeting crRNAs, measured by flow cytometry. Error bars indicate mean±s.d. of three biological replicates.

FIG. 9A-9G depict robust knockdown (KD) of endogenous nuclear and cytoplasmic RNAs. FIG. 9A, Relative RNA abundance (normalized to nontargeting crRNA) of the indicated targets in HEK293T cells transfected with all-in-one plasmid expressing Cas/Csm proteins and the indicated crRNAs, measured by RT-qPCR. Error bars indicate mean±s.d. of three biological replicates. FIG. 9h, Relative RNA abundance (normalized to GAPDH) of the indicated targets in untransfected. HEK293T cells, measured by RT-qPCR. Error bars indicate mean±s.d. of three biological replicates. FIG. 9c, Relative RNA abundance (normalized to nontargeting crRNA) of the indicated targets in HEK293T cells transfected with all-in-one plasmid expressing Cas/Csm proteins and the indicated crRNAs (multiplexed), measured by RT-qPCR. XIST crRNA 1, MALAT1 crRNA 1 and NEAT1 crRNA 2 were used to target XIST, MALAT1 and NEAT1, respectively. Error bars indicate mean±s.d. of three biological replicates. FIG. 9d, Relative RNA abundance (normalized to nontargeting crRNA) of XIST and BRCA1 in HEK293T cells at the indicated times post transfection with all-in-one plasmid, measured by RT-qPCR. XIST crRNA 1 and BRCA1 crRNA 2 were used to target XIST and BRCA1, respectively. Error bars indicate mean±s.d. of three biological replicates. FIG. 9e, Relative RNA abundance (normalized to nontargeting crRNA) of XIST and BRCA1 in HEK293T cells transfected with all-in-one plasmid expressing Cas/Csm proteins and intron- or exon-targeting crRNAs, measured by RT-qPCR. XIST crRNA 1 and BRCA1 crRNA 2 were used to target XIST and BRCA1 exons, respectively. Intron-targeting crRNAs are listed in the tables below. Error bars indicate mean±s.d. of three biological replicates. FIG. 9f, RNA FISH (red) for the indicated targets in HEK293T cells transfected with all-in-one plasmid expressing targeting (T) or nontargeting (NT) crRNA and RNase-active or -inactive (Mut) Cas/Csm proteins. Untransfected cells serve as internal control for transfected (green) cells. XIST crRNA 1, MALAT1 crRNA 1 and NEAT1 crRNA 2 were used to target XIST, MALAT1 and NEAT1, respectively. Scale bar, 10 μm. FIG. 9g, Quantification of f. One hundred transfected cells were counted for each condition. Error bars indicate mean±s.d. of three biological replicates.

FIG. 10A-10G depict RNA KD with minimal off-targets or cytotoxicity. FIG. 10A, FIG. 10B, Scatterplots showing differential transcript levels between HEK293T cells transfected with plasmid expressing Csm, Cas13 or shRNA targeting CKB (a) or MALAT1 (b) versus EV control. Target transcript indicated in black; off-targets (≥2-fold change) indicated in red. FIG. 10C, Quantification of upregulated or downregulated transcripts (≥2-fold change) for each sample. CKB crRNA 1, MALAT1 crRNA 2, SMARCA1 crRNA 1 and XIST crRNA 1 were used to target CKB, MALAT1, SMARCA1 and XIST, respectively, FIG. 10D, FIG. 10E, RNA-seq read coverage across target transcripts CKB (d) or MALAT1 (e). Red arrow indicates location of crRNA/shRNA target site. FIG. 10F, Relative cell viability and proliferation (normalized to EV control) of HEK293T cells at the indicated times post transfection with the indicated targeting (T) or nontargeting (NT) plasmids, measured by WST-1 assay. CKB crRNA 1 was used for targeting. Error bars indicate mean±s.d. of three biological replicates. FIG. 10G, Relative abundance of RFP-positive (transfected) HEK293T cells at the indicated times post transfection with the indicated targeting (T) or nontargeting (NT) plasmids, measured by flow cytometry. CKB crRNA 1 was used for targeting. Error bars indicate mean±s.d. of three biological replicates.

FIG. 11A-11C depict live-cell RNA imaging without genetic manipulation. FIG. 11A, Diagram showing Csm3-GFP fusion complex used for live-cell imaging. FIG. 11B, Live-cell fluorescence imaging of HEK293T cells transfected with plasmid expressing Csm3-GFP fusion complex and the indicated crRNAs (see tables below). NT, nontargeting. Scale bar, 10 μm. FIG. 11C, Quantification of b. One hundred transfected cells were counted for each condition. Error bars indicate mean±s.d. of three biological replicates.

FIG. 12A-12C Additional information regarding flow cytometry and FACS experiments. FIG. 12A, Diagram showing workflow for flow cytometry experiments. Delivery plasmids and recipient cell lines are indicated in each experiment. FIG. 12B, Diagram showing gating strategy for flow cytometry experiments. FIG. 12C, Diagram showing gating strategy for flow cytometry and FACS experiments in which transfected (RFP-positive) cells were enriched.

FIG. 13A-13C Additional information regarding RT-qPCR and RNA FISH experiments. FIG. 13A, Diagram showing workflow for RT-qPCR experiments. FIG. 13B, Diagram showing workflow for RNA FISH experiments. FIG. 13C, Diagram showing location of crRNA target site (red arrow), qPCR amplicon (solid black line), and FISH probe (dashed black line) for each transcript. Transcripts are shown 5′ to 3′, with thinner blocks representing UTR regions, thicker blocks representing coding regions, and lines representing intronic regions. Transcripts not to scale.

FIG. 14A-14G Additional information regarding RNA-sequencing experiments. FIG. 14A, Diagram showing workflow for RNA-seq experiments. FIG. 14B, Scatterplot showing differential transcript levels between HEK293T cells transfected with plasmid expressing Csm with nontargeting crRNA versus empty vector control. Up- or down-regulated transcripts (≥2-fold change) indicated in red. FIG. 14C, FIG. 14D, Scatterplots showing differential transcript levels between HEK293T cells transfected with plasmid expressing Csm, Cas13, or shRNA targeting SMARCA1 (b) or XIST (c), versus empty vector control. Target transcript indicated in black; off-targets (≥2-fold change) indicated in red. FIG. 14E, FIG. 14F, RNA-seq read coverage across target transcripts SMARCA1 (d) or XIST (e). Red arrow indicates location of crRNA/shRNA target site; EV, empty vector. FIG. 14G, Plot showing % reads with mutation compared to reference genome across the CKB locus in HEK293T cells transfected with all-in-one plasmid expressing Cas/Csm proteins and the indicated crRNAs, assayed by genomic PCR followed by DNA-seq. Red arrow indicates location of crRNA target site; NT, non-targeting.

FIG. 15A-15B Additional information regarding live-cell RNA imaging experiments. FIG. 15A, Diagram showing workflow for live-cell RNA imaging experiments. FIG. 15B, Diagram showing target-dependent activation of downstream effectors by the Csm complex.

### DEFINITIONS

The terms “polynucleotide” and “nucleic acid,” used interchangeably herein, refer to a polymeric form of nucleotides of any length, either ribonucleotides or deoxynucleotides or combinations thereof. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides.

The terms “polypeptide,” “peptide,” and “protein”, are used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence similarity can be determined in a number of different manners. To determine sequence identity, sequences can be aligned using the methods and computer programs, including BLAST, available over the world wide web at ncbi.nlm.nih.gov/BLAST. See, e.g., Altschul et al. (1990), J. Mol. Biol. 215:403-10. Another alignment algorithm is FASTA, available in the Genetics Computing Group (GCG) package, from Madison, Wisconsin, USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, California, USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate expression of a coding sequence and/or production of an encoded polypeptide in a host cell.

The term “transformation” is used interchangeably herein with “genetic modification” and refers to a permanent or transient genetic change induced in a cell following introduction of new nucleic acid (e.g., DNA exogenous to the cell) into the cell. Genetic change (“modification”) can be accomplished either by incorporation of the new nucleic acid into the genome of the host cell, or by transient or stable maintenance of the new nucleic acid as an episomal element. Where the cell is a eukaryotic cell, a permanent genetic change is generally achieved by introduction of new DNA into the genome of the cell.

“Operably linked” refers to a juxtaposition wherein the components so described are in a relationship permitting them to function in their intended manner. For instance, a promoter is operably linked to a coding sequence if the promoter affects its transcription or expression. As used herein, the terms “heterologous promoter” and “heterologous control regions” refer to promoters and other control regions that are not normally associated with a particular nucleic acid in nature. For example, a “transcriptional control region heterologous to a coding region” is a transcriptional control region that is not normally associated with the coding region in nature.

As used herein, the term “guide RNA” (gRNA) and the like refer to an RNA that guides a Type III CRISPR-Cas effector polypeptide (or a fusion protein comprising a Type III CRISPR-Cas effector polypeptide) to a target sequence in a target RNA.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a Csm polypeptide” includes a plurality of such polypeptides and reference to “the RNA molecule” includes reference to one or more RNA molecules and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The use of the terms “a,” “an,” and “the,” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context.

As used herein, the term “about” used in connection with an amount indicates that the amount can vary by 10% of the stated amount. For example, “about 100” means an amount of from 90-110. Where about is used in the context of a range, the “about” used in reference to the lower amount of the range means that the lower amount includes an amount that is 10% lower than the lower amount of the range, and “about” used in reference to the higher amount of the range means that the higher amount includes an amount 10% higher than the higher amount of the range. For example, from about 100 to about 1000 means that the range extends from 90 to 1100.

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

It is understood that aspects and embodiments of the present disclosure described herein include “comprising,” “consisting,” and “consisting essentially of” aspects and embodiments.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides methods of modifying a target RNA in a eukaryotic cell. The present disclosure provides methods of detecting a target RNA in a eukaryotic cell. The present disclosure also provides compositions for carrying out such methods.

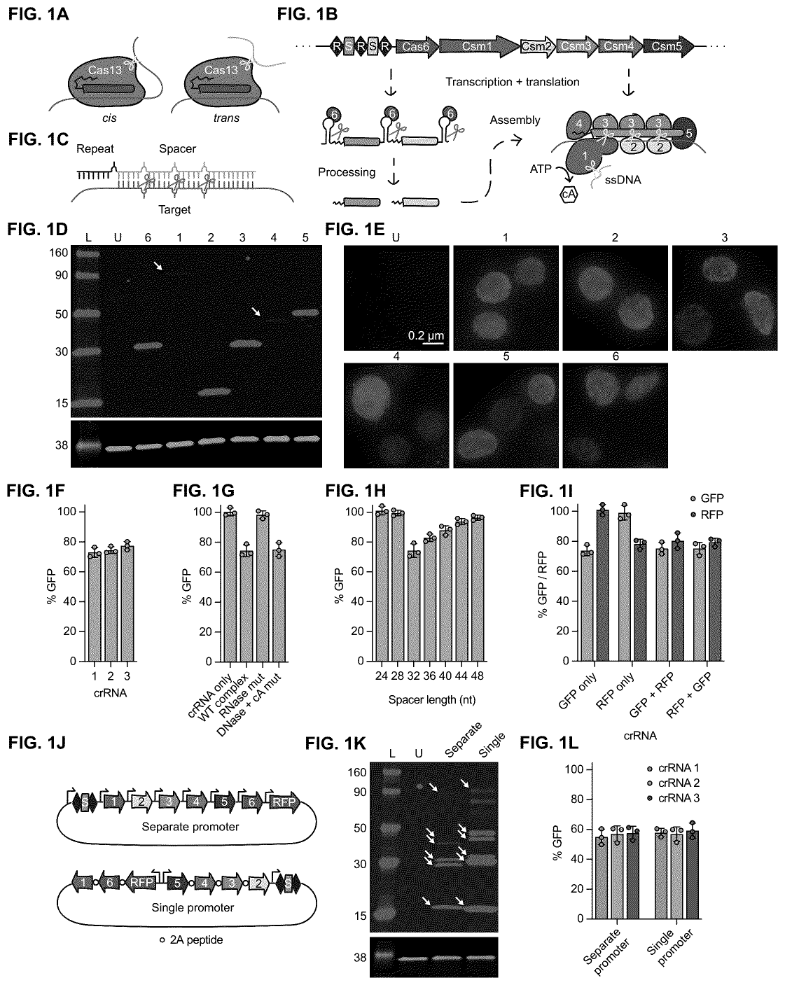

The natural multiprotein Csm complex comprises five subunits (Csm1-5) in varying stoichiometries and relies on an additional protein, Cas6, for processing the precursor crRNA (FIG. 1b). The crRNA lies at the core of the complex, with Csm1 and Csm4 binding the end, Csm5 binding the 3′ end and multiple copies of Csm2 and Csm3 wrapping around the center. The complex contains a groove along its length into which target RNAs can enter and hybridize to the variable spacer region of the crRNA. Csm1 and Csm4 specifically recognize the region of the crRNA derived from the CRISPR repeat. Each Csm3 subunit has ribonuclease (RNase) activity, leading to multiple cleavage sites within the target RNA spaced six nucleotides (nt) apart (FIG. 1c). Csm1 functions as a nonspecific single-stranded DNase (ssDNase) and a cyclic oligoadenylate (cA) synthase (FIG. 1b). The ssDNase activity is thought to defend against actively transcribed (R-looped) or ssDNA foreign genomes, while the latter acts as a second messenger that activates downstream effectors in trans, such as the RNase Csm6. Notably, all three catalytic activities are performed by independent domains of the Csm complex and can be individually ablated.

As shown the working examples below, Csm is an attractive RNA knockdown (KD) tool over current methods. A self-contained system found only in prokaryotes, it can be orthogonally introduced into eukaryotes without intersecting host RNA regulatory pathways. Furthermore, unlike RNAi, it can be localized to the nucleus and used to target nuclear noncoding RNAs and pre-mRNAs. Compared to Cas13, Csm cleaves only in cis within the crRNA:target complementary region and thus does not suffer from trans-cleavage activity. Additionally, unlike Cas13, Csm-mediated RNA cleavage does not preferentially occur at a particular nt base (for example, U) nor is directly influenced by sequence flanking the target (for example, tag:antitag complementarity).

### Methods of Modifying a Target RNA

The present disclosure provides methods of modifying a target RNA in a eukaryotic cell. The methods comprise introducing into the eukaryotic cell: a) one or more nucleic acids comprising nucleotide sequences encoding a multi-subunit Type III CRISPR-Cas effector polypeptide, wherein the multi-subunit Type III CRISPR-Cas effector polypeptide comprises at least 5 subunits; and b) one or more guide RNAs, wherein each of the one or more guide RNAs comprises: i) a targeting region that comprises a nucleotide sequence that is complementary to a target sequence in the target RNA; and ii) a protein-binding region that binds to the multi-subunit Type III CRISPR-Cas effector polypeptide; or a nucleic acid comprising a nucleotide sequence encoding the guide RNA, wherein the multi-subunit Type III CRISPR-Cas effector polypeptide is produced in the cell and forms a complex with the guide RNA, and wherein the complex binds to the target RNA and results in modification of the target RNA in the cell.

In some cases, the one or more nucleic acids are or are present in one or more recombinant expression vectors. Examples of suitable recombinant expression vectors include a recombinant adeno-associated virus vector, a recombinant lentivirus vector, a recombinant adenovirus vector, and a recombinant retroviral vector.

In some cases, the nucleotide sequences encoding the at least 5 subunits are operably linked to a single promoter. In some cases, the nucleotide sequences encoding the at least 5 subunits are operably linked to two or more different promoters. Thus, e.g., in some cases, nucleotide sequences encoding the at least 5 subunits are each operably linked to a different promoter. In some cases, a first promoter is operably linked to nucleotide sequences encoding 2 of the at least 5 subunits; and a second promoter is operably linked to nucleotide sequences encoding the other 3 of the at least 5 subunits.

In some cases, the promoter is a constitutively active promoter. In some cases, the promoter is a regulatable promoter. In some cases, the promoter is an inducible promoter. In some cases, the promoter is a tissue-specific promoter. In some cases, the promoter is a cell type-specific promoter. In some cases, the transcriptional control element (e.g., the promoter) is functional in a targeted cell type or targeted cell population.

In some cases, the one or more nucleic acids comprising nucleotide sequences encoding the multi-subunit Type III CRISPR-Cas effector polypeptide comprise a nucleotide sequence encoding the guide RNA.

As noted above, the target RNA is present in a eukaryotic cell. In some cases, the target RNA is present in the nucleus or in an organelle (e.g., in a mitochondrion). In some cases, the target RNA is present in the cytoplasm of the eukaryotic cell.

Suitable eukaryotic cells include, e.g., a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, angiosperms, ferns, clubmosses, hornworts, liverworts, mosses, dicotyledons, monocotyledons, etc.), an algal cell, (e.g., Botryococcus braunii, Chlamydomonas reinhardtii, Nannochloropsis gaditana, Chlorella pyrenoidosa, Sargassum patens, C. agardh, and the like), seaweeds (e.g. kelp) a fungal cell (e.g., a yeast cell, a cell from a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., an ungulate (e.g., a pig, a cow, a goat, a sheep); a rodent (e.g., a rat, a mouse); a non-human primate; a human; a feline (e.g., a cat); a canine (e.g., a dog); etc.), and the like. In some cases, the cell is a cell that does not originate from a natural organism (e.g., the cell can be a synthetically made cell; also referred to as an artificial cell). In some cases, the eukaryotic cell is a mammalian cell, a plant cell, an insect cell, a reptile cell, an amphibian cell, a protozoan cell, an arachnid cell, an avian cell, or a fish cell.

In some cases, the cell is in vitro. In some cases, the cell is in vivo. Thus, in some cases, the eukaryotic cell is a eukaryotic cell present in a mammal (e.g., a human, a non-human mammal, etc.), a plant, a reptile, an amphibian, a bird, an insect, an arachnid, a fish, etc.

The target RNA can be any RNA in a eukaryotic cell. In some cases, the target RNA is a coding RNA. In some cases, the coding RNA is an mRNA or a pre-mRNA. In some cases, the target RNA is a non-coding RNA. In some cases, the target RNA is a regulatory RNA. In some cases, the non-coding RNA is a transfer RNA (tRNA), a pre-ribosomal RNA, a ribosomal RNA (rRNA), a microRNA (miRNA), an enhancer RNA (eRNA), a Piwi-interacting RNA (piRNA), a small nucleolar RNA (snoRNA), a small nuclear RNA (snRNA), a small interfering RNA (siRNA), a circRNA, or a long non-coding RNA (lncRNA). In some cases, the target RNA is an endogenous RNA. In some cases, the target RNA is mitochondrial RNA or chloroplast RNA. In some cases, the target RNA is an exogenous RNA. In some cases, the exogenous RNA is a viral RNA.

Modification of a target RNA will in some cases comprise cleavage of the target RNA. In some cases, cleavage of the target RNA reduces the level of the target RNA in the cell, compared to the level of the target RNA in a cell not treated with a method of the present disclosure. For example, in some cases, carrying out a method of the present disclosure on a eukaryotic cell reduces the level of a target RNA in the cell by at least 10%, at least 15%, at least 20%, at least 25%, at least 30%, at least 40%, at least 50%, at least 60%, at least 70%, at least 80%, at least 90%, or more than 90%, compared to the level of the target RNA in a control cell not treated with the method.

In some cases, modification of the target RNA comprises methylation of the target RNA. In some cases, modification of the target RNA comprises acetylation. In some cases, modification of the target RNA comprises adenylation. For example, in some cases, one or more of the subunits of the Type III CRISPR-Cas effector polypeptide is a fusion protein comprising the subunit and a heterologous fusion partner, where the heterologous fusion partner has an activity, such as methylase activity, that modifies the target RNA.

The multi-subunit Type III CRISPR-Cas effector polypeptide will in some cases be a Type IIIA CRISPR-Cas effector polypeptide comprising Cas10/Csm1, Csm2, Csm3, Csm4, and Csm5 polypeptides. The multi-subunit Type III CRISPR-Cas effector polypeptide will in some cases be a Type IIIB CRISPR-Cas effector polypeptide comprising Cmr1, Cmr2, Cmr3, Cmr4, Cmr5, and Cmr6 subunits.

In some cases, the multi-subunit Type III CRISPR-Cas effector polypeptide comprises one or more amino acid substitutions that reduce DNAse activity. In some cases, the one or more amino acid substitutions that reduce DNAse activity comprise a substitution of H15 (e.g., H15A), a substitution of D16 (e.g., D16A), or both H15 and D16 (e.g., H15A/D16A), of a Csm1 polypeptide. In some cases, the multi-subunit Type III CRISPR-Cas effector polypeptide comprises one or more amino acid substitutions that reduce polymerization of ATP into a cyclic oligoadenylate (cA) molecule. In some cases, the one or more amino acid substitutions that reduce polymerization of ATP to cA comprise a substitution of D577 (e.g., D577A), a substitution of D578 (e.g., D578A), or a substitution of both D577 and D578 (e.g., D577A/D578A) of a Csm10/Csm1 polypeptide.

### Csm Proteins

The multi-subunit Type III CRISPR-Cas effector polypeptide will in some cases, be a Type IIIA CRISPR-Cas effector polypeptide comprising Cas10/Csm1, Csm2, Csm3, Csm4, and Csm5 polypeptides.

The multi-subunit Csm complex is a Type III-A RNA-targeting Cas effector consisting of 5 subunits (Csm1-5) in varying stoichiometries, which also relies on Cas6 for processing the mature crRNA. The crRNA lies at the core of the complex, with Csm1 and Csm4 binding the 5′ end, Csm5 binding the 3′ end, and multiple copies of Csm2 and Csm3 wrapping around the center. The complex contains a groove along its length into which target RNAs can enter and hybridize to the variable spacer region of the crRNA. Csm1 and Csm4 specifically recognize the 5′ region of the crRNA derived from the CRISPR repeat. Each Csm3 subunit has RNase activity, leading to multiple cleavage sites within the target RNA spaced 6 nucleotides apart (FIG. 1C). Csm1 also contains two catalytic activities: 1. non-specific ssDNase activity; and 2. polymerization of ATP into a cyclic oligoadenylate (cA) molecule. The former is thought to defend against ssDNA or actively transcribed (R-looped) foreign genomes, while the latter acts as a second messenger that activates downstream defense effectors in trans, such as the RNase Csm6. All three catalytic activities are carried out by independent domains of the Csm complex and can be individually ablated.

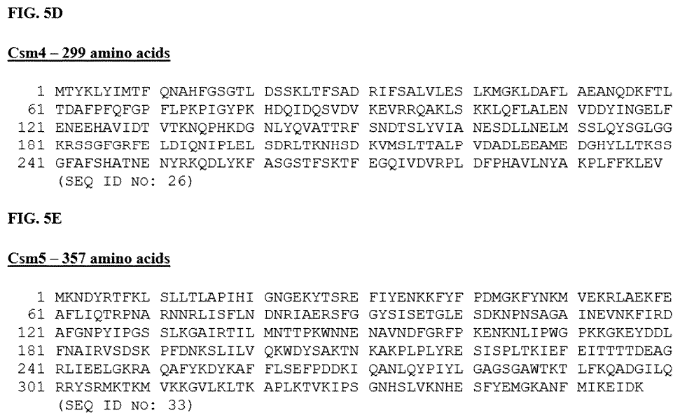

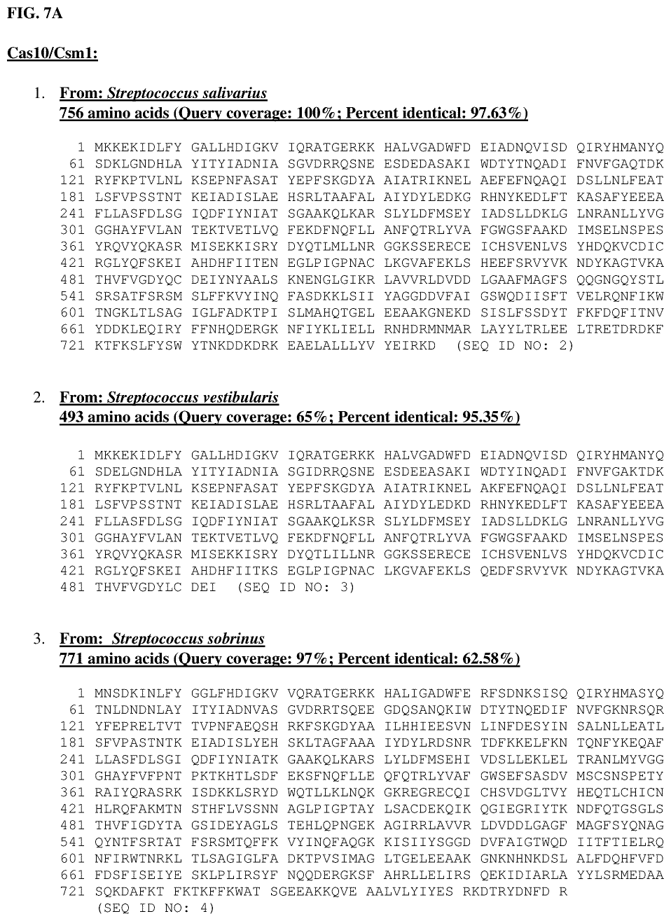

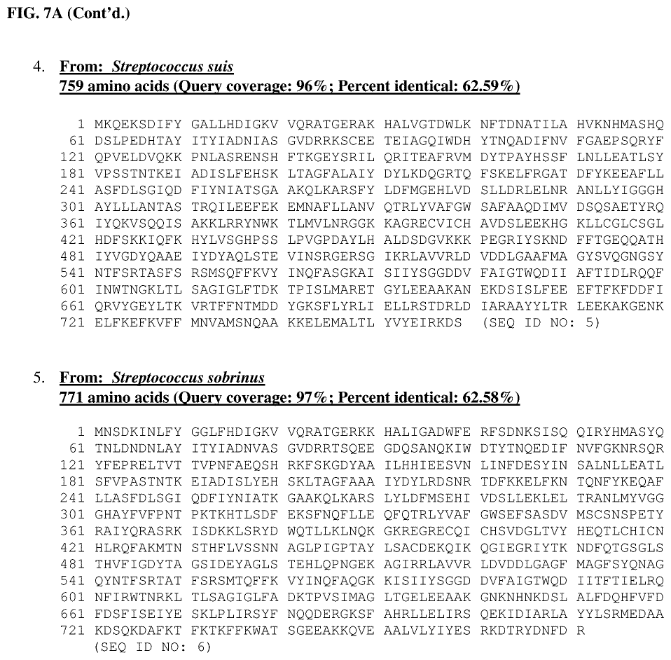

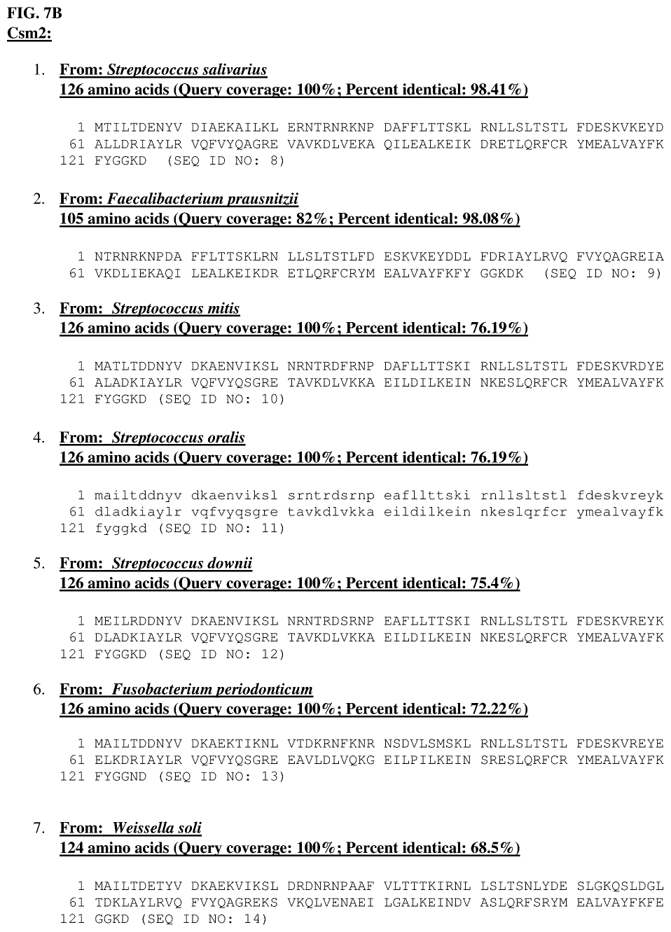

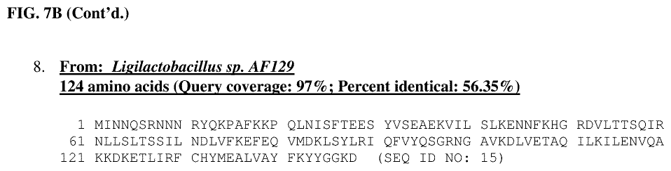

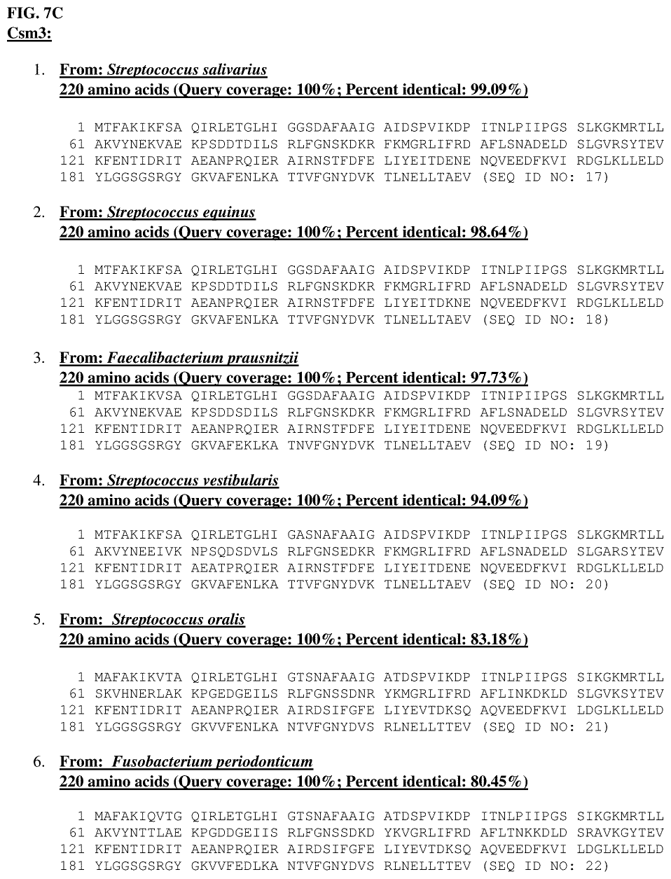

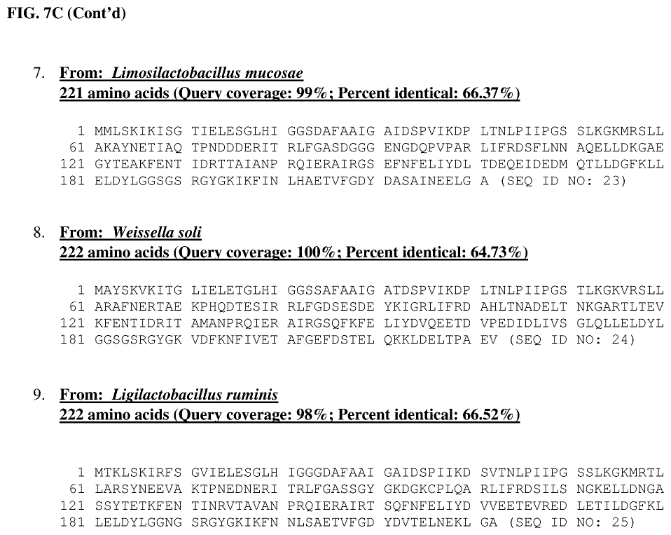

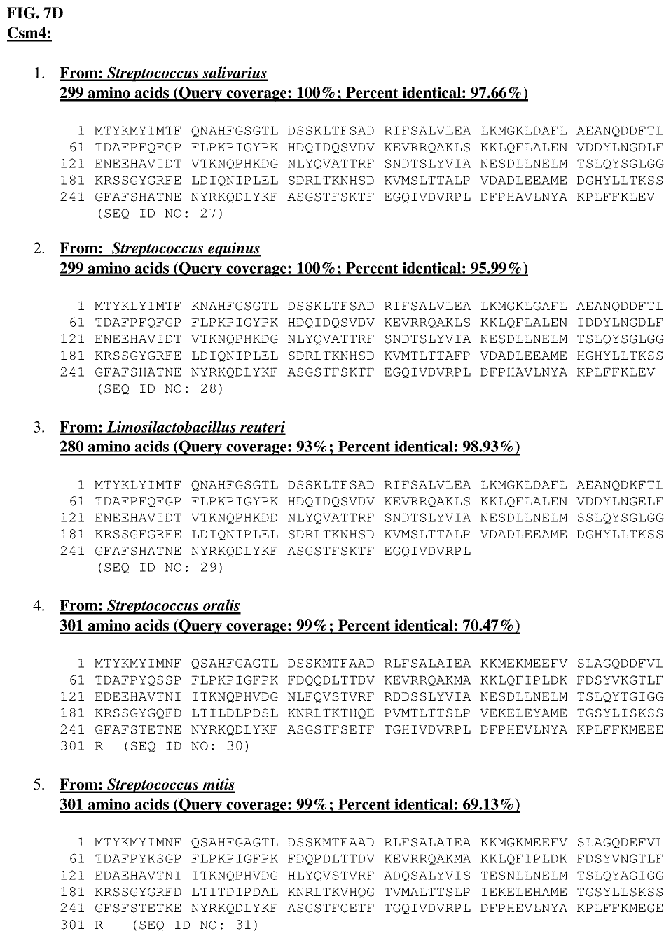

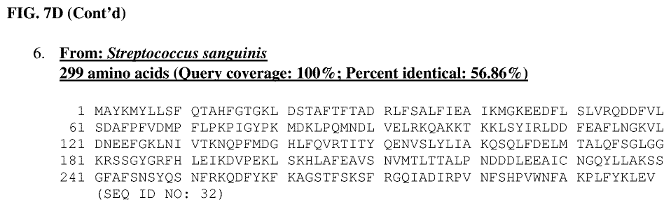

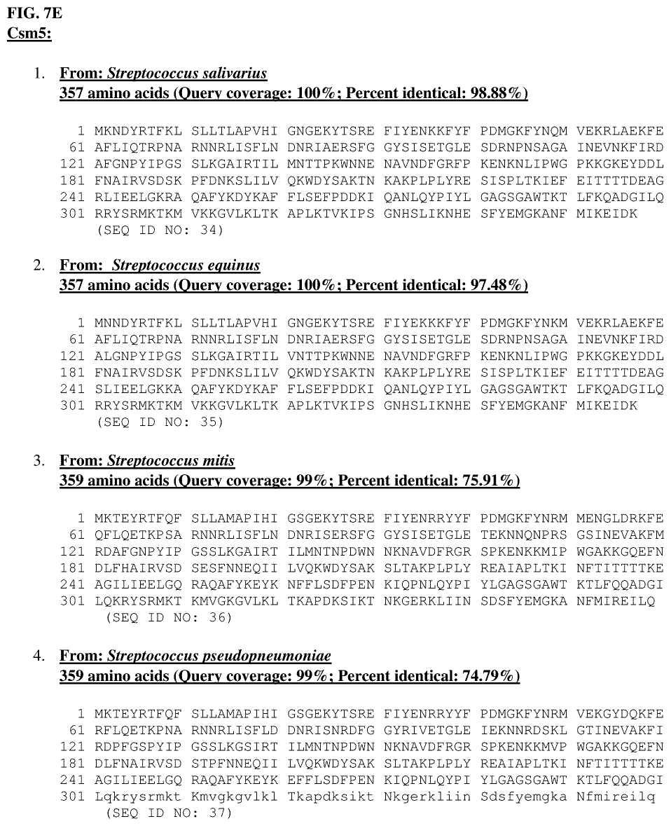

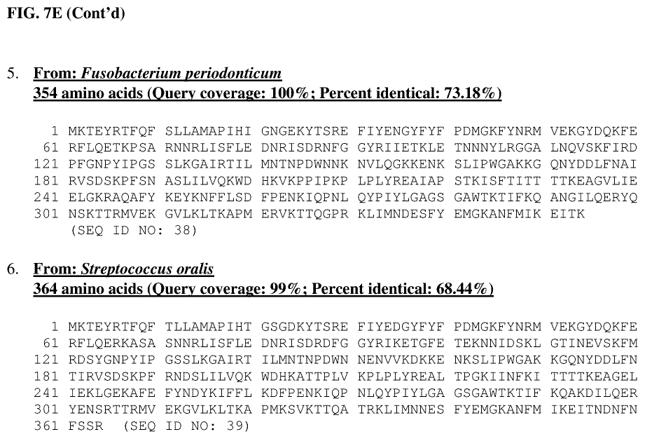

In some cases, the Cas10/Csm1, Csm2, Csm3, Csm4, and Csm5 polypeptides each independently comprise an amino acid sequence having at least 50%, at least 60%, at least 70%, at least 75%, at least 80%, at least 85%, at least 90%, at least 95%, or 100%, amino acid sequence identity to the amino acid sequences of the Cas10/Csm1, Csm2, Csm3, Csm4, and Csm5 polypeptides depicted in FIG. 5A-5E (SEQ ID Nos: 1, 7, 16, 26, and 33, respectively). In some cases, the Cas10/Csm1, Csm2, Csm3, Csm4, and Csm5 polypeptides each independently comprise an amino acid sequence having at least 50%, at least 60%, at least 70%, at least 75%, at least 80%, at least 85%, at least 90%, at least 95%, or 100%, amino acid sequence identity to any of the amino acid sequences of the Cas10/Csm1, Csm2, Csm3, Csm4, and Csm5 polypeptides depicted in FIG. 7A-7E (SEQ ID Nos: 2-6 (Csm1), 8-15 (Csm2), 17-25 (Csm3), 27-32 (Csm4), and 34-39 (Csm5)).

In some cases, the multi-subunit Type III CRISPR-Cas effector polypeptide comprises one or more amino acid substitutions that reduce DNAse activity. In some cases, the one or more amino acid substitutions that reduce DNAse activity comprise a substitution of H15 (e.g., H15A), a substitution of D16 (e.g., D16A), or both H15 and D16 (e.g., H15A/D16A), of a Csm1 polypeptide.

In some cases, the multi-subunit Type III CRISPR-Cas effector polypeptide comprises one or more amino acid substitutions that reduce polymerization of ATP into a cyclic oligoadenylate (cA) molecule. In some cases, the one or more amino acid substitutions that reduce polymerization of ATP to cA comprise a substitution of D577 (e.g., D577A), a substitution of D578 (e.g., D578A), or a substitution of both D577 and D578 (e.g., D577A/D578A) of a Csm10/Csm1 polypeptide.

In some cases, the multi-subunit Type III CRISPR-Cas effector polypeptide comprises one or more amino acid substitutions that reduce RNase activity. In some cases, the one or more amino acid substitutions that reduce RNase activity include a D33 (e.g., D33A) substitution of a Csm3 polypeptide. Such a polypeptide lacks RNase activity and instead only binds to the target RNA. For example, in some cases, the RNA cleaving protein has a mutation at a position corresponding to D33 (e.g., D33A) of the Csm3 protein sequence of SEQ ID NO: 16. As such, in some cases, the multi-subunit Type III CRISPR-Cas effector polypeptide includes a Csm3 protein having a mutation at position D33 (e.g., D33A).

As such, in some cases the Cas10/Csm1 polypeptide comprises an amino acid sequence having at least 50% (e.g., at least 60%, 70%, 75%, 80%, 85%, 90%, 95%, 97%, 98%, 99%, or 99.5%, or 100%) amino acid sequence identity to the amino acid sequence of any one of SEQ ID Nos: 1-6 (and in some such cases the Csm1 polypeptide is a variant with reduced DNAse activity and/or reduced reduce ATP polymerization activity (into cA) as discussed above). In some cases, the Cas10/Csm1 polypeptide comprises an amino acid sequence having at least 80% (e.g., at least 85%, 90%, 95%, 97%, 98%, 99%, or 99.5%, or 100%) amino acid sequence identity to the amino acid sequence of any one of SEQ ID Nos: 1-6 (and in some such cases the Csm1 polypeptide is a variant with reduced DNAse activity and/or reduced reduce ATP polymerization activity (into cA) as discussed above). In some cases, the Cas10/Csm1 polypeptide comprises an amino acid sequence having the amino acid sequence of any one of SEQ ID Nos: 1-6 (and in some such cases the Csm1 polypeptide is a variant with reduced DNAse activity and/or reduced reduce ATP polymerization activity (into cA) as discussed above).

In some cases the Csm2 polypeptide comprises an amino acid sequence having at least 50% (e.g., at least 60%, 70%, 75%, 80%, 85%, 90%, 95%, 97%, 98%, 99%, or 99.5%, or 100%) amino acid sequence identity to the amino acid sequence of any one of SEQ ID Nos: 7-15. In some cases, the Csm2 polypeptide comprises an amino acid sequence having at least 80% (e.g., at least 85%, 90%, 95%, 97%, 98%, 99%, or 99.5%, or 100%) amino acid sequence identity to the amino acid sequence of any one of SEQ ID Nos: 7-15. In some cases, the Csm2 polypeptide comprises an amino acid sequence having the amino acid sequence of any one of SEQ ID Nos: 7-15.

In some cases the Csm3 polypeptide comprises an amino acid sequence having at least 50% (e.g., at least 60%, 70%, 75%, 80%, 85%, 90%, 95%, 97%, 98%, 99%, or 99.5%, or 100%) amino acid sequence identity to the amino acid sequence of any one of SEQ ID Nos: 16-25 (and in some such cases the Csm3 polypeptide is a variant with ablated RNase activity, e.g., includes a mutation at a position corresponding to D33 (e.g., D33A) of SEQ ID NO: 16 as discussed above). In some cases, the Csm3 polypeptide comprises an amino acid sequence having at least 80% (e.g., at least 85%, 90%, 95%, 97%, 98%, 99%, or 99.5%, or 100%) amino acid sequence identity to the amino acid sequence of any one of SEQ ID Nos: 16-25 (and in some such cases the Csm3 polypeptide is a variant with ablated RNase activity, e.g., includes a mutation at a position corresponding to D33 (e.g., D33A) of SEQ ID NO: 16 as discussed above). In some cases, the Csm3 polypeptide comprises an amino acid sequence having the amino acid sequence of any one of SEQ ID Nos: 16-25 (and in some such cases the Csm3 polypeptide is a variant with ablated RNase activity, e.g., includes a mutation at a position corresponding to D33 (e.g., D33A) of SEQ ID NO: 16 as discussed above).

In some cases the Csm4 polypeptide comprises an amino acid sequence having at least 50% (e.g., at least 60%, 70%, 75%, 80%, 85%, 90%, 95%, 97%, 98%, 99%, or 99.5%, or 100%) amino acid sequence identity to the amino acid sequence of any one of SEQ ID Nos: 26-32. In some cases, the Csm4 polypeptide comprises an amino acid sequence having at least 80% (e.g., at least 85%, 90%, 95%, 97%, 98%, 99%, or 99.5%, or 100%) amino acid sequence identity to the amino acid sequence of any one of SEQ ID Nos: 26-32. In some cases, the Csm4 polypeptide comprises an amino acid sequence having the amino acid sequence of any one of SEQ ID Nos: 26-32.

In some cases the Csm5 polypeptide comprises an amino acid sequence having at least 50% (e.g., at least 60%, 70%, 75%, 80%, 85%, 90%, 95%, 97%, 98%, 99%, or 99.5%, or 100%) amino acid sequence identity to the amino acid sequence of any one of SEQ ID Nos: 33-39. In some cases, the Csm5 polypeptide comprises an amino acid sequence having at least 80% (e.g., at least 85%, 90%, 95%, 97%, 98%, 99%, or 99.5%, or 100%) amino acid sequence identity to the amino acid sequence of any one of SEQ ID Nos: 33-39. In some cases, the Csm5 polypeptide comprises an amino acid sequence having the amino acid sequence of any one of SEQ ID Nos: 33-39.

### Cmr Proteins

The multi-subunit Type III CRISPR-Cas effector polypeptide will in some cases be a Type IIIB CRISPR-Cas effector polypeptide comprising Cmr1, Cmr2, Cmr3, Cmr4, Cmr5, and Cmr6 subunits.

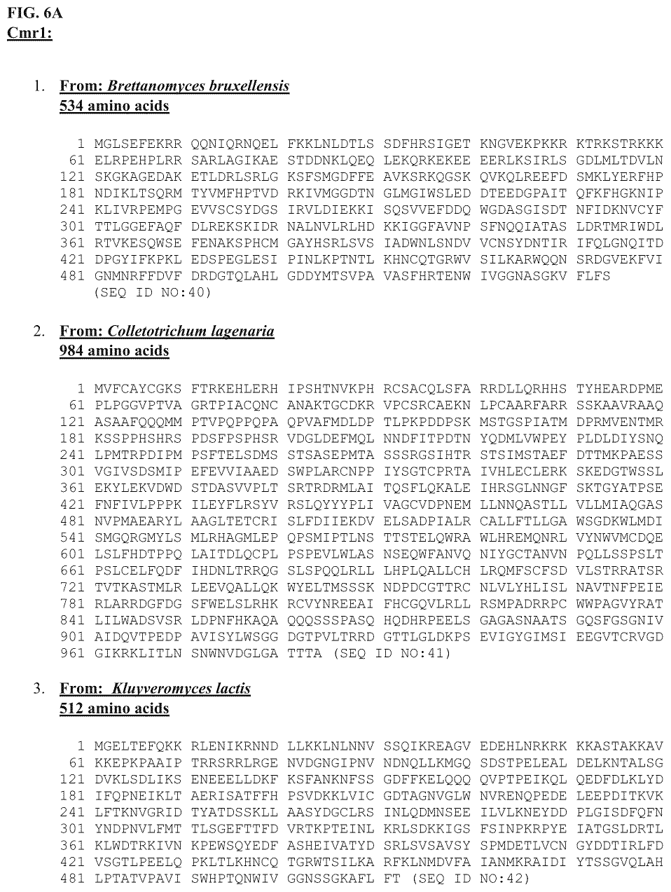

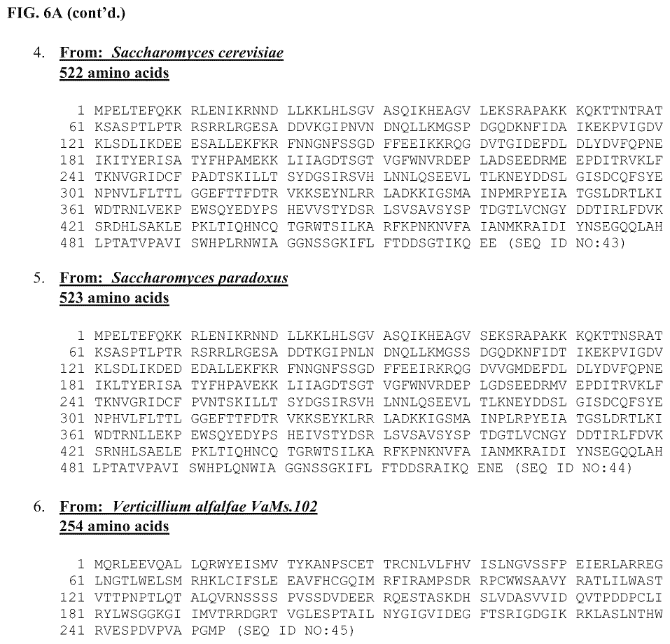

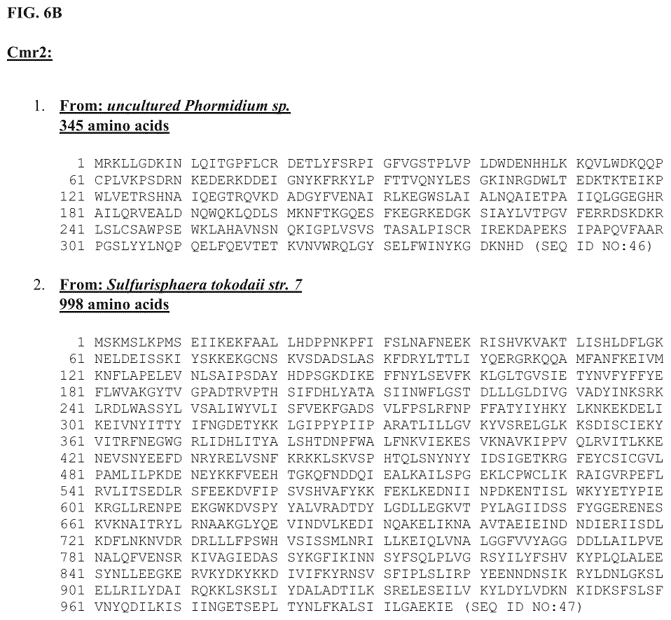

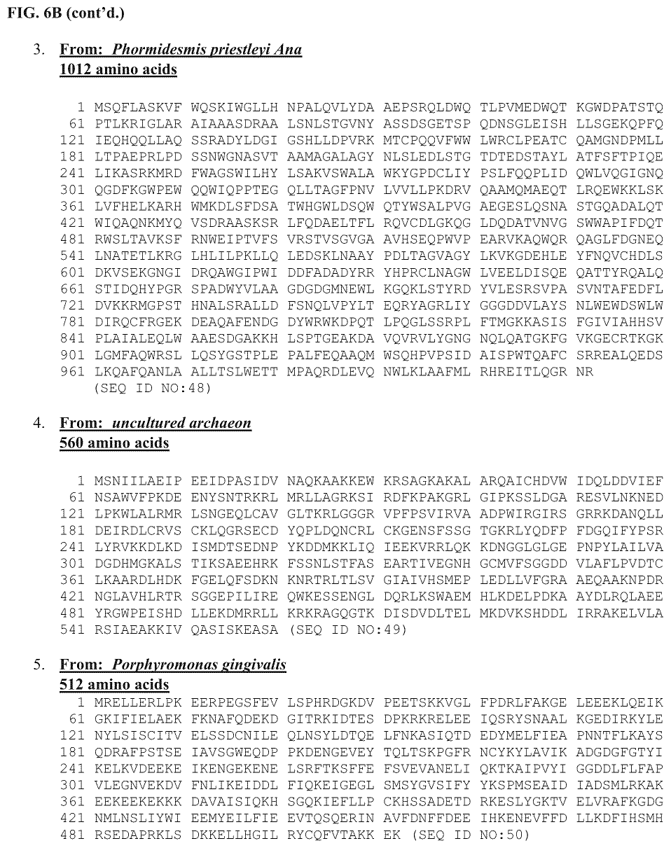

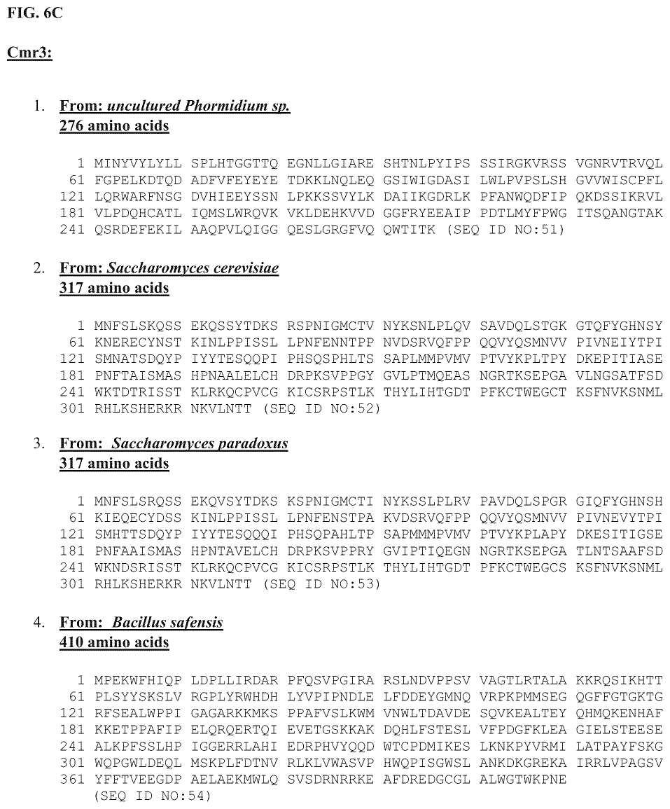

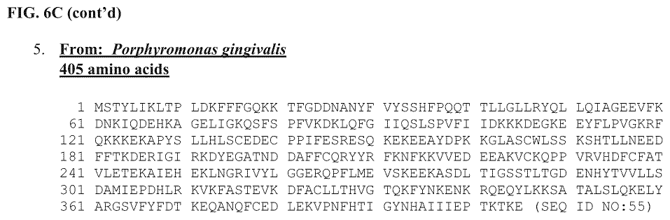

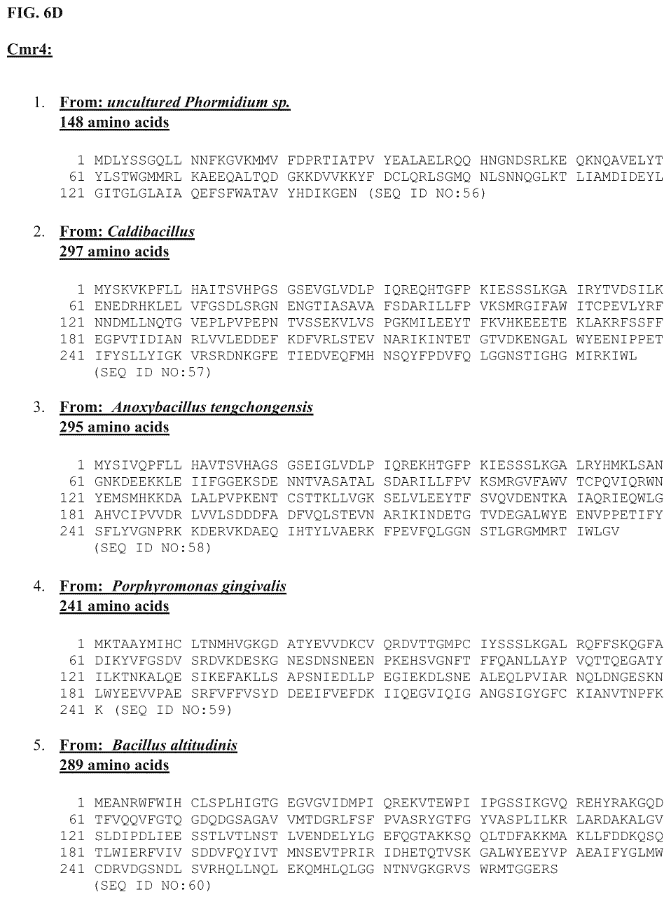

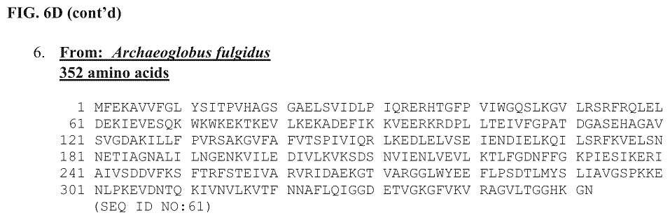

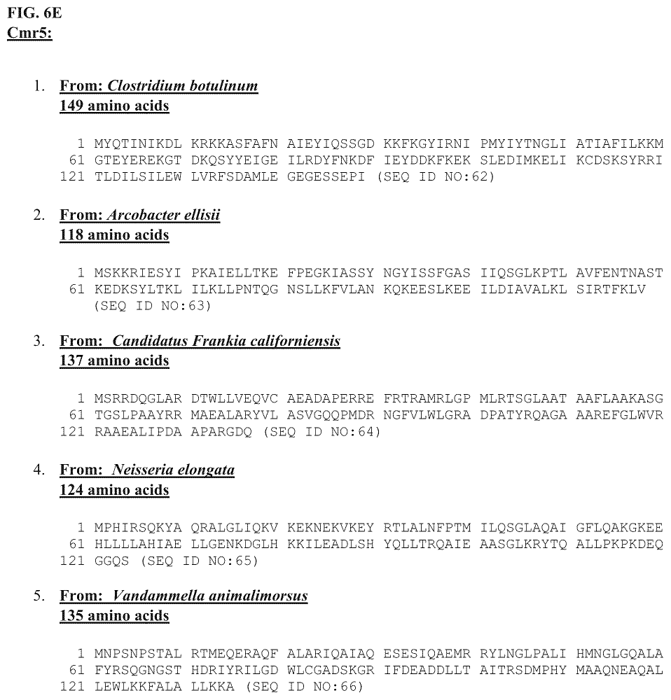

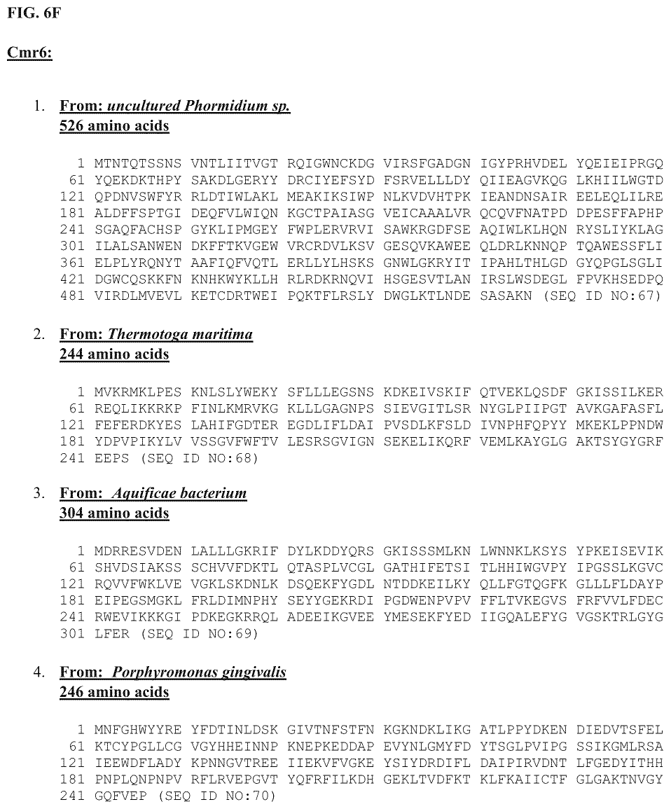

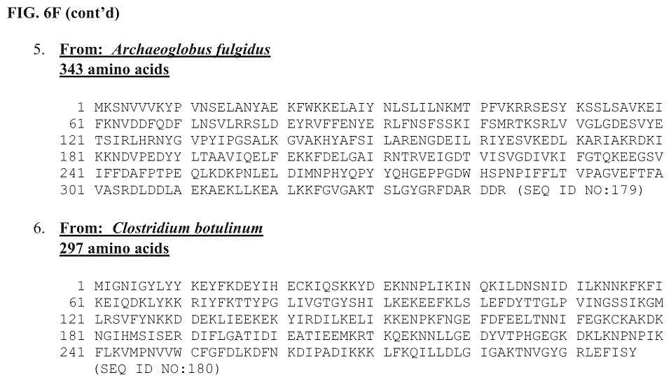

In some cases, the Cmr1, Cmr2, Cmr3, Cmr4, Cmr5, and Cmr6 polypeptides each independently comprise an amino acid sequence having at least 50%, at least 60%, at least 70%, at least 75%, at least 80%, at least 85%, at least 90%, at least 95%, or 100%, amino acid sequence identity to any of the amino acid sequences of the Cmr1, Cmr2, Cmr3, Cmr4, Cmr5, and Cmr6 polypeptides depicted in FIG. 6A-6E (SEQ ID Nos: 40-45 (Cmr1), 46-50 (Cmr2), 51-55 (Cmr3), 56-61 (Cmr4), 62-66 (Cmr5), and 67-70 and 179-180 (Cmr6)).

In some cases, a nucleotide sequence encoding a Csm or a Cmr polypeptide is codon optimized. This type of optimization can entail a mutation of a Csm polypeptide-encoding or a Cmr polypeptide-encoding nucleotide sequence to mimic the codon preferences of the intended host organism or cell while encoding the same protein. Thus, the codons can be changed, but the encoded protein remains unchanged. For example, if the intended target cell was a human cell, a human codon-optimized Csm- or Cmr-encoding nucleotide sequence could be used. As another non-limiting example, if the intended host cell were a mouse cell, then a mouse codon-optimized Csm- or Cmr-encoding nucleotide sequence could be generated. As another non-limiting example, if the intended host cell were a plant cell, then a plant codon-optimized Csm- or Cmr-encoding nucleotide sequence could be generated. As another non-limiting example, if the intended host cell were an insect cell, then an insect codon-optimized Csm- or Cmr-encoding nucleotide sequence could be generated.

Codon usage tables are readily available, for example, at the “Codon Usage Database” available at www[dot]kazusa[dot]or[dot]jp[forwardslash]codon. In some cases, a nucleic acid of the present disclosure comprises a Csm polypeptide-encoding or a Cmr polypeptide-encoding nucleotide sequence that is codon optimized for expression in a eukaryotic cell. In some cases, a nucleic acid of the present disclosure comprises a Csm polypeptide-encoding or a Cmr polypeptide-encoding nucleotide sequence that is codon optimized for expression in an animal cell. In some cases, a nucleic acid of the present disclosure comprises a Csm polypeptide-encoding or a Cmr polypeptide nucleotide sequence that is codon optimized for expression in a fungus cell. In some cases, a nucleic acid of the present disclosure comprises a Csm polypeptide-encoding or a Cmr polypeptide-encoding nucleotide sequence that is codon optimized for expression in a plant cell.

### Guide RNAs

Certain aspects of the present disclosure relate to guide RNAs and their use in CRISPR-based targeting of a target nucleic acid. Guide RNAs of the present disclosure are capable of binding or otherwise interacting with a subject multi-subunit Type III CRISPR-Cas effector polypeptide to facilitate targeting to a target nucleic acid. Suitable and exemplary guide RNAs are provided herein and design of such to target a particular nucleic acid will be readily apparent to one of skill in the art.

A guide RNA can be said to include two segments, a targeting segment and a protein-binding segment. The targeting segment of a guide RNA includes a nucleotide sequence (a guide sequence) that is complementary to (and therefore hybridizes with) a specific sequence (a target site) within a target nucleic acid. The protein-binding segment—located 5′ of the targeting segment—is also referred to herein as the “constant region” or “handle” of the guide RNA, e.g., “a 5′ handle”. The protein-binding segment (or “protein-binding sequence”) interacts with (binds to) a subject multi-subunit Type III CRISPR-Cas effector polypeptide.

Type IIIA and IIIB protein-binding segment sequences are known in the art, and are also referred to as a “handle”, a “5′ handle”, and a “8nt 5′ handle,” and one of ordinary skill in the art would be able to readily identify an appropriate sequence for any desired Type IIIA or IIIB system. For example, as would be known to one of ordinary skill in the art, the 8 nucleotide 5′ handle from the type IIIA system of S. thermophilus is ACGGAAAC, from T. onnurineus is GUGGAAAG, and from S. sollataricuv is ATTGAAAG, while the 8 nucleotide 5′ handle from the type IIIB system of T. thermophilus is ATTGAAAC—and standard methods can be employed to identify a suitable 5′ handle for any given species of interest (see, e.g, Tamulaitis et al. 2014, Mol Cell. 2014 Nov. 20; 56(4):506-17; Jia et al. 2019, Mol Cell. 2019 Jan. 17; 73(2): 264-277.e5; Bouillon et al., Mol Cell. 2013 Oct. 10; 52(1): 124-134; and Staak et al., Mol Cell. 2013 Oct. 10; 52(1): 135-145).

A guide RNA and a subject multi-subunit Type III CRISPR-Cas effector polypeptide form a complex (e.g., bind via non-covalent interactions). The guide RNA provides target specificity to the complex via the guide sequence (targeting sequence). In other words, the multi-subunit Type III CRISPR-Cas effector polypeptide is guided to a target nucleic acid sequence (e.g. a target sequence) by virtue of its association with the guide RNA.

In some embodiments, guide RNA molecules may be extended to include sites for the binding of RNA binding proteins. In some embodiments, multiple guide RNAs can be assembled into a pre-crRNA array, which allows for multiplex editing to enable simultaneous targeting to several sites.

A guide RNA (gRNA) may be expressed in a variety of ways as will be apparent to one of skill in the art. For example, a gRNA may be expressed from a recombinant nucleic acid in vivo, from a recombinant nucleic acid in vitro, from a recombinant nucleic acid ex vivo, or can be synthetically synthesized. In some cases, expression of a guide RNA is driven by a Pol III promoter (e.g., U6, H1, and the like).

A guide RNA of the present disclosure may have various nucleotide lengths. A guide RNA may contain, for example, at least 20, at least 25, at least 30, at least 35, at least 40, at least at least 60, at least 70, at least 80, at least 90, at least 100, at least 110, at least 120, at least 130, at least 140, at least 150, at least 160, at least 170, at least 180 nucleotides, at least 190 nucleotides, or at least 200 nucleotides or more.

A guide RNA of the present disclosure may hybridize with a particular nucleotide sequence on a target nucleic acid. This hybridization may be 100% complementary or it may be less than 100% complementary so long as the hybridization is sufficient to allow a subject multi-subunit Type III CRISPR-Cas effector polypeptide to bind to or interact with the target nucleic acid. A guide RNA may contain a nucleotide sequence that is, for example, at least 80%, at least 85%, at least 90%, at least 91%, at least 92%, at least 93%, at least 94%, at least 95%, at least 96%, at least 97%, at least 98%, at least 99%, or 100% identical or complementary to the target nucleotide sequence in the target nucleic acid that is targeted by/to be hybridized with the guide RNA.

### Methods of Detecting a Target RNA

The present disclosure provides a method of detecting a target RNA in a eukaryotic cell. The method comprises contacting the target RNA in the cell with a complex (e.g., introducing into the eukaryotic cell one or more nucleic acids encoding the complex), the complex comprising: a) a Type III CRISPR-Cas effector polypeptide, wherein the Type III CRISPR-Cas effector polypeptide comprises at least 5 subunits, wherein the Type II CRISPR-Cas effector polypeptide does not substantially cleave the target RNA; and b) a guide RNA that comprises: i) a targeting region that comprises a nucleotide sequence that is complementary to a target sequence in the target RNA; and ii) a protein-binding region that binds to the Type III CRISPR-Cas effector polypeptide. In some cases, the Type III CRISPR-Cas effector polypeptide comprises Csm1-Csm5 subunits. In some cases, the Type III CRISPR-Cas effector polypeptide comprises Cmr1-Cmr6 subunits.

In some cases, the Type III CRISPR-Cas effector polypeptide lacks RNase activity and instead only binds to the target RNA. In other words, the catalytic RNA cleavage activity (RNase activity) of the protein (e.g., Csm3) that naturally cleaves target RNA is inactivated by mutation. In yet other words, the protein (e.g., Csm3) is a variant having one or more mutations that ablate RNase activity. For example, in some cases, the RNA cleaving protein has a mutation at a position corresponding to D33 (e.g., D33A) of the Csm3 protein sequence of SEQ ID NO: 16. As such, in some cases, the multi-subunit Type III CRISPR-Cas effector polypeptide includes a Csm3 protein having a mutation at position D33 (e.g., D33A).

In some cases, one or more of the subunits comprises a label moiety. In some cases, the label moiety comprises a fluorescent moiety. In some cases, one or more of the subunits is a fusion protein comprising: i) the subunit; and ii) a fluorescent protein.

The terms “label”, “detectable label”, or “label moiety” as used herein refer to any moiety that provides for signal detection and may vary widely depending on the particular nature of the assay. Label moieties of interest include both directly detectable labels (direct labels; e.g., a fluorescent label) and indirectly detectable labels (indirect labels; e.g., a binding pair member). A fluorescent label can be any fluorescent label (e.g., a fluorescent dye (e.g., fluorescein, Texas red, rhodamine, ALEXAFLUOR® labels, and the like), a fluorescent protein (e.g., green fluorescent protein (GFP), enhanced GFP (EGFP), yellow fluorescent protein (YFP), red fluorescent protein (RFP), cyan fluorescent protein (CFP), cherry, tomato, tangerine, and any fluorescent derivative thereof), etc.). Suitable detectable (directly or indirectly) label moieties for use in the methods include any moiety that is detectable by spectroscopic, photochemical, biochemical, immunochemical, electrical, optical, chemical, or other means. For example, suitable indirect labels include biotin (a binding pair member), which can be bound by streptavidin (which can itself be directly or indirectly labeled). Labels can also include: a radiolabel (a direct label) (e.g., ³H, ¹²⁵I, ³⁵S, ¹⁴C, or ³²P); an enzyme (an indirect label) (e.g., peroxidase, alkaline phosphatase, galactosidase, luciferase, glucose oxidase, and the like); a fluorescent protein (a direct label)(e.g., green fluorescent protein, red fluorescent protein, yellow fluorescent protein, and any convenient derivatives thereof); a metal label (a direct label); a colorimetric label; a binding pair member; and the like. By “partner of a binding pair” or “binding pair member” is meant one of a first and a second moiety, wherein the first and the second moiety have a specific binding affinity for each other. Suitable binding pairs include, but are not limited to: antigen/antibodies (for example, digoxigenin/anti-digoxigenin, dinitrophenyl (DNP)/anti-DNP, dansyl-X-anti-dansyl, fluorescein/anti-fluorescein, lucifer yellow/anti-lucifer yellow, and rhodamine anti-rhodamine), biotin/avidin (or biotin/streptavidin) and calmodulin binding protein (CBP)/calmodulin. Any binding pair member can be suitable for use as an indirectly detectable label moiety.

In some cases, the target RNA to be detected is present in the nucleus. In some cases, the target RNA is present in the cytoplasm. In some cases, the target RNA is a coding RNA. In some cases, the coding RNA is an mRNA or a pre-mRNA. In some cases, the target RNA is a non-coding RNA. In some cases, the target RNA is a regulatory RNA. In some cases, the non-coding RNA is a transfer RNA (tRNA), a pre-ribosomal RNA, a ribosomal RNA (rRNA), a microRNA (miRNA), an enhancer RNA (eRNA), a Piwi-interacting RNA (piRNA), a small nucleolar RNA (snoRNA), a small nuclear RNA (snRNA), a small interfering RNA (siRNA), a circRNA, or a long non-coding RNA (lncRNA). In some cases, the target RNA is an endogenous RNA. In some cases, the target RNA is mitochondrial RNA or chloroplast RNA. In some cases, the target RNA is an exogenous RNA. In some cases, the exogenous RNA is a viral RNA.

### Recombinant Expression Vectors and Compositions

The present disclosure provides recombinant expression vectors, which can be used for carrying out a method of the present disclosure. A recombinant expression vector of the present disclosure comprises one or more nucleotide sequences encoding a multisubunit Type III CRISPR-Cas effector polypeptide comprising at least 5 subunits (e.g., comprising 5 subunits or comprising 6 subunits). A recombinant expression vector of the present disclosure can further include a nucleotide sequence encoding a Type III CRISPR-Cas guide RNA.

Examples of suitable recombinant expression vectors include a recombinant adeno-associated virus vector, a recombinant lentivirus vector, a recombinant adenovirus vector, and a recombinant retroviral vector.

In some cases, the nucleotide sequences encoding the at least 5 subunits are operably linked to a single promoter. In some cases, the nucleotide sequences encoding the at least 5 subunits are operably linked to two or more different promoters. Thus, e.g., in some cases, nucleotide sequences encoding the at least 5 subunits are each operably linked to a different promoter. In some cases, a first promoter is operably linked to nucleotide sequences encoding 2 of the at least 5 subunits; and a second promoter is operably linked to nucleotide sequences encoding the other 3 of the at least 5 subunits.

In some cases, the promoter is a constitutively active promoter. In some cases, the promoter is a regulatable promoter. In some cases, the promoter is an inducible promoter. In some cases, the promoter is a tissue-specific promoter. In some cases, the promoter is a cell type-specific promoter. In some cases, the transcriptional control element (e.g., the promoter) is functional in a targeted cell type or targeted cell population.

The present disclosure provides a composition useful for modifying a target RNA in a eukaryotic cell, the composition comprising: a) one or more nucleic acids comprising nucleotide sequences encoding a multi-subunit Type III CRISPR-Cas effector polypeptide, wherein the multi-subunit Type III CRISPR-Cas effector polypeptide comprises at least 5 subunits; and b) one or more guide RNAs, wherein each of the one or more guide RNAs comprises: i) a targeting region that comprises a nucleotide sequence that is complementary to a target sequence in the target RNA; and ii) a protein-binding region that binds to the multi-subunit Type III CRISPR-Cas effector polypeptide; or a nucleic acid comprising a nucleotide sequence encoding the guide RNA, wherein, when the eukaryotic cell is contacted with the composition, the multi-subunit Type III CRISPR-Cas effector polypeptide is produced in the cell and forms a complex with the guide RNA, and wherein the complex binds to the target RNA and results in modification of the target RNA in the cell.

In some cases, the one or more nucleic acids comprises one or more recombinant expression vectors. In some cases, the one or more recombinant expression vectors are selected from a recombinant adeno-associated virus vector, a recombinant lentivirus vector, a recombinant adenovirus vector, and a recombinant retroviral vector. In some cases, the nucleotide sequences encoding the at least 5 subunits are operably linked to one, two or more promoters, and wherein the promoters are constitutive or regulatable promoters in any combination. In some cases, the one or more nucleic acids comprising nucleotide sequences encoding the multi-subunit Type III CRISPR-Cas effector polypeptide comprise a nucleotide sequence encoding the guide RNA.

In some cases, the target RNA is present in the nucleus or in an organelle or present in the cytoplasm. In some cases, the target RNA is a coding RNA. In some cases, the coding RNA is an mRNA or a pre-mRNA. In some cases, the target RNA is a non-coding RNA. In some cases, the target RNA is a regulatory RNA. In some cases, the non-coding RNA is a transfer RNA (tRNA), a pre-ribosomal RNA, a ribosomal RNA (rRNA), a microRNA (miRNA), an enhancer RNA (eRNA), a Piwi-interacting RNA (piRNA), a small nucleolar RNA (snoRNA), a small nuclear RNA (snRNA), a small interfering RNA (siRNA), a circRNA, or a long non-coding RNA (lncRNA). In some cases, the target RNA is an endogenous or an exogenous RNA. In some cases, the exogenous RNA is a viral RNA.

In some cases, the modifying comprises cleavage of the target RNA. In some cases, the modifying comprises methylation or adenylation.

In some cases, the eukaryotic cell is a mammalian cell, a plant cell, an insect cell, a reptile cell, an amphibian cell, a protozoan cell, an arachnid cell, an avian cell, or a fish cell. In some cases, the eukaryotic cell is in vitro or in vivo.

In some cases, the multi-subunit Type III CRISPR-Cas effector polypeptide is a Type IIIA CRISPR-Cas effector polypeptide comprising Cas10/Csm1, Csm2, Csm3, Csm4, and Csm5 polypeptides. In some cases, the Cas10/Csm1, Csm2, Csm3, Csm4, and Csm5 polypeptides each independently comprise an amino acid sequence having at least 50% amino acid sequence identity to any of the amino acid sequences of the Cas10/Csm1, Csm2, Csm3, Csm4, and Csm5 polypeptides depicted in FIG. FIG. 5A-5E (SEQ ID Nos: 1, 7, 16, 26, and 33, respectively) or FIG. FIG. 7A-7E (SEQ ID Nos: 2-6 (Csm1), 8-15 (Csm2), 17-25 (Csm3), 27-32 (Csm4), and 34-39 (Csm5)).

In some cases, the multi-subunit Type III CRISPR-Cas effector polypeptide is a Type IIIB CRISPR-Cas effector polypeptide comprising Cmr1, Cmr2, Cmr3, Cmr4, Cmr5, and Cmr6 subunits. In some cases, the Cmr1, Cmr2, Cmr3, Cmr4, Cmr5, and Cmr6 polypeptides each independently comprise an amino acid sequence having at least 50% amino acid sequence identity to any of the amino acid sequences of the Cmr1, Cmr2, Cmr3, Cmr4, Cmr5, and Cmr6 polypeptides depicted in FIG. 6A-6E (SEQ ID Nos: 40-45 (Cmr1), 46-50 (Cmr2), 51-55 (Cmr3), 56-61 (Cmr4), 62-66 (Cmr5), and 67-70 and 179-180 (Cmr6)).

In some cases, the multi-subunit Type III CRISPR-Cas effector polypeptide comprises one or more amino acid substitutions that reduce DNAse activity. In some cases, the one or more amino acid substitutions that reduce DNAse activity comprise a substitution of H15 (e.g., H15A), a substitution of D16 (e.g., D16A), or both H15 and D16 (e.g., H15A/D16A), of a Csm1 polypeptide. In some cases, the multi-subunit Type III CRISPR-Cas effector polypeptide comprises one or more amino acid substitutions that reduce polymerization of ATP into a cyclic oligoadenylate (cA) molecule. In some cases, the one or more amino acid substitutions that reduce polymerization of ATP to cA comprise a substitution of D577 (e.g., D577A), a substitution of D578 (e.g., D578A), or a substitution of both D577 and D578 (e.g., D577A/D578A) of a Csm10/Csm1 polypeptide.

In some cases, the multi-subunit Type III CRISPR-Cas effector polypeptide comprises one or more amino acid substitutions that reduce RNase activity. In some cases, the one or more amino acid substitutions that reduce RNase activity include a D33 (e.g., D33A) substitution of a Csm3 polypeptide. Such a polypeptide lacks RNase activity and instead only binds to the target RNA. For example, in some cases, the RNA cleaving protein has a mutation at a position corresponding to D33 (e.g., D33A) of the Csm3 protein sequence of SEQ ID NO: 16. As such, in some cases, the multi-subunit Type III CRISPR-Cas effector polypeptide includes a Csm3 protein having a mutation at position D33 (e.g., D33A).

As such, in some cases the Cas10/Csm1 polypeptide comprises an amino acid sequence having at least 50% (e.g., at least 60%, 70%, 75%, 80%, 85%, 90%, 95%, 97%, 98%, 99%, or 99.5%, or 100%) amino acid sequence identity to the amino acid sequence of any one of SEQ ID Nos: 1-6 (and in some such cases the Csm1 polypeptide is a variant with reduced DNAse activity and/or reduced reduce ATP polymerization activity (into cA) as discussed above). In some cases, the Cas10/Csm1 polypeptide comprises an amino acid sequence having at least 80% (e.g., at least 85%, 90%, 95%, 97%, 98%, 99%, or 99.5%, or 100%) amino acid sequence identity to the amino acid sequence of any one of SEQ ID Nos: 1-6 (and in some such cases the Csm1 polypeptide is a variant with reduced DNAse activity and/or reduced reduce ATP polymerization activity (into cA) as discussed above). In some cases, the Cas10/Csm1 polypeptide comprises an amino acid sequence having the amino acid sequence of any one of SEQ ID Nos: 1-6 (and in some such cases the Csm1 polypeptide is a variant with reduced DNAse activity and/or reduced reduce ATP polymerization activity (into cA) as discussed above).

In some cases the Csm2 polypeptide comprises an amino acid sequence having at least 50% (e.g., at least 60%, 70%, 75%, 80%, 85%, 90%, 95%, 97%, 98%, 99%, or 99.5%, or 100%) amino acid sequence identity to the amino acid sequence of any one of SEQ ID Nos: 7-15. In some cases, the Csm2 polypeptide comprises an amino acid sequence having at least 80% (e.g., at least 85%, 90%, 95%, 97%, 98%, 99%, or 99.5%, or 100%) amino acid sequence identity to the amino acid sequence of any one of SEQ ID Nos: 7-15. In some cases, the Csm2 polypeptide comprises an amino acid sequence having the amino acid sequence of any one of SEQ ID Nos: 7-15.

In some cases the Csm3 polypeptide comprises an amino acid sequence having at least 50% (e.g., at least 60%, 70%, 75%, 80%, 85%, 90%, 95%, 97%, 98%, 99%, or 99.5%, or 100%) amino acid sequence identity to the amino acid sequence of any one of SEQ ID Nos: 16-25 (and in some such cases the Csm3 polypeptide is a variant with ablated RNase activity, e.g., includes a mutation at a position corresponding to D33 (e.g., D33A) of SEQ ID NO: 16 as discussed above). In some cases, the Csm3 polypeptide comprises an amino acid sequence having at least 80% (e.g., at least 85%, 90%, 95%, 97%, 98%, 99%, or 99.5%, or 100%) amino acid sequence identity to the amino acid sequence of any one of SEQ ID Nos: 16-25 (and in some such cases the Csm3 polypeptide is a variant with ablated RNase activity, e.g., includes a mutation at a position corresponding to D33 (e.g., D33A) of SEQ ID NO: 16 as discussed above). In some cases, the Csm3 polypeptide comprises an amino acid sequence having the amino acid sequence of any one of SEQ ID Nos: 16-25 (and in some such cases the Csm3 polypeptide is a variant with ablated RNase activity, e.g., includes a mutation at a position corresponding to D33 (e.g., D33A) of SEQ ID NO: 16 as discussed above).

In some cases the Csm4 polypeptide comprises an amino acid sequence having at least 50% (e.g., at least 60%, 70%, 75%, 80%, 85%, 90%, 95%, 97%, 98%, 99%, or 99.5%, or 100%) amino acid sequence identity to the amino acid sequence of any one of SEQ ID Nos: 26-32. In some cases, the Csm4 polypeptide comprises an amino acid sequence having at least 80% (e.g., at least 85%, 90%, 95%, 97%, 98%, 99%, or 99.5%, or 100%) amino acid sequence identity to the amino acid sequence of any one of SEQ ID Nos: 26-32. In some cases, the Csm4 polypeptide comprises an amino acid sequence having the amino acid sequence of any one of SEQ ID Nos: 26-32.

In some cases the Csm5 polypeptide comprises an amino acid sequence having at least 50% (e.g., at least 60%, 70%, 75%, 80%, 85%, 90%, 95%, 97%, 98%, 99%, or 99.5%, or 100%) amino acid sequence identity to the amino acid sequence of any one of SEQ ID Nos: 33-39. In some cases, the Csm5 polypeptide comprises an amino acid sequence having at least 80% (e.g., at least 85%, 90%, 95%, 97%, 98%, 99%, or 99.5%, or 100%) amino acid sequence identity to the amino acid sequence of any one of SEQ ID Nos: 33-39. In some cases, the Csm5 polypeptide comprises an amino acid sequence having the amino acid sequence of any one of SEQ ID Nos: 33-39.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
Aspect 1. A method for modifying a target RNA in a eukaryotic cell, the method comprising introducing into the eukaryotic cell:
a) one or more nucleic acids comprising nucleotide sequences encoding a multi-subunit Type III CRISPR-Cas effector polypeptide, wherein the multi-subunit Type III CRISPR-Cas effector polypeptide comprises at least 5 subunits; and
b) one or more guide RNAs, wherein each of the one or more guide RNAs comprises: i) a targeting region that comprises a nucleotide sequence that is complementary to a target sequence in the target RNA; and ii) a protein-binding region that binds to the multi-subunit Type III CRISPR-Cas effector polypeptide; or a nucleic acid comprising a nucleotide sequence encoding the guide RNA,
wherein the multi-subunit Type III CRISPR-Cas effector polypeptide is produced in the cell and forms a complex with the guide RNA, and wherein the complex binds to the target RNA and results in modification of the target RNA in the cell.
Aspect 2. The method of aspect 1, wherein the one or more nucleic acids comprises one or more recombinant expression vectors.
Aspect 3. The method of aspect 2, wherein one or more recombinant expression vectors are selected from a recombinant adeno-associated virus vector, a recombinant lentivirus vector, a recombinant adenovirus vector, and a recombinant retroviral vector.
Aspect 4. The method of any one of aspects 1-3, wherein the nucleotide sequences encoding the at least 5 subunits are operably linked to a single promoter.
Aspect 5. The method of any one of aspects 1-3, wherein the nucleotide sequences encoding the at least 5 subunits are operably linked to two or more different promoters.
Aspect 6. The method of any one of aspects 1-5, wherein the promoter is a constitutive promoter.
Aspect 7. The method of any one of aspects 1-5, wherein the promoter is a regulatable promoter.
Aspect 8. The method of any one of aspects 1-7, wherein the one or more nucleic acids comprising nucleotide sequences encoding the multi-subunit Type III CRISPR-Cas effector polypeptide comprise a nucleotide sequence encoding the guide RNA.
Aspect 9. The method of any one of aspects 1-8, wherein the target RNA is present in the nucleus or in an organelle.
Aspect 10. The method of any one of aspects 1-8, wherein the target RNA is present in the cytoplasm.
Aspect 11. The method of any one of aspects 1-10, wherein the target RNA is a coding RNA.
Aspect 12. The method of aspect 11, wherein the coding RNA is an mRNA or a pre-mRNA.
Aspect 13. The method of any one of aspects 1-10, wherein the target RNA is a non-coding RNA.
Aspect 14. The method of aspect 13, wherein the target RNA is a regulatory RNA.
Aspect 15. The method of aspect 13, wherein the non-coding RNA is a transfer RNA (tRNA), a pre-ribosomal RNA, a ribosomal RNA (rRNA), a microRNA (miRNA), an enhancer RNA (eRNA), a Piwi-interacting RNA (piRNA), a small nucleolar RNA (snoRNA), a small nuclear RNA (snRNA), a small interfering RNA (siRNA), a circRNA, or a long non-coding RNA (lncRNA).
Aspect 16. The method of any one of aspects 1-15, wherein the target RNA is an endogenous RNA.
Aspect 17. The method of any one of aspects 1-15, wherein the target RNA is mitochondrial RNA or chloroplast RNA.
Aspect 18. The method of any one of aspects 1-15, wherein the target RNA is an exogenous RNA.
Aspect 19. The method of aspect 18, wherein the exogenous RNA is a viral RNA.
Aspect 20. The method of any one of aspects 1-19, wherein the modifying comprises cleavage of the target RNA.
Aspect 21. The method of any one of aspects 1-19, wherein the modifying comprises methylation or adenylation.
Aspect 22. The method of any one of aspects 1-21, wherein the eukaryotic cell is a mammalian cell, a plant cell, an insect cell, a reptile cell, an amphibian cell, a protozoan cell, an arachnid cell, an avian cell, or a fish cell.
Aspect 23. The method of any one of aspects 1-22, wherein the eukaryotic cell is in vitro.
Aspect 24. The method of any one of aspects 1-22, wherein the eukaryotic cell is in vivo.
Aspect 25. The method of any one of aspects 1-24, wherein the multi-subunit Type III CRISPR-Cas effector polypeptide is a Type IIIA CRISPR-Cas effector polypeptide comprising Cas10/Csm1, Csm2, Csm3, Csm4, and Csm5 polypeptides.
Aspect 26. The method of aspect 25, wherein the Cas10/Csm1, Csm2, Csm3, Csm4, and Csm5 polypeptides each independently comprise an amino acid sequence having at least 50% amino acid sequence identity to any of the amino acid sequences of the Cas10/Csm1, Csm2, Csm3, Csm4, and Csm5 polypeptides depicted in FIG. 5A-5E or FIG. 7A-7E.
Aspect 27. The method of any one of aspects 1-24, wherein the multi-subunit Type III CRISPR-Cas effector polypeptide is a Type IIIB CRISPR-Cas effector polypeptide comprising Cmr1, Cmr2, Cmr3, Cmr4, Cmr5, and Cmr6 subunits.
Aspect 28. The method of aspect 27, wherein the Cmr1, Cmr2, Cmr3, Cmr4, Cmr5, and Cmr6 polypeptides each independently comprise an amino acid sequence having at least 50% amino acid sequence identity to any of the amino acid sequences of the Cmr1, Cmr2, Cmr3, Cmr4, Cmr5, and Cmr6 polypeptides depicted in FIG. 6A-6F.
Aspect 29. The method of any one of aspects 1-28, wherein the multi-subunit Type III CRISPR-Cas effector polypeptide comprises one or more amino acid substitutions that reduce DNAse activity.
Aspect 30. The method of aspect 29, wherein the one or more amino acid substitutions that reduce DNAse activity comprise a substitution of H15, a substitution of D16, or both H15 and D16, of a Csm1 polypeptide.
Aspect 31. The method of any one of aspects 1-30, wherein the multi-subunit Type III CRISPR-Cas effector polypeptide comprises one or more amino acid substitutions that reduce polymerization of ATP into a cyclic oligoadenylate (cA) molecule.
Aspect 32. The method of aspect 31, wherein the one or more amino acid substitutions that reduce polymerization of ATP to cA comprise a substitution of D577, a substitution of D578, or a substitution of both D577 and D578 of a Csm10/Csm1 polypeptide.
Aspect 33. A method of detecting a target RNA in a eukaryotic cell, the method comprising contacting the target RNA with a complex comprising:
a) a Type III CRISPR-Cas effector polypeptide, wherein the Type III CRISPR-Cas effector polypeptide comprises 5 subunits, wherein the Type II CRISPR-Cas effector polypeptide does not substantially cleave the target RNA; and
b) a guide RNA that comprises: i) a targeting region that comprises a nucleotide sequence that is complementary to a target sequence in the target RNA; and ii) a protein-binding region that binds to the Type III CRISPR-Cas effector polypeptide.
Aspect 34. The method of aspect 33, wherein one or more of the subunits comprises a detectable label.
Aspect 35. The method of aspect 34, wherein the detectable label comprises a fluorescent moiety.
Aspect 36. The method of any one of aspects 33-35, wherein the target RNA is present in the nucleus.
Aspect 37. The method of any one of aspects 33-35, wherein the target RNA is present in the cytoplasm.
Aspect 38. The method of any one of aspects 33-37, wherein the target RNA is a coding RNA.
Aspect 39. The method of aspect 38, wherein the coding RNA is an mRNA or a pre-mRNA.
Aspect 40. The method of any one of aspects 33-37, wherein the target RNA is a non-coding RNA.
Aspect 41. The method of aspect 40, wherein the target RNA is a regulatory RNA.
Aspect 42. The method of aspect 40, wherein the non-coding RNA is a transfer RNA (tRNA), a pre-ribosomal RNA, a ribosomal RNA (rRNA), a microRNA (miRNA), an enhancer RNA (eRNA), a Piwi-interacting RNA (piRNA), a small nucleolar RNA (snoRNA), a small nuclear RNA (snRNA), a small interfering RNA (siRNA), a circRNA, or a long non-coding RNA (lncRNA).
Aspect 43. The method of any one of aspects 33-42, wherein the target RNA is an endogenous RNA.
Aspect 44. The method of any one of aspects 33-42, wherein the target RNA is mitochondrial RNA or chloroplast RNA.
Aspect 45. The method of any one of aspects 33-42, wherein the target RNA is an exogenous RNA.
Aspect 46. The method of aspect 45, wherein the exogenous RNA is a viral RNA.
Aspect 47. A composition useful for modifying a target RNA in a eukaryotic cell, the composition comprising:
a) one or more nucleic acids comprising nucleotide sequences encoding a multi-subunit Type III CRISPR-Cas effector polypeptide, wherein the multi-subunit Type III CRISPR-Cas effector polypeptide comprises at least 5 subunits; and
b) one or more guide RNAs, wherein each of the one or more guide RNAs comprises: i) a targeting region that comprises a nucleotide sequence that is complementary to a target sequence in the target RNA; and ii) a protein-binding region that binds to the multi-subunit Type III CRISPR-Cas effector polypeptide; or a nucleic acid comprising a nucleotide sequence encoding the guide RNA,
wherein, when the eukaryotic cell is contacted with the composition, the multi-subunit Type III CRISPR-Cas effector polypeptide is produced in the cell and forms a complex with the guide RNA, and wherein the complex binds to the target RNA and results in modification of the target RNA in the cell.
Aspect 48. The composition of aspect 47, wherein the one or more nucleic acids comprises one or more recombinant expression vectors.
Aspect 49. The composition of aspect 48, wherein one or more recombinant expression vectors are selected from a recombinant adeno-associated virus vector, a recombinant lentivirus vector, a recombinant adenovirus vector, and a recombinant retroviral vector.
Aspect 50. The composition of any one of aspects 47-49, wherein the nucleotide sequences encoding the at least 5 subunits are operably linked to one, two or more promoters, and wherein the promoters are constitutive or regulatable promoters in any combination.
Aspect 51. The composition of any one of aspects 47-50, wherein the one or more nucleic acids comprising nucleotide sequences encoding the multi-subunit Type III CRISPR-Cas effector polypeptide comprise a nucleotide sequence encoding the guide RNA.
Aspect 52. The composition of any one of aspects 47-51, wherein the target RNA is present in the nucleus or in an organelle or present in the cytoplasm.
Aspect 53. The composition of any one of aspects 47-52, wherein the target RNA is a coding RNA.
Aspect 54. The composition of aspect 53, wherein the coding RNA is an mRNA or a pre-mRNA.
Aspect 55. The composition of any one of aspects 47-54, wherein the target RNA is a non-coding RNA.
Aspect 56. The composition of aspect 55, wherein the target RNA is a regulatory RNA.
Aspect 57. The composition of aspect 56, wherein the non-coding RNA is a transfer RNA (tRNA), a pre-ribosomal RNA, a ribosomal RNA (rRNA), a microRNA (miRNA), an enhancer RNA (eRNA), a Piwi-interacting RNA (piRNA), a small nucleolar RNA (snoRNA), a small nuclear RNA (snRNA), a small interfering RNA (siRNA), a circRNA, or a long non-coding RNA (lncRNA).
Aspect 58. The composition of any one of aspects 47-57, wherein the target RNA is an endogenous or an exogenous RNA.
Aspect 59. The composition of aspect 58, wherein the exogenous RNA is a viral RNA.
Aspect 60. The composition of any one of aspects 47-59, wherein the modifying comprises cleavage of the target RNA.
Aspect 61. The composition of any one of aspects 47-60, wherein the modifying comprises methylation or adenylation.
Aspect 62. The composition of any one of aspects 47-61, wherein the eukaryotic cell is a mammalian cell, a plant cell, an insect cell, a reptile cell, an amphibian cell, a protozoan cell, an arachnid cell, an avian cell, or a fish cell.
Aspect 63. The composition of any one of aspects 47-62, wherein the eukaryotic cell is in vitro or in vivo.
Aspect 64. The composition of any one of aspects 47-63, wherein the multi-subunit Type III CRISPR-Cas effector polypeptide is a Type IIIA CRISPR-Cas effector polypeptide comprising Cas10/Csm1, Csm2, Csm3, Csm4, and Csm5 polypeptides.
Aspect 65. The composition of aspect 64, wherein the Cas10/Csm1, Csm2, Csm3, Csm4, and Csm5 polypeptides each independently comprise an amino acid sequence having at least 50% amino acid sequence identity to the amino acid sequences of the Cas10/Csm1, Csm2, Csm3, Csm4, and Csm5 polypeptides depicted in FIG. 5.
Aspect 66. The composition of any one of aspects 47-65, wherein the multi-subunit Type III CRISPR-Cas effector polypeptide is a Type IIIB CRISPR-Cas effector polypeptide comprising Cmr1, Cmr2, Cmr3, Cmr4, Cmr5, and Cmr6 subunits.
Aspect 67. The composition of aspect 66, wherein the Cmr1, Cmr2, Cmr3, Cmr4, Cmr5, and Cmr6 polypeptides each independently comprise an amino acid sequence having at least 50% amino acid sequence identity to the amino acid sequences of the Cmr1, Cmr2, Cmr3, Cmr4, Cmr5, and Cmr6 polypeptides depicted in FIG. 6.
Aspect 68. The composition of any one of aspects 47-67, wherein the multi-subunit Type III CRISPR-Cas effector polypeptide comprises one or more amino acid substitutions that reduce DNAse activity.
Aspect 69. The composition of aspect 68, wherein the one or more amino acid substitutions that reduce DNAse activity comprise a substitution of H15, a substitution of D16, or both H15 and D16, of a Csm1 polypeptide.
Aspect 70. The composition of any one of aspects 47-69, wherein the multi-subunit Type III CRISPR-Cas effector polypeptide comprises one or more amino acid substitutions that reduce polymerization of ATP into a cyclic oligoadenylate (cA) molecule.
Aspect 71. The composition of aspect 70, wherein the one or more amino acid substitutions that reduce polymerization of ATP to cA comprise a substitution of D577, a substitution of D578, or a substitution of both D577 and D578 of a Csm10/Csm1 polypeptide.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Methods (for Example 1 and Example 2)

### Cell Lines and Culture Conditions

HEKf293T, HEK293T-GFP, and HEK293T-GFP/RFP cells (UC Berkeley Cell Culture Facility) were grown in medium containing DMEM, high glucose, GlutaMAX supplement, sodium pyruvate (Thermo Fisher Scientific), 10% FBS (Sigma), 25 mM HEPES pH 7.2-7.5 (Thermo Fisher Scientific), 1×MEM non-essential amino acids (Thermo Fisher Scientific), 1× Pen/Strep (Thermo Fisher Scientific), and 0.1 mM BME (Thermo Fisher Scientific) at 37 C with 5% CO2. All cell lines were verified to be Mycoplasma-free (abm, PCR Mycoplasma detection kit).

### Plasmid Construction and Cloning

Csm CRISPR-Cas sequences were derived from Streptococcus thermophilus strain ND03. Protein sequences were human codon-optimized using online tools (GenScript), synthesized as gene blocks (IDT), modified using PCR, and cloned into custom eukaryotic expression vectors (derived from pUC19) by Golden Gate assembly, Gibson assembly (NEB), or Gibson assembly Ultra (Synthetic Genomics). Plasmids were verified by Sanger or whole-plasmid sequencing. All cloning was performed in Stb13 E. coli (Thermo Fisher Scientific) to prevent recombination between repetitive sequences. Sequences are provided in the tables below.

### DNA Transfections

1×10\hat{ }6 HEK293T cells were transfected with 5 ug plasmid DNA using 15 ul FuGENE HD transfection reagent in 6-well plates as per manufacturer's instructions. Cells were grown for 48 hr post-transfection to allow protein expression and RNA KD to occur, unless otherwise stated.

### Flow Cytometry

Cell fluorescence was assayed on an Attune NxT acoustic focusing cytometer (Thermo Fisher Scientific) equipped with 488 nm excitation laser and 530/30 emission filter (GFP), and 561 nm excitation laser and 620/15 emission filter (mCherry). Data were analyzed using Attune Cytometric Software v5.1.1 and FlowJo v10.7.1.

### Fluorescence Activated Cell Sorting (FACS)

Cells were sorted by fluorescence on a Sony Cell Sorter SH800Z (100 um sorting chip) equipped with 488 nm excitation laser and 525/50 emission filter (GFP), and 561 nm excitation laser and 600/60 emission filter (mCherry). Data were analyzed using Sony Cell Sorter Software v2.1.5.

### Reverse Transcription-Quantitative Polymerase Chain Reaction (RT-qPCR)

Total cell RNA was extracted using TRIzol Reagent (Thermo Fisher Scientific) as per manufacturer's instructions. Genomic DNA was removed using TURBO DNase (Thermo Fisher Scientific). After inactivating TURBO DNase with DNase Inactivating Reagent, 1 ug DNase-free RNA was reverse transcribed using SuperScript III Reverse Transcriptase (Thermo Fisher Scientific) with random primers (Promega) as per manufacturer's instructions. qPCR was performed using iTaq Universal SYBR Green Supermix (Bio-Rad) in a CFX96 Real-Time PCR Detection System (Bio-Rad). Sequences are provided in the tables below.

### Cell Viability and Proliferation Assay

The WST-1 assay was used to quantify cell viability and proliferation. Cells transfected with Csm, Cas13, or shRNA constructs were grown in 96-well plates until the indicated timepoints, incubated with WST-1 reagent (Sigma) at 37 C for 1 hr as per manufacturer's instructions, and absorbance measured using a Cytation 5 microplate reader (BioTek Instruments) at 450 nm with 600 nm reference.

### Microscopy

For wide-field fluorescent imaging, cells were observed on a Zeiss Axio Observer Z1 inverted fluorescence microscope, equipped with 63×/1.4 NA oil DIC and 100×/1.4 NA oil Ph3 Plan Apochromat objective lenses, ORCA-Flash4.0 camera (Hamamatsu), and ZEN 2012 software. Images were generated using ZEN 2012 (Zeiss) and FIJI (ImageJ) software. For live-cell imaging, cells were grown on chambered #1.5 coverglasses (Nunc Lab-Tek II) in medium lacking phenol red (Thermo Fisher Scientific) and imaged directly on the inverted fluorescent microscope.

### RNA Fluorescence In Situ Hybridization (FISH)

Cells were grown on glass coverslips and rinsed in PBS. They were permeabilized in PBS/0.5% Triton X-100 for 10 min and then fixed in 4% paraformaldehyde for 10 min at room temp. Cells were dehydrated in a series of 70%, 80%, 90%, and 100% ethanol for 5 min each. Labeled oligo probe pool (10 nM final) was added to hybridization buffer containing 25% formamide, 2×SSC, 10% dextran sulfate, and nonspecific competitor (0.1 mg/mL human Cot-1 DNA [Thermo Fisher Scientific]). Hybridization was performed in a humidified chamber at 37 C overnight. After being washed 1× in 25% formamide/2×SSC at 37 C for 20 min and 3× in 2×SSC at 37 C for 5 min each, cells were mounted for wide-field fluorescent imaging. Nuclei were counter-stained with Hoechst 33342 (Life Technologies).

### FISH Probes

XIST oligo FISH probes were designed against the “Repeat D” region of human XIST RNA and synthesized by IDT carrying a 5′ Cy3 dye modification (see Table 4 for sequences). MALAT1 and NEAT1 oligo FISH probes were ordered from LGC Biosearch Technologies (SMF-2035-1, SMF-2036-1) carrying a Quasar 570 dye modification.

### Immunofluorescence

Cells were grown on glass coverslips and rinsed in phosphate buffered saline (PBS). They were fixed in 4% paraformaldehyde for 10 min and then permeabilized in PBS/0.5% Triton X-100 for 10 min at room temp. Cells were blocked with blocking buffer (PBS/0.05% Tween-20 containing 1% BSA) for 1 hr, incubated with primary antibody in blocking buffer for 1 hr, washed 3× with PBS/0.05% Tween-20 for 5 min each, incubated with dye-conjugated secondary antibody in blocking buffer for 1 hr at room temp, and washed 3× again with PBS/0.05% Tween-20 for 5 min each. Cells were mounted for wide-field fluorescent imaging and nuclei were counter-stained with Hoechst 33342 (Life Technologies).

### Western Blot

Cells were washed once with PBS and lysed in cold RIPA lysis buffer (50 mM Tris pH 7.5, 150 mM NaCl, 1% NP-40, 1% sodium deoxycholate, 0.1% SDS, 1× protease inhibitor cocktail [Sigma]). Lysate was sonicated (Qsonica Q800 Sonicator) in polystyrene tubes at 50% power setting, 30 sec on/30 sec off for a total sonication time of 5 min at 4 C. After removing debris by centrifugation at 16,000 g for 10 min, protein concentration in the supernatant was measured (Pierce BCA Assay Kit). 20-50 ug protein lysate was denatured in 1× Laemmli buffer at 95 C for 10 min and resolved by SDS-PAGE. Protein was transferred to Immun-Blot LF PVDF membrane (Bio-Rad). The membrane was blocked with blocking buffer (PBS/0.05% Tween-20 containing 5% milk) for 1 hr at room temp, incubated with primary antibody in blocking buffer overnight at 4 C, washed 3× with PBS/0.05% Tween-20 for 5 min each, incubated with dye-conjugated secondary antibody in blocking buffer for 1 hr at room temp, and washed 3× again with PBS/0.05% Tween-20 for 5 min each. Protein bands were visualized on a LI-COR Odyssey CLx with Image Studio v5.2 software using 700 nm and 800 nm channels.

### Antibodies

The following primary antibodies were used for Western blot: mouse anti-FLAG (Sigma, F1804), rabbit anti-GAPDH (Cell Signaling Technology, 14C10); for immunofluorescence: mouse anti-FLAG (Sigma, F1804). The following secondary antibodies were used for Western blot: IRDye 680RD goat anti-mouse (LI-COR, 926-68070), IRDye 800CW goat anti-rabbit (LI-COR, 926-32211); for immunofluorescence: Alexa Fluor 555 goat anti-mouse (Invitrogen, A21424).

### DNA-seq

Cells were lysed with Laird lysis buffer (10 mM Tris pH 8, 5 mM EDTA pH 8, 200 mM NaCl, 0.2% SDS, 0.2 mg/ml proteinase K) at 55° C. for 2 h and genomic DNA extracted with phenol-chloroform. The CKB locus was amplified from genomic DNA by PCR (primer sequences listed in the tables below) using PrimeSTAR GXL DNA polymerase (Takara Bio). The full-length PCR amplicon was purified from agarose gel and sheared by sonication to 200-400 bp fragments using a Qsonica Q800 Sonicator at 50% power setting, 30 s on/30 s off, for a total sonication time of 8 min at 4° C. DNA libraries were prepared using NEBNext Ultra II DNA Library Prep Kit for Illumina, as per manufacturer's instructions. Libraries were sequenced in-house (Center for Translational Genomics, UC Berkeley) on an iSeq100 with a 150 bp paired-end run configuration to a depth of ˜1 million reads each, with one biological replicate per sample.

### DNA-seq Analysis

Reads were aligned to the CKB (ENSG00000166165) gene locus with BWA MEM (v0.7.17) and PCR duplicates were removed with Picard Tools (v2.21.9). Mismatches and indels at each position were tabulated with Pysamstats (v1.1.2).

### RNA-seq

Total cell RNA was extracted using TRIzol Reagent (Thermo Fisher Scientific). Strand-specific cDNA libraries were prepared from polyA mRNA and sequenced using the Illumina NovaSeq paired-end 150 bp platform by Novogene. Libraries were sequenced to a depth of 30 million reads each, with 3 biological replicates per sample.

RNA-seq Analysis

Custom scripts were used for transcriptomic analysis. Briefly, reads were assessed for sequencing quality with FastQC, then adapters and low-quality bases were trimmed with CutAdapt. Samples were aligned to the GRCh38 reference genome (GENCODE Release 39) with STAR and uniquely mapped reads were used to generate a count matrix with FeatureCounts. EdgeR was used to normalize read counts and identify differentially expressed genes. Genes with a fold-change ≥2 relative to the untransfected sample were considered differentially expressed. Off-target editing was interrogated through sequence-similarity, by aligning crRNA sequences to the human transcriptome (GRCh38 cDNA, ENSEMBL release 105) with blastn and lenient parameters (E-value=10000, word_size=5, perc_identity 0.6). Potential off-targets were limited to BLAST results with 7 or fewer mismatched bases.

### Statistical Analysis

All graphs display the mean and standard deviation of 3 biological replicates. For RNA-seq analysis, no statistical parameters were applied given there was one biological replicate.

### Results

### Establishing an All-In-One Type III CRISPR-Cas System in Mammalian Cells

The Type III-A Csm complex from Streptococcus thermophilus was chosen for several reasons: 1. it has been extensively characterized biochemically, structurally, and in bacteria (Staals et al. 2014; Zhu et al. 2018; You et al. 2019; Tamulaitis et al. 2014; Jia et al. 2019; Guo et al. 2019; T. Y. Liu, Iavarone, and Doudna 2017; Mogila et al. 2019); 2. It functions optimally at 37 C; 3. It has been demonstrated to work in zebrafish upon ribonucleoprotein (RNP) microinjection (Fricke et al. 2020); and 4. It has fewer components than the analogous Type III-B Cmr complex (Staals et al. 2013). Proper expression of each individual protein component (Csm1-5 and Cas6) in immortalized human embryonic kidney (HEK293T) cells was verified. Proteins were human-codon-optimized, N-terminally FLAG-tagged for detection, and expressed from a CMV promoter. RNAi operates in the cytoplasm where mRNAs mainly reside. Each Csm component was localized to the nucleus through the addition of an N-terminal SV40 nuclear localization signal (NLS) so as to target nuclear RNAs as well as pre-mRNAs prior to export. Following transient transfection, Western blot (FIG. 1D) and immunofluorescence staining (FIG. 1E) verified proper size, expression, and nuclear localization of each protein.

To test the system, eGFP (henceforth “GFP”) mRNA was targeted in a GFP-expressing HEK293T cell line. Seven plasmids individually expressing Csm1-5, Cas6, and either a GFP-targeting or non-targeting crRNA from a U6 promoter were co-transfected into cells, and GFP fluorescence assayed by flow cytometry 48 hr post-transfection. Note that this strategy does not allow for any means to select cells into which all plasmids were successfully delivered, and will thus under-report knockdown (KD) efficiency. % GFP knockdown (KD) was calculated by dividing the mean fluorescence intensity (MFI) of cells transfected with the GFP-targeting crRNA by that of cells transfected with the non-targeting crRNA. ˜25% KD was observed using any of three crRNAs targeting different regions of the GFP ORF (FIG. 1F). Importantly, no KD was seen after transfecting the GFP-targeting crRNA and its processing factor (Cas6) alone (FIG. 1G), indicating that KD was not due to an antisense RNA effect. Furthermore, whereas ablating DNase (H15A, D16A) and cA synthesis (D577A, D578A) activities in Csm1 did not affect GFP KD, ablating RNase activity (D33A) in Csm3 completely abolished GFP KD (FIG. 1G), indicating that RNase activity is necessary and sufficient for KD.

Next, crRNA parameters were examined. Naturally occurring spacers for SthCsm crRNAs range from −30-45 nucleotides (nt) in length, although in vitro, spacers as short as 27 nt are sufficient to trigger all three catalytic activities (You et al. 2019). The GFP-targeting spacer length was varied from 24-48 nt in increments of 4 and assayed GFP KD. A length of 32 nt yielded the highest KD for the crRNA tested (FIG. 1H), with no KD seen for lengths ≤28 nt, and diminishing KD seen for lengths ≥32 nt. A more large-scale analysis must be performed to determine whether optimal spacer length differs from sequence to sequence. Next, the potential to multiplex crRNAs against multiple targets was examined. Two crRNAs were encoded within a single array—one targeting GFP and the other targeting mCherry (henceforth “RFP”)—and KD of GFP and RFP was examined in a HEK293T cell line expressing both (FIG. 1I). ˜25% KD was achieved for both GFP and RFP regardless of the order of crRNAs in the array (GFP-RFP or RFP-GFP), comparable to KD efficiency when targeting GFP or RFP alone. Together, these results demonstrate broad multiplexing capability for the Csm system.

Delivery of Csm by consolidating all components into a single vector was carried out. For this, two approaches were pursued concurrently: 1. expression of each protein from separate promoters, or 2. expression of all proteins from a single bidirectional promoter separated by 2A peptides (FIG. 1J). An RFP-encoding nucleotide sequence was included in the plasmid backbone to allow identification of transfected cells and thus more accurate measurement of KD efficiency. After re-confirming proper expression of all protein components by Western blot for both plasmids (FIG. 1K), it was found that both strategies (after optimizing the order of proteins in the single-promoter arrangement) led to ˜50% GFP KD in transfected cells (FIG. 1L). In summary, the single-promoter design is well-equipped for promoter-swapping and thus use in specific cell types or other eukaryotic systems, while the modular design of the separate-promoter vector allows for easy swapping or modification of individual Csm components.

FIG. 1A-1L. Establishing an all-in-one Type III CRISPR-Cas system in mammalian cells. A. Diagram showing cis- and trans-cleavage of Cas13. B. Diagram showing Type III-A CRISPR-Cas locus. The CRISPR array is transcribed and processed into mature crRNAs by Cas6, which assemble with Csm proteins. cA, cyclic oligoadenylate. C. Close-up of crRNA:target binding and cleavage, showing the 6-nt spacing pattern. D. Western blot showing proper size and expression of Csm proteins (red) in HEK293T cells. GAPDH shown as loading control (green). Arrows indicate faint bands. L, ladder; U, untransfected; 1-6, Csm1-5 and Cas6. E. Immunofluorescence showing expression and nuclear localization of Csm proteins in HEK293T cells. Labeling same as in (D). F. Relative GFP fluorescence (=MFI targeting crRNA/MFI non-targeting crRNA) of HEK293T-GFP cells transfected with the indicated crRNAs, measured by flow cytometry. Error bars indicate mean±standard deviation of 3 biological replicates. G. Relative GFP fluorescence of HEK293T-GFP cells transfected with the indicated protein complexes (or crRNA and Cas6 only), measured by flow cytometry. H. Relative GFP fluorescence of HEK293T-GFP cells transfected with crRNAs of indicated spacer length, measured by flow cytometry. I. Relative GFP and RFP fluorescence of HEK293T-GFP/RFP cells transfected with the indicated crRNAs (individual or multiplexed), measured by flow cytometry. J. Diagram showing all-in-one delivery vector designs. K. Western blot showing proper size and expression of Csm proteins (red) in HEK293T cells. GAPDH shown as loading control (green). Arrows indicate faint bands. Labeling same as in (D). L. Relative GFP fluorescence of HEK293T-GFP cells transfected with the indicated delivery vectors and crRNAs, measured by flow cytometry.

### Robust Knockdown of Endogenous Nuclear and Cytoplasmic RNAs

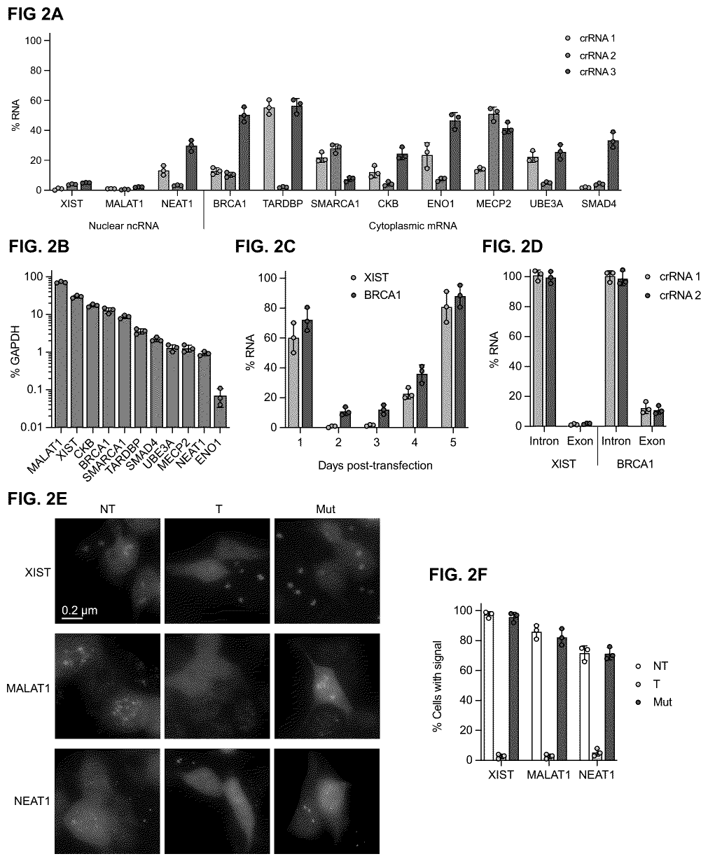

Thus far, Csm was used to KD highly overexpressed, heterologous GFP/RFP transgenes and assayed KD at the protein level (half-life >24 hours (Corish and Tyler-Smith 1999)), which may not accurately reflect abundance at the RNA level. It was sought to target endogenous transcripts and assay RNA KD directly. A panel of three nuclear noncoding RNAs (XIST, MALAT1, NEAT1) and eight cytoplasmic mRNAs (BRCA1, TARDBP, SMARCA1, CKB, ENO1, MECP2, UBE3A, SMAD4) (FIG. 2A) of varying abundances (FIG. 2B) was targeted; and three individual crRNAs was tested for each. HEK293T cells were transfected, transfected (RFP-positive) cells were isolated by FACS after 48 hr, total cell RNA extracted, and RNA KD assayed by RT-qPCR. A >90% KD was achieved for all eleven RNAs with at least one crRNA, compared to non-targeting crRNA control (FIG. 2A). These results demonstrate the Csm system to be a highly robust and efficient RNA KD tool for not only cytoplasmic but also nuclear RNAs, which are typically recalcitrant to KD by conventional RNAi methods (Behlke 2016).

To examine KD kinetics, the above RT-qPCR experiment was repeated for two of the RNA targets (XIST, BRCA1) across a 5-day time-course. KD peaked 2-3 days post-transfection and waned thereafter (FIG. 2C), as might be expected from the transient transfection method used to deliver the Csm into cells. The KD efficiency of crRNAs targeting intronic versus exonic regions was compared for the same two RNAs (FIG. 2D). Targeting introns did not lead to any noticeable reduction in mature RNA levels, possibly because their excision from pre-mRNA occurs more rapidly than their binding and cleavage by Csm.

To corroborate RNA KD with an orthogonal method, RNA FISH was performed for all three nuclear noncoding RNAs, which are easily visualized and display characteristic morphologies. HEK293T cells were transfected with Csm plasmid carrying a GFP reporter (to identify transfected cells) and either a targeting or non-targeting crRNA, and assayed by RNA FISH after 48 hr. XIST, MALAT1, and NEAT1 were all readily detected when delivering a non-targeting crRNA control (FIG. 2E,F). By contrast, use of a single targeting crRNA abolished all visible signal for each target RNA in transfected cells (GFP-positive cells), whereas signal was detected in untransfected (GFP-negative) cells. For further validation, delivery of targeting crRNA with RNase-inactivated Csm fully restored detection of each target RNA. Thus, near-complete target RNA KD with active Csm complexes was demonstrated using both molecular and microscopy-based techniques.

FIG. 2A-2F. Robust knockdown of endogenous nuclear and cytoplasmic RNAs. A. Relative RNA abundance (normalized to non-targeting crRNA) of the indicated targets in HEK293T cells transfected with the indicated crRNAs, measured by RT-qPCR. Error bars indicate mean±standard deviation of 3 biological replicates. B. Relative RNA abundance (normalized to GAPDH) of the indicated targets in untransfected HEK293T cells, measured by RT-qPCR. C. Relative RNA abundance (normalized to non-targeting crRNA) of XIST and BRCA1 in HEK293T cells at the indicated times post crRNA transfection, measured by RT-qPCR. D. Relative RNA abundance (normalized to non-targeting crRNA) of XIST and BRCA1 in HEK293T cells transfected with intron- or exon-targeting crRNAs, measured by RT-qPCR. E. RNA FISH (red) for the indicated targets in HEK293T cells transfected with targeting (T) or non-targeting (NT) crRNA, and RNase-active or -inactive (Mut) protein complex. Untransfected cells serve as internal control for transfected (green) cells. F. Quantification of (E). 100 transfected cells were counted for each condition.

RNA Knockdown with Minimal Off-Targets or Cytotoxicity

To examine off-target effects of Csm-mediated RNA KD and compare them to other established KD technologies, RNA-seq analysis was performed. XIST, MALAT1, CKB, or SMAD4 was knocked down for two days using Csm, Cas13 (RfxCas13d), or RNAi (shRNA) with crRNAs/shRNAs targeting the same region in each transcript (Wei et al. 2021; Bofill-De Ros and Gu 2016; Wessels et al. 2020). Differential expression analysis was performed by comparison to both untransfected and non-targeting crRNA/shRNA control samples. This allowed the assessment as to whether delivery of the Csm system itself causes any significant changes in RNA levels due to nonspecific cleavage by Cas6 or Csm3, etc.

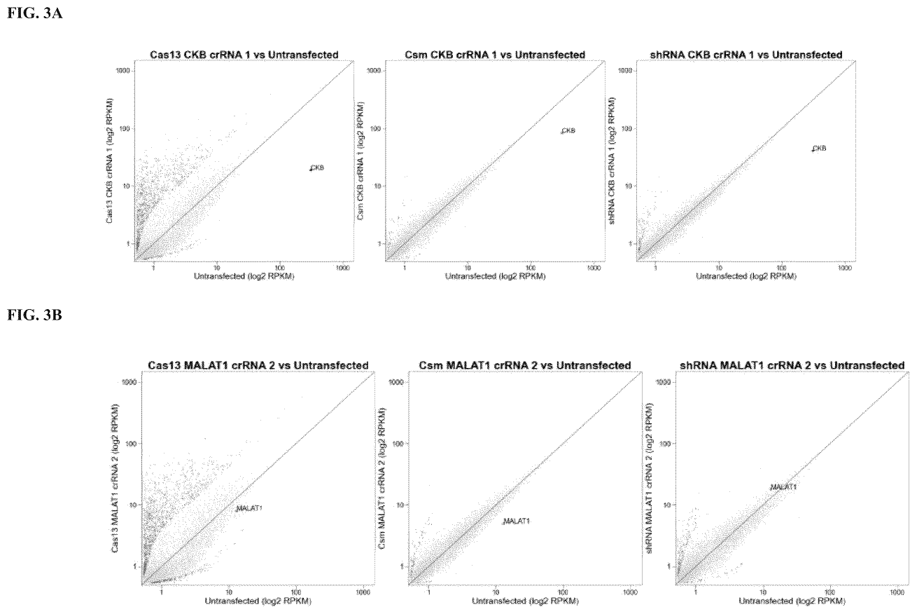

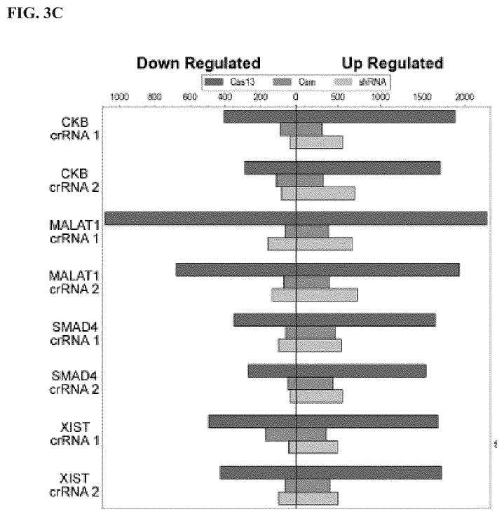

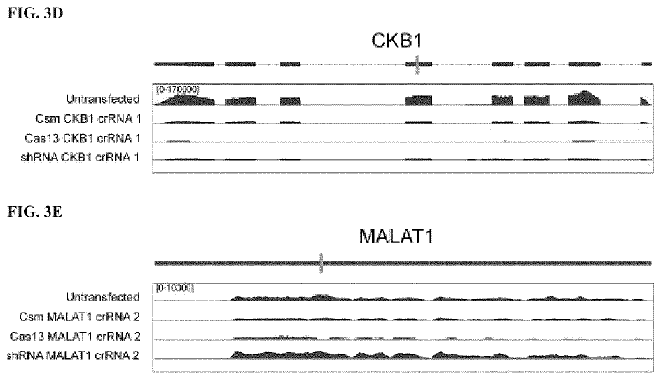

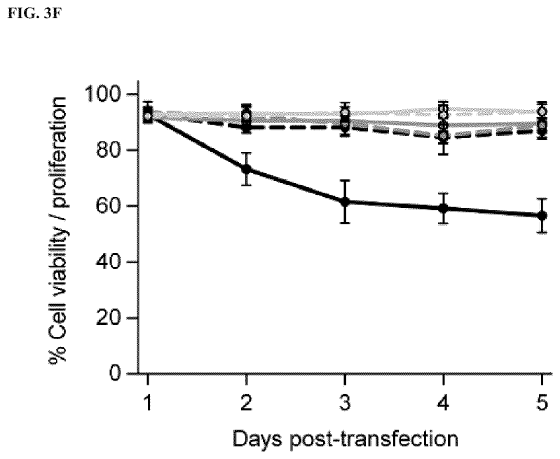

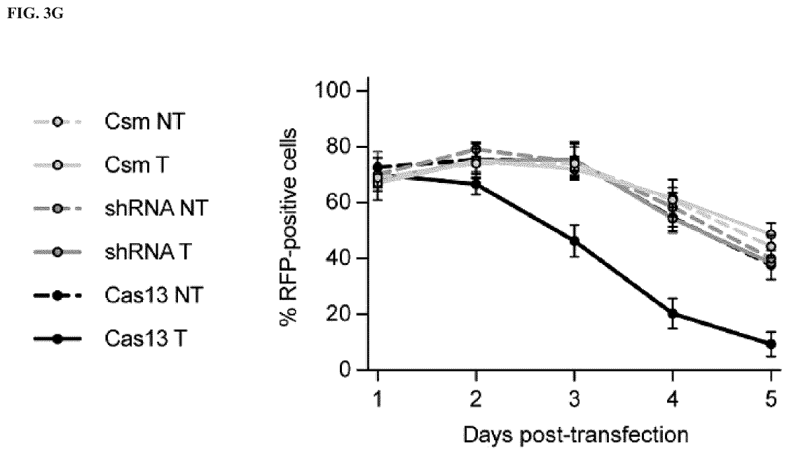

RNA-sequencing was performed to examine potential off-target effects of Csm-mediated KD in cells. For comparison with other established KD technologies, RNA-seq was also performed for Cas13 (RfxCas13d) and RNAi (shRNA)-mediated KD (Wei et al. 2021; Bofill-De Ros and Gu 2016; Wessels et al. 2020). XIST, MALAT1, CKB, or SMAD4 was depleted for 48 hr using Csm, Cas13, or shRNA using crRNAs/shRNAs targeting the same complementary sequence for each transcript. Scatterplots comparing differential transcript levels between Csm-treated and untreated samples showed significant KD of the target transcript (CKB, MALAT1 shown) with few other differentially expressed genes (≥2-fold change, indicated in red) (FIG. 3A,B). Cas13-treated samples showed significant KD of the target but with thousands of differentially expressed off-target genes. shRNA-treated samples showed variable KD depending on whether the target was cytoplasmic (CKB) or nuclear (MALAT1), with few other differentially expressed genes. Similar trends were seen for all four target transcripts and both crRNAs/shRNAs per transcript (FIG. 3C). Examination of RNA-seq read coverage confirmed that target KD was transcript-wide and not only localized near the Csm cleavage sites—unsurprising given exonucleotic RNA degradation pathways in mammalian cells (Houseley and Tollervey 2009) (FIG. 3D,E). Hence, unlike Cas13, Csm- and shRNA-mediated RNA KD has few off-target effects in human cells.

Other RNA-targeting CRISPR-Cas systems such as Cas13 may suffer from severe cytotoxic effects due to inherent trans-cleavage activity of the Cas effector (Q. Wang et al. 2019; Ozcan et al. 2021; Ai, Liang, and Wilusz 2022; Tong et al. 2021; Shi et al. 2021). Type III systems do not exhibit such trans-activity and are thus poised to offer robust RNA KD without such toxicity. To check this, cell proliferation/viability was tracked using the WST-1 assay across a time-course after transfecting cells with Csm, Cas13, or shRNA constructs (FIG. 3F). Whereas Cas13-treated cells exhibited a significant decrease in proliferation/viability, Csm- or shRNA-treated cells were unaffected. This decrease in proliferation/viability was accompanied by a more rapid decrease over time in the proportion of RFP-positive (transfected) cells for the Cas13-treated population compared to the Csm- or shRNA-treated population (FIG. 3G). Taken together, these results suggest that use of Cas13, but not Csm or shRNAs, may cause pronounced toxicity in cells in this experimental system.

FIG. 3A-3G. RNA knockdown with minimal off-targets or cytotoxicity. A,B. Scatterplots showing differential transcript levels between Csm, Cas13, or shRNA-treated cells targeting CKB (A) or MALAT1 (B) versus untreated cells. Target transcript is indicated; red dots indicate differentially regulated off-targets (≥2-fold change). C. Quantification of significantly up- or down-regulated genes (≥2-fold change) for each sample. D,E. RNA-seq read coverage across target transcripts, CKB (D) or MALAT1 (E), in Csm, Cas13, or shRNA-treated cells. Orange bar indicates location of crRNA/shRNA target sequence. F. Relative cell viability and proliferation (normalized to untransfected cells) of HEK293T cells at the indicated times post transfection with the indicated targeting (T) or non-targeting (NT) plasmids, measured by WST-1 assay. G. Relative abundance of RFP-positive HEK293T cells (normalized to untransfected cells) at the indicated times post transfection with the indicated targeting (T) or non-targeting (NT) plasmids, measured by flow cytometry.

Live-Cell RNA Imaging without Genetic Manipulation

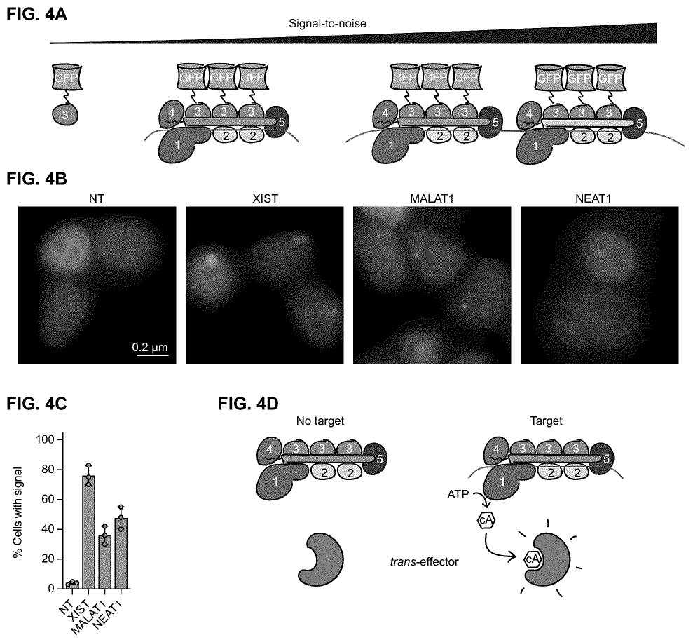

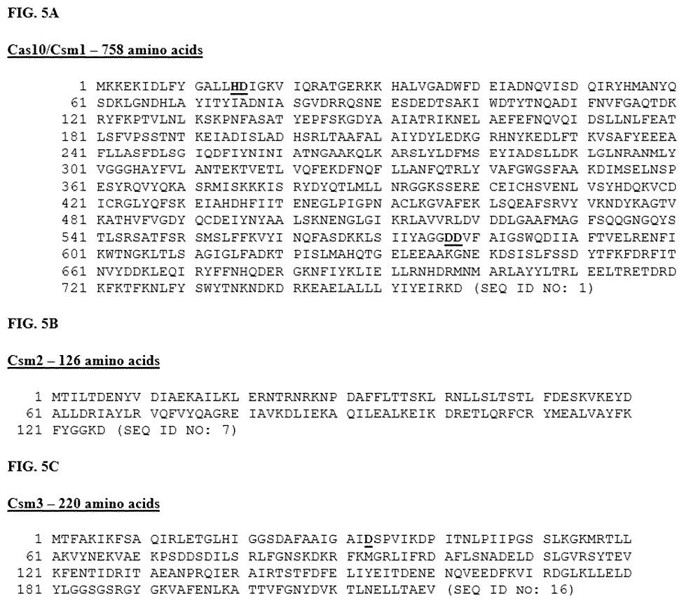

Tracking RNA in live cells remains a difficult task, often requiring genetic insertion of aptamer sequences into the RNA target, which is both laborious and potentially disruptive to RNA function and/or regulation (George et al. 2018). Fluorescently tagged programmable RNA-binding proteins such as catalytically inactivated Cas13 have recently been adopted for such purposes (Abudayyeh et al. 2017; H. Wang et al. 2019; Yang et al. 2019). It was asked whether the Csm complex could similarly be used to track RNA targets in live cells. To test this, GFP was fused to the C-terminus of catalytically inactivated Csm3 in the vector (FIG. 4A). This super-stoichiometric subunit (≥3 per complex) was chosen in order to increase the signal-to-noise ratio of complexed, target-bound Csm over unbound, unassembled subunits (FIG. 4B). To visualize XIST RNA, its “Repeat A” region was targeted with a single crRNA predicted to bind 8 times per transcript, further increasing signal. Whereas a non-targeting control crRNA led to only background nuclear fluorescence, the XIST-targeting crRNA led to a strong cloud-like signal in most cells (FIG. 4C and FIG. 4D), characteristic of XIST RNA and phenocopying what was previously observed by XIST RNA FISH (FIG. 2E). Similar results were obtained for MALAT1 and NEAT1 RNAs, even with crRNAs predicted to bind only once per target transcript (FIG. 4C and FIG. 4D). Multiplexing several crRNAs against the same target will likely further improve signal-to-noise, especially for targets of low abundance. Thus, fluorescently-tagged Csm can be used for easy visualization of RNA in living cells.

FIG. 4A-4D. Live-cell RNA imaging without genetic manipulation. A. Diagram showing Csm-GFP fusion. Signal-to-noise increases from left to right, from unassembled Csm3, to target-bound Csm complexes, to multiplexed target-bound complexes. B. Live-cell fluorescent imaging of HEK293T cells transfected with Csm-GFP protein complex and the indicated crRNAs. C. Quantification of (B). 100 transfected cells were counted for each condition. D. Diagram showing RNA sequence-dependent activation of downstream effectors by the Csm complex.

### Example 2 (Update to Example 1)

### Methods

See Example 1 above

### Results

### An All-In-One Type III CRISPR-Cas System in Human Cells

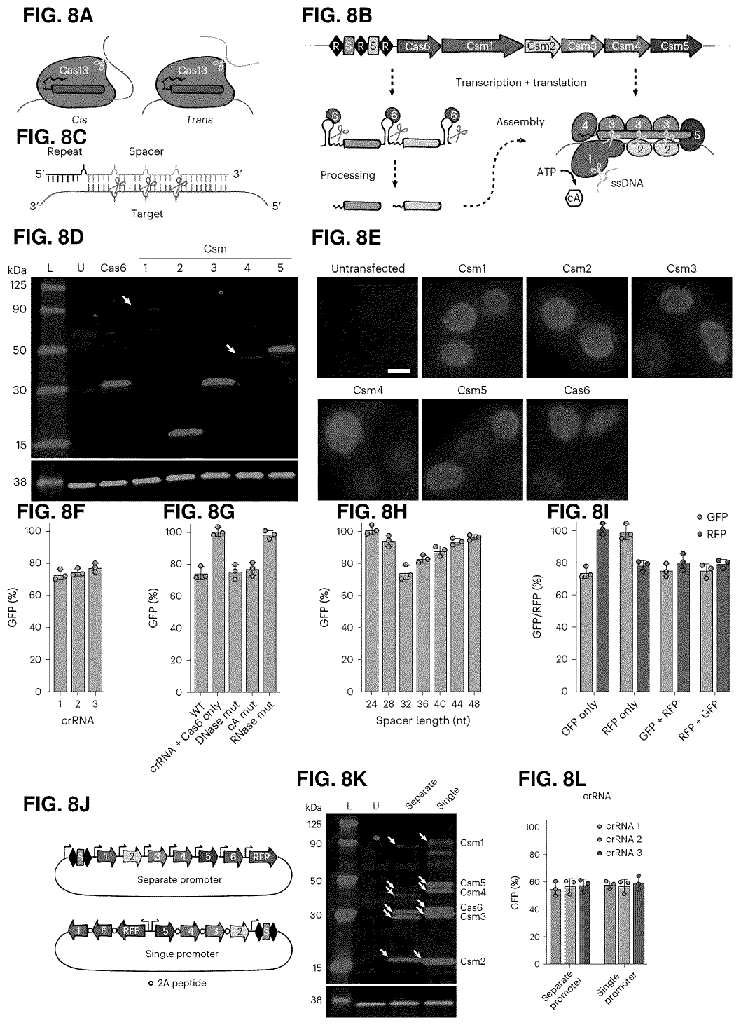

The Type III-A Csm complex from Streptococcus thermophilus (“Sth”) was chosen for several reasons as follows: (1) it has been extensively characterized biochemically, structurally and in bacteria, (2) it functions optimally at 37° C., (3) it has been demonstrated to work in zebrafish embryos and human cell culture upon ribonucleoprotein (RNP) delivery and (4) it has fewer components than the analogous type III-13 Cmr complex. Proper expression of each individual protein component (Csm1-5 and Cas6) was verified in immortalized human embryonic kidney (HEK293T) cells. Proteins were human codon optimized, N-terminally FLAG-tagged for detection and expressed from a cytomegalovirus promoter. While RNAi operates in the cytoplasm where mRNAs mainly reside, Cas6 and each Csm component was localized to the nucleus through the addition of an N-terminal SV40 nuclear localization signal so as to target nuclear RNAs and pre-mRNAs before export. Following transient transfection, Western blot (FIG. 1d) and immunofluorescence staining (FIG. 8e) verified proper size, expression and nuclear localization of each protein.

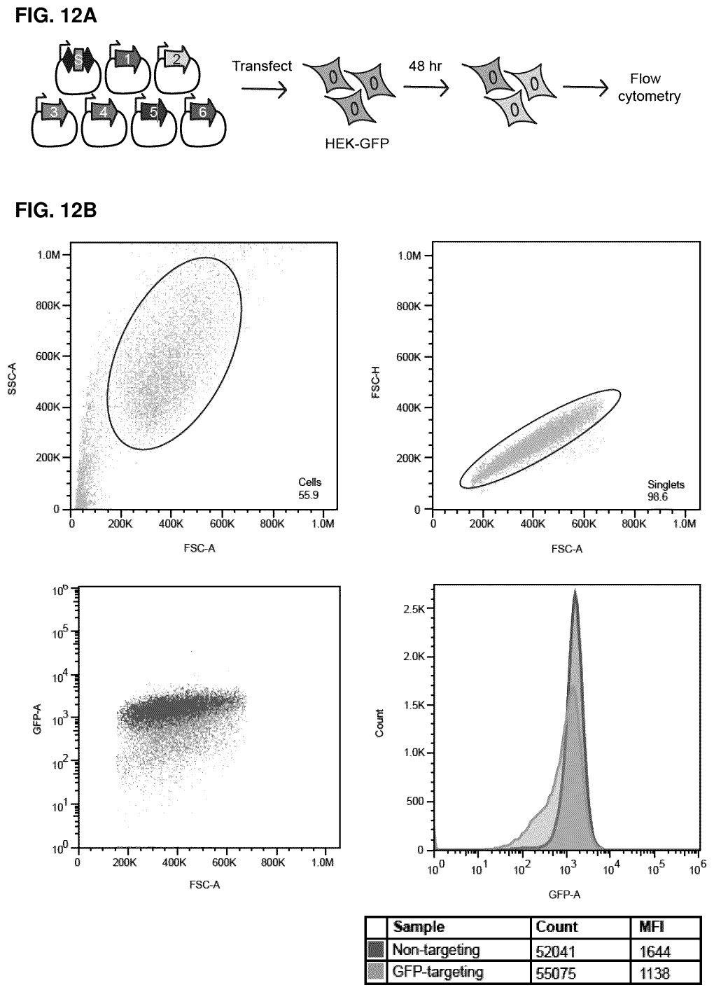

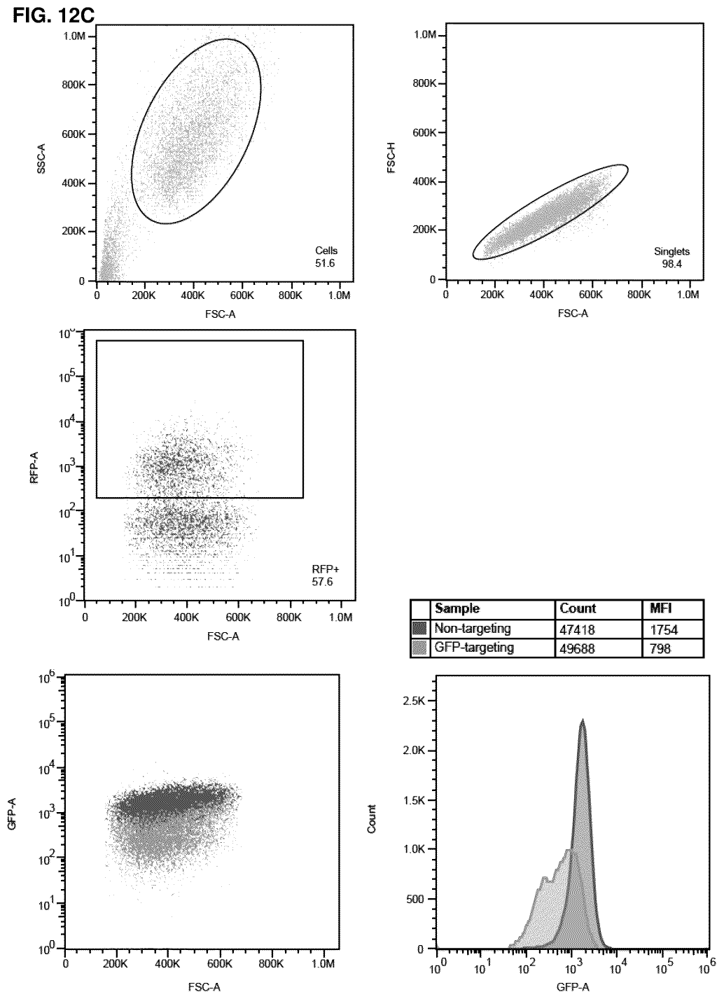

To test the system, enhanced green fluorescent protein (eGFP; henceforth ‘GYP’) mRNA was targeted in a GFP-expressing HEK293T cell line. Seven plasmids individually expressing Csm1-5, Cas6 and either a GFP-targeting or nontargeting crRNA from a U6 promoter were cotransfected into cells, and GFP fluorescence assayed by flow cytometry 48 h post transfection (FIG. 12a). Note that this strategy does not allow any means to select cells into which all plasmids were successfully delivered and will thus under-report KD efficiency. GFP KD was calculated by dividing the mean fluorescence intensity (NMI) of cells transfected with the GFP-targeting crRNA by that of cells transfected with the nontargeting crRNA (FIG. 12b). Approximately 25% KD was observed using any of three crRNAs targeting different regions of the GFP ORE (FIG. 8f). Notably, no KD was seen after transfecting the GFP-targeting crRNA and its processing factor (Cas6) alone (FIG. 8g), indicating that KD was not due to an antisense RNA effect. Furthermore, whereas ablating DNase (H15A, DMA) or cA synthase (D577A, D578A) activities in Csm1 did not noticeably affect GFP KD, ablating RNase activity (D33A) in Csm3 abolished it (FIG. 8g), indicating RNase activity is responsible for the observed KD.

Next, crRNA parameters were examined. Naturally occurring spacers for Sth Csm crRNAs range from ˜30 to 45 nt in length, although in vitro, spacers as short as 27 nt are sufficient to trigger all three catalytic activities. The GET-targeting spacer length was varied from 24 nt to 48 nt in increments of four and assayed GFP KD. A length of 32 nt yielded the highest KD for the crRNA tested (FIG. 8h), with little to no KD seen for lengths ≤28 nt, and diminishing KD seen for lengths ≥36 nt, A more large-scale analysis must be performed to determine whether optimal spacer length differs from sequence to sequence. Next, the potential to multiplex crRNAs against several targets was examined. Two crRNAs were encoded within a single array—one targeting GET and the other targeting mCherry (henceforth ‘red fluorescent protein (RFP)’)—and examined KD of GFP and RFP in a HEK293T cell line expressing both (FIG. 8i). Approximately 25% KD was achieved for both GFP and RFP regardless of the order of crRNAs in the array (GFP-RFP or RFP-GFP), comparable to KD efficiency when targeting GFP or REP alone. Together, these results demonstrate broad multiplexing capability for the Csm system.

With the Csm system up and running, delivery was simplified by consolidating all components into a single vector. For this, the following two approaches were pursued concurrently: (1) expression of each protein from separate promoters or (2) expression of all proteins from a single bidirectional promoter separated by 2A peptides (FIG. 8j). RFP was also included in the plasmid backbone to allow identification of transfected cells and thus more accurate measurement of KD efficiency (FIG. 12c). After reconfirming proper expression of all protein components by Western blot for both plasmids (FIG. 8k), both strategies (after optimizing the order of proteins in the single-promoter arrangement) led to ˜50% GFP KD in transfected cells (FIG. 8I). In summary, the single-promoter design is well-equipped for promoter-swapping and thus use in specific cell types or other eukaryotic systems, while the modular design of the separate-promoter vector allows for easy swapping or modification of individual Csm components. All further experiments were performed using the separate-promoter vector.

### Robust KD of Endogenous Nuclear and Cytoplasmic RNAs

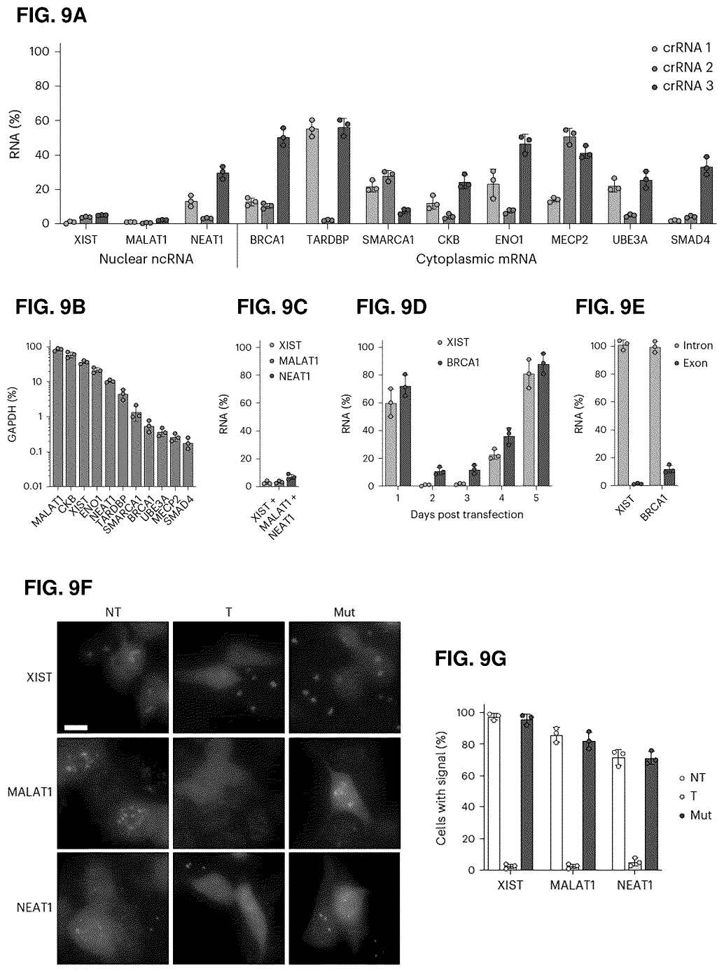

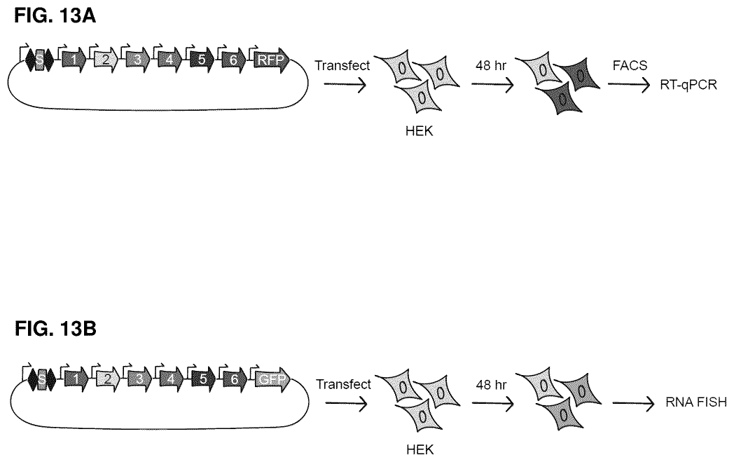

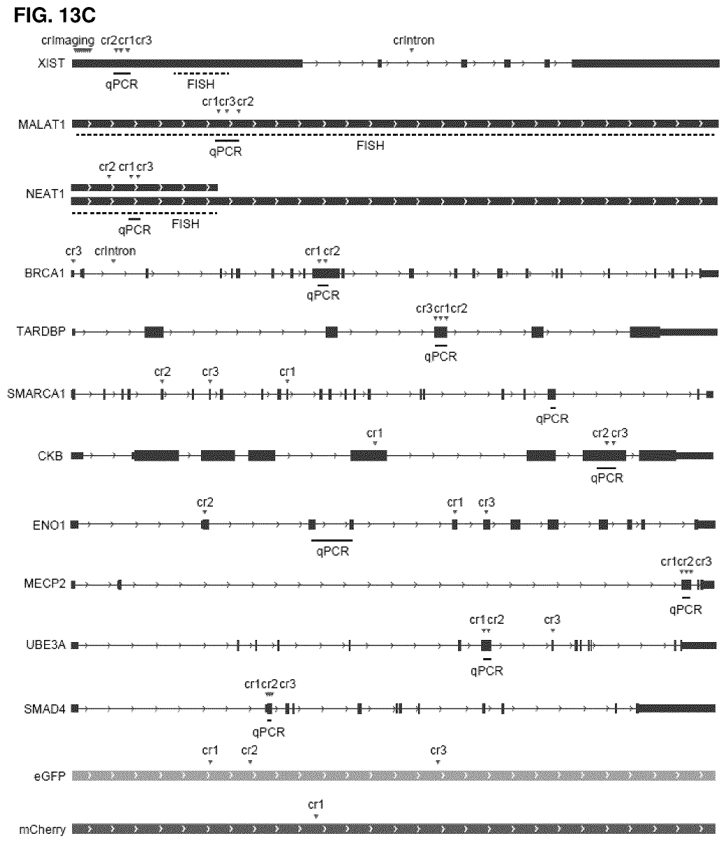

Thus far, Csm had been used to KD highly overexpressed, heterologous GFP/RFP transgenes and assayed KD at the protein level (half-life >24 h), which may not accurately reflect abundance at the RNA level. It was sought to target endogenous transcripts and assay RNA KD directly. A panel of three nuclear noncoding RNAs (XIST, MALAT1 and NEAT1) and eight cytoplasmic mRNAs (BRCA1, TARDBP, SMARCA1, CKB, ENO1, MECP2, UBE3A and SMAD4) (FIG. 9a) of varying abundances (FIG. 9b) was targeted, testing three individual crRNAs for each. HEK293T cells were transfected with all-in-one vector, transfected (REP-positive) cells were isolated by FACS after 48 h, total cell RNA was extracted and RNA KD was assayed by RT-gPCR (FIGS. 12c and 13a,c). Surprisingly, >90% KD was achieved for all eleven RNAs with at least one crRNA, compared to nontargeting crRNA control (FIG. 9a). It was also confirmed that multiplexed KD for three of the RNAs (XIST, MALAT1 and NEAT1) (FIG. 9c) was possible. These results demonstrate Csm to be a highly robust and efficient RNA KD tool for not only cytoplasmic but also nuclear RNAs, which are typically recalcitrant to KD by conventional RNAi methods.

To examine KD kinetics, the above RT-qPCR experiment was repeated for two of the RNA targets (XIST and BRCA1) across a 5-d time course. KD peaked d post transfection and waned thereafter (FIG. 9d), as might be expected from the transient transfection method used to deliver Csm into cells. KD efficiency of crRNAs targeting intronic versus exonic regions was also compared for the same two RNAs (FIG. 9e). Targeting introns did not lead to any noticeable reduction in the mature transcript, possibly because introns are excised from the pre-mRNA more rapidly than they are cleaved by Csm.

To corroborate RNA KD with an orthogonal method, RNA fluorescent in situ hybridization (FISH) was performed for all three nuclear noncoding RNAs, which are easily visualized and display characteristic morphologies. HEK293T cells were transfected with Csm plasmid carrying a GFP reporter (to identify transfected cells) and either a targeting or nontargeting crRNA and assayed by RNA FISH after 48 h (FIG. 13b,c). XIST, MALAT1 and NEAT1 were all readily detected when delivering a nontargeting crRNA control (FIG. 9f,g). By contrast, use of a single targeting crRNA abolished all visible signals for each target RNA in transfected (GFP-positive) cells, whereas signal was still detected in untransfected (GFP-negative) cells. For further validation, delivery of targeting crRNA with catalytically inactivated Csm (RNase mut) fully restored the detection of each target RNA. Thus, robust KD of endogenous transcripts was demonstrated using active Csm complexes by both molecular and microscopy-based techniques.

RNA KD with Minimal Off-Targets or Cytotoxicity

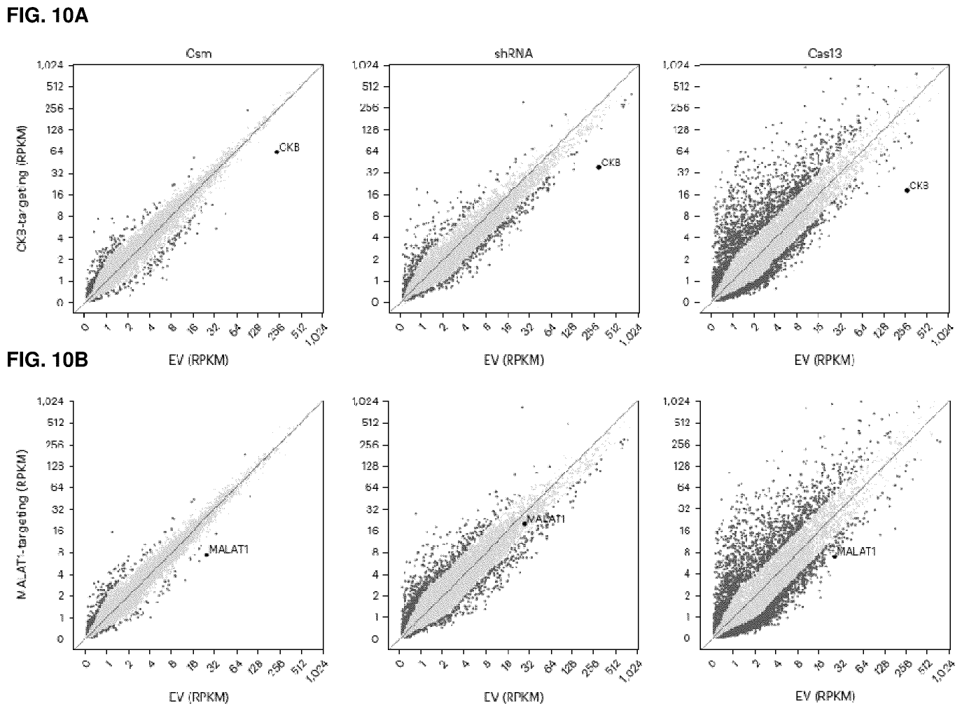

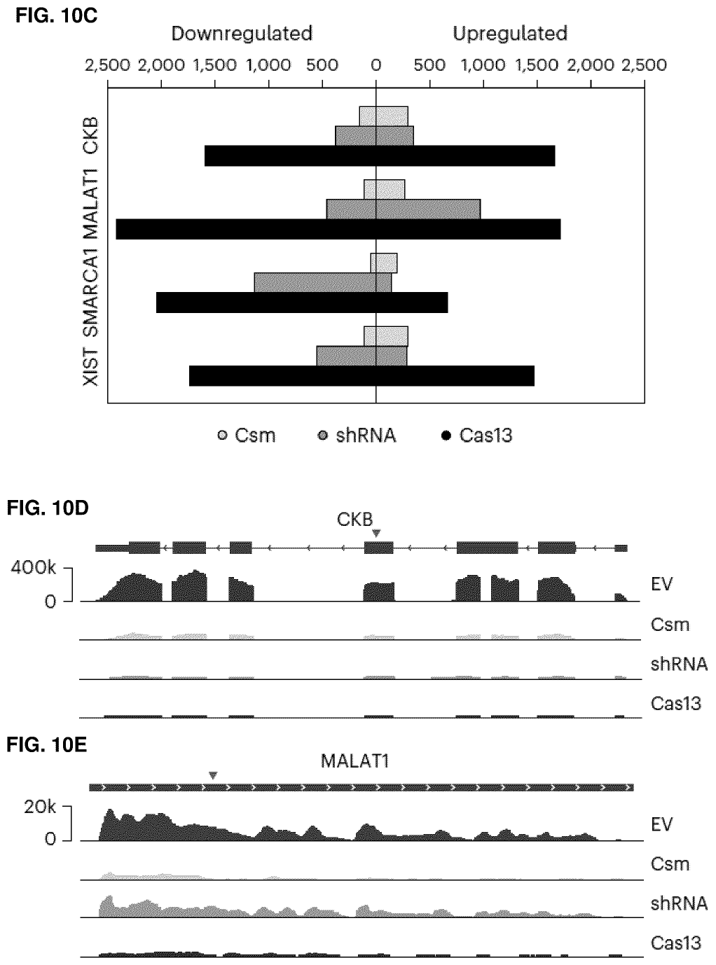

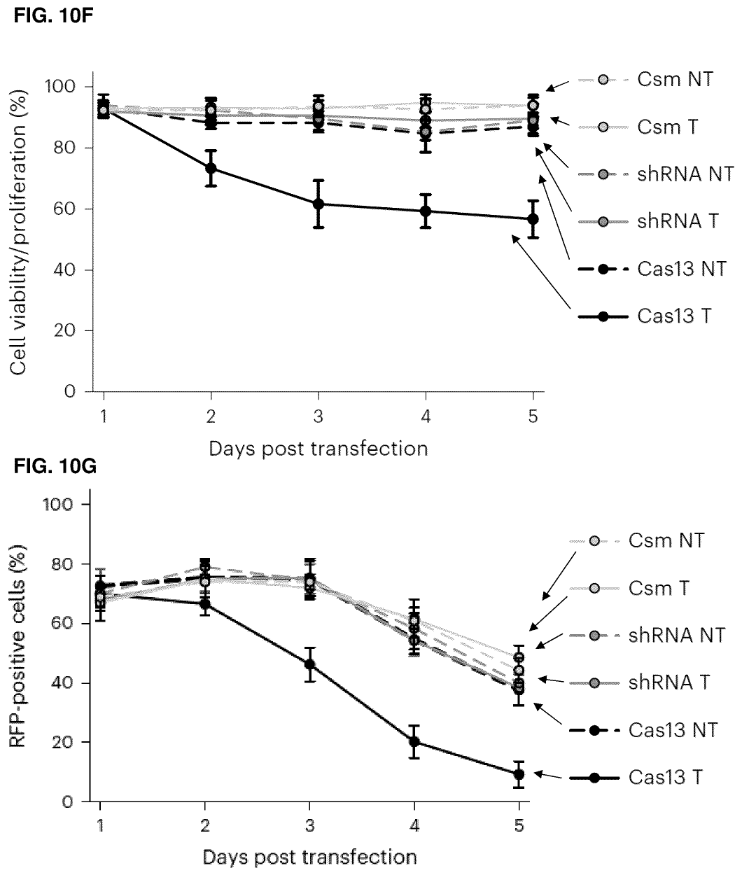

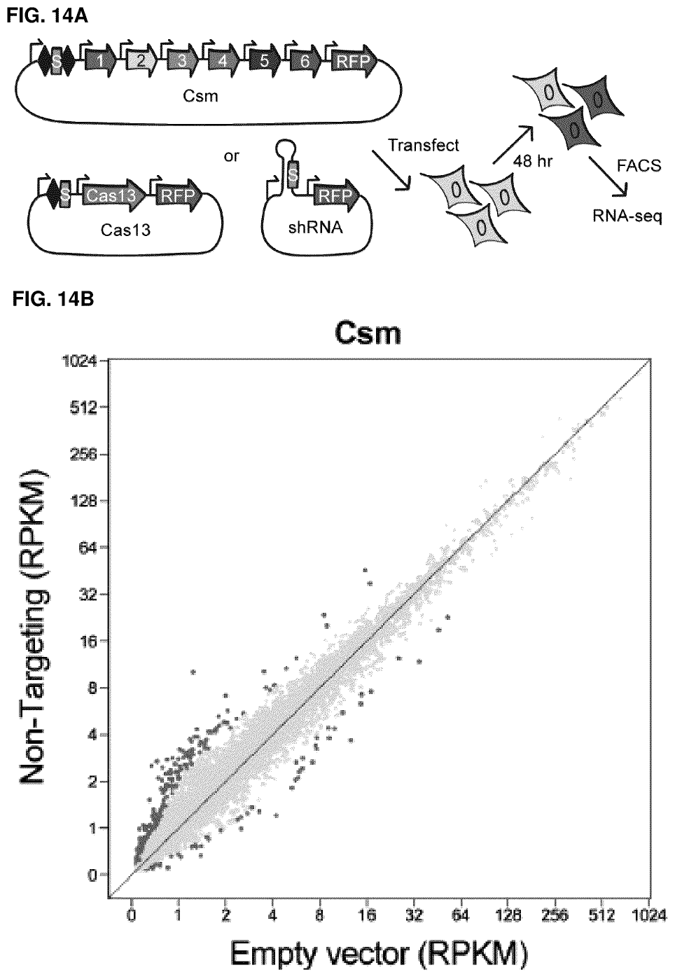

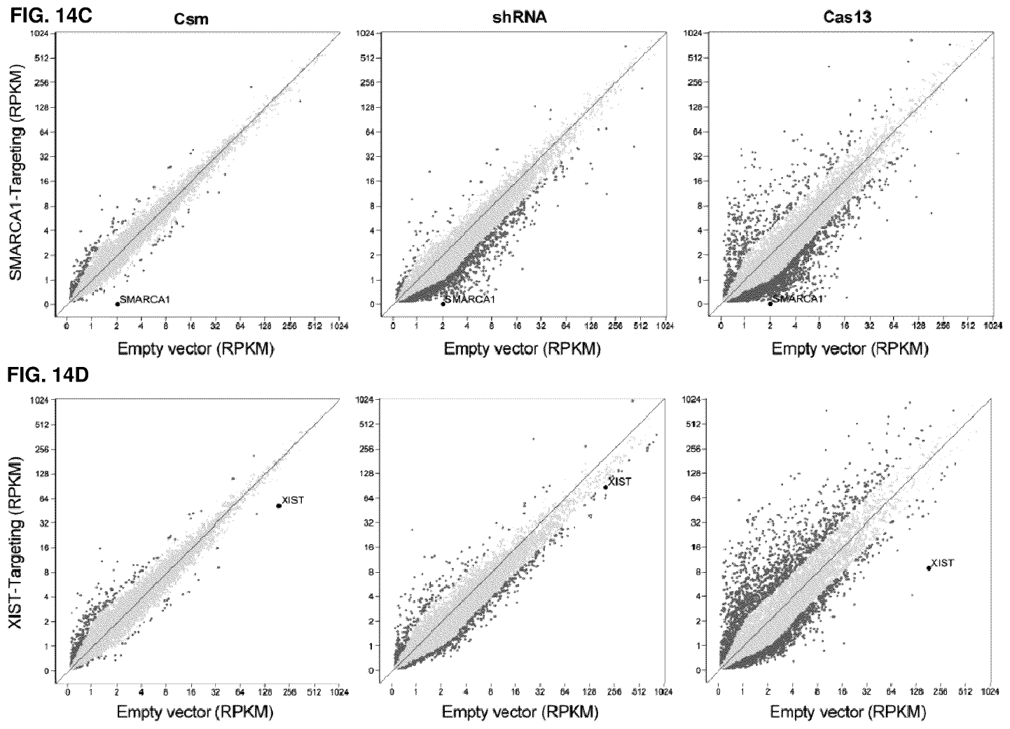

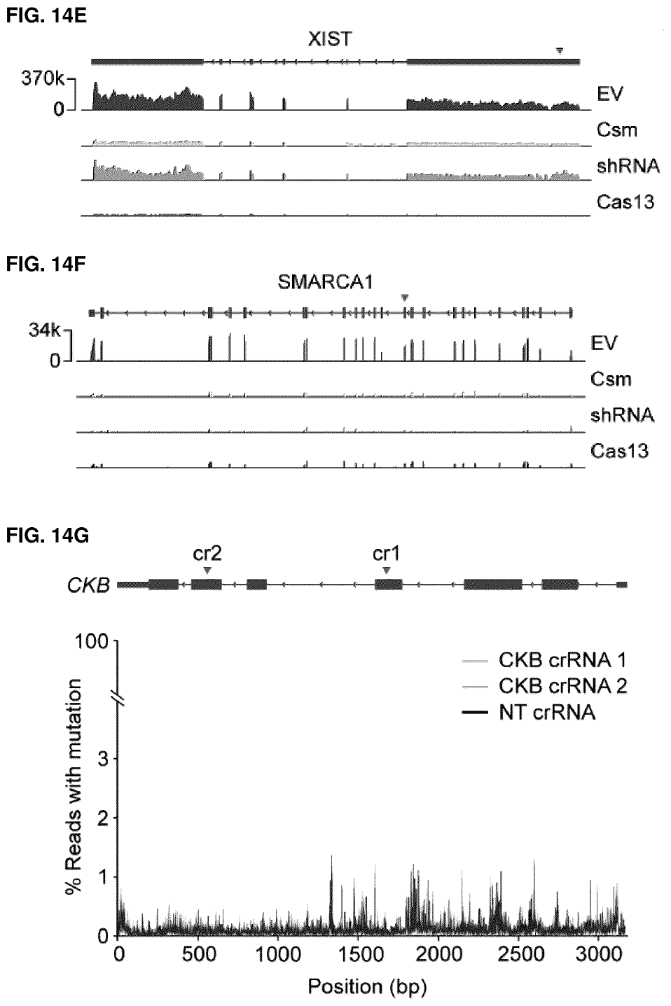

Next, RNA sequencing (RNA-seq) was performed to examine the potential off-target effects of Csm-mediated KD in cells. For comparison with other established KD technologies, RNA-seq was also performed for Cas13 (RfxCas13d) and RNAi (short hairpin RNA (shRNA))-mediated KD using crRNAs/shRNAs targeting the same complementary sequence. KD was performed for 48 h, after which transfected cells were enriched by FACS and sequenced (FIG. 14a). Scatterplots comparing transcript levels between nontargeting crRNA and empty vector (EV) control samples for Csm revealed few upregulated or downregulated transcripts (defined as ≥2-fold change, indicated in red) (FIG. 14b), suggesting Csm expression itself does not substantially perturb the cellular environment. When targeting CKB, MALAT1, SMARCA1 or XIST, Csm-mediated KD led to significant depletion of the target transcript with few other altered transcripts (FIG. 10a,b and FIG. 14c,d). Meanwhile, Cas13 samples showed significant KD of the target transcript while also affecting hundreds of nontarget transcripts. shRNA samples showed variable KD depending on whether the target was cytoplasmic (CKB, SMARCA1) or nuclear (MALAT1, XIST), with an intermediate amount of altered nontarget transcripts. Similar trends were seen for all four targets (FIG. 10c). Examination of RNA-seq read coverage across the target confirmed depletion was transcript-wide and not only localized near the site of Csm cleavage (red arrow), likely due to cellular exonucleotic degradation pathways (FIG. 10d,e and FIG. 14e,f). It, was also examined whether Csm-mediated RNA-targeting induces any collateral changes at the DNA level due to its separate DNase activity. DNA-sequencing across the entire CKB locus did not reveal any noticeable differences between targeting and nontargeting samples at a sequencing depth of ˜1 million reads (FIG. 14g). Alternatively, DNase activity can be removed without affecting RNase activity (FIG. 8g). Hence, Csm-mediated RNA KD shows minimal off-target effects in human cells.

Other RNA-targeting CRISPR-Cas systems such as Cas13 suffer from severe cytotoxic effects due to inherent trans-cleavage activity. Type III systems do not exhibit trans-activity and are thus poised to offer robust RNA KD without toxicity. To check this, cell proliferation/viability was tracked using the WST-1 assay across a time course after transfecting cells with targeting or nontargeting Csm, Cas13 or shRNA constructs (FIG. 10f). Whereas cells that received targeting Cas13 constructs exhibited a significant decrease in proliferation/viability, those that received Csm or shRNA constructs were unaffected. This decrease in proliferation/viability by WST-1 assay was also seen by a more rapid decrease over time in the proportion of RFP-positive (transfected) cells within the targeting Cas13-treated population compared to the Csm- or shRNA-treated population (FIG. 10g). Taken together, these results suggest that, unlike Cas13, Csm-mediated KD has minimal toxicity in cells.

Live-Cell RNA Imaging without Genetic Manipulation

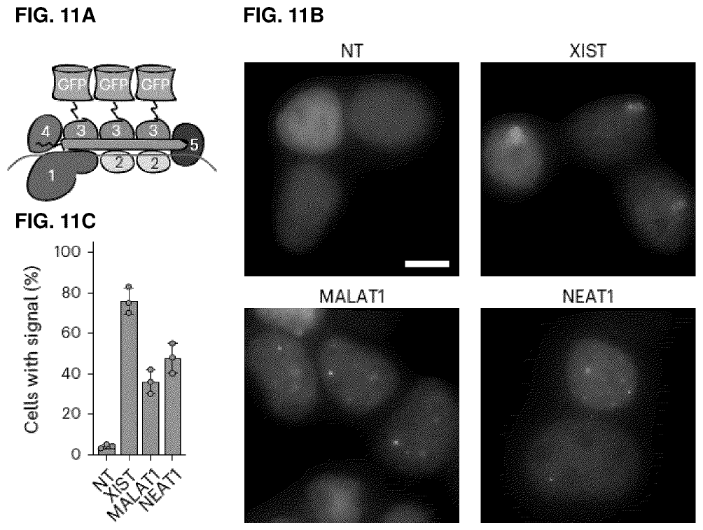

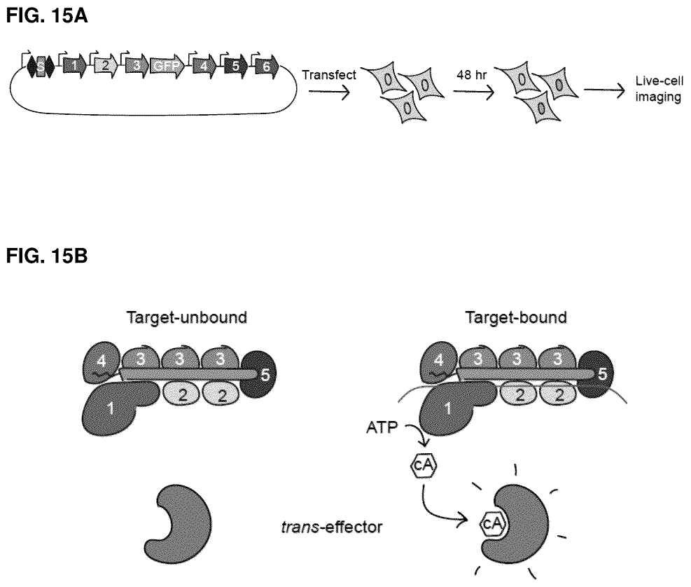

Tracking RNA in live cells remains a difficult task, often requiring genetic insertion of aptamer sequences into the target, which is both laborious and potentially disruptive to RNA function and/or regulation. Fluorescently tagged programmable RNA-binding proteins such as catalytically inactivated Cas13 have recently been adopted for such purposes. Whether the Csm complex could similarly be used to track RNA targets in live cells was next asked. To test this, GFP was fused to catalytically inactivated Csm3 (FIG. 11a), the most abundant Csm subunit (≥3 per complex), thereby allowing multivalent display. To visualize XIST RNA, a repetitive region was targeted with a single crRNA predicted to bind eight times per transcript, allowing increased signal. HEK293T cells were transfected with Csm-GTP plasmid and assayed by live-cell fluorescence microscopy after 48 h (FIG. 15a). Whereas a nontargeting control crRNA led to only background nuclear fluorescence, the XIST-targeting crRNA led to a strong cloud-like signal in most cells (FIG. 11b,c), phenocopying what was observed by XIST RNA FISH (FIG. 9f). Using the same approach, MALAT1 and NEAT′ transcripts were visualized, even with crRNAs predicted to bind only once per target (FIG. 11b,c). Multiplexing several crRNAs against the same target will likely further improve signal over background, especially for lower abundance transcripts. Thus, fluorescently tagged Csm can be used for easy visualization of RNA in living cells.

### Discussion

It was shown in the experiments here that the type III-A Csm complex (e.g., from S. thermophilus) is a powerful tool for eukaryotic RNA KD. Both nuclear noncoding RNAs and cytoplasmic mRNAs were knocked down with high efficiency (90-99%) and specificity (˜10-fold fewer off-targets than Cas13), outperforming competing RNA KD technologies. More notably, KD was not accompanied by detectable cytotoxicity, unlike Cas13-based methods that suffer from inherent trans-cleavage activity.

Recently, StCsm was shown to be effective at depleting GFP or viral RNA upon delivery of bacterially purified RNP into zebrafish embryos or human cells, respectively (Fricke, T. et al. Targeted RNA knockdown by a type 3 CRISPR-Cas complex in zebrafish. CRISPR J. 3, 299-313 (2020); and Lin, P. et al. Type 3 CRISPR-based RNA editing for programmable control of SARS-CoV-2 and human coronaviruses. Nucleic Acids Res. 50, e47 (2022). RNP delivery of multisubunit CRISPR-Cas effectors is not ideal for several reasons as follows: (1) it is often difficult and short-lived compared to DNA-delivery methods, (2) the RNP may be unstable and prone to disassembly and (3) for every new crRNA, the entire RNP must be repurified from bacteria or reconstituted from individually purified subunits in the proper ratio. These hurdles were overcome here by encoding all necessary parts in a single deliverable plasmid.

More recently, a single-protein type III effector, Cas7-11, was characterized and used for RNA KD in eukaryotes. This effector is interesting from an evolutionary and structural standpoint in that it appears to have arisen from fusion of the canonical type III subunits into one large polypeptide. While simpler to introduce into eukaryotes, Cas7-11's demonstrated RNA KD efficiency was only 25-75% for most targets (without enriching for transfected cells), making it somewhat less practical as a tool.

A key advantage of the approach here over RNAi is the ability to target transcripts in the nucleus. >95% KD was achieved for three biologically significant nuclear ncRNAs (XIST, MALAT1 and NEAT1). Nuclear RNAs are notoriously difficult to KD, often requiring expensive chemically modified antisense oligos to direct RNase H-mediated cleavage. However, the increased stability of these oligos often leads to unexpected off-target hybridization and cytotoxic effects. Aside from long ncRNAs, nuclear targeting will likely prove useful for the study of other mRNA species such as eRNAs, tRNAs, rRNAs, circRNAs, miRNAs and snoRNAs. For instance, targeting introns containing miRNA or snoRNA clusters will facilitate their degradation before processing/maturation and targeting particular exons will likely alters the abundance of mRNA splice isoforms.

Another advantage of the system here is its ease of multiplexing. Multiple spacers can be cloned into the CRISPR array and processed into individual crRNAs by Cas6. This allows for pooled screening, either by encoding crRNAs against multiple targets at once or encoding multiple crRNAs against the same target. The latter may enable robust KD on the first try without the need to individually screen multiple crRNAs against a target. An unexpected observation was the titratable nature of KD with increasing spacer length. This will likely facilitate easy tunability of KD (rather than all-or-none) when studying concentration-dependent effects of gene products.

Csm-mediated RNA KD appears robust. Significant KD was achieved for nearly all targets tested, with at least one of three crRNAs per target yielding >90% KD. Because, like other RNA-targeting CRISPR-Cas systems, Csm does not have any PAM requirement for target site selection, the only criteria used were that the target be a unique sequence in the human transcriptome and the spacer avoid stretches of ≥5 consecutive Ts, which might cause premature Pol III transcriptional termination within the crRNA sequence. The observed variability in KD efficiency from one crRNA to another may in part be explained by differences in target site accessibility due to local RNA secondary structure or protein occupancy.

The work here showed that fluorescently tagged, catalytically inactivated Csm can be used for live-cell RNA visualization. By fusing GFP to the most abundant subunit (Csm3), multivalent display (≥3×GFP per complex) was achieved, which offers advantages over single-subunit effectors such as Cas13. Beyond GFP, other proteins of interest can be fused to the various Csm subunits to achieve assembly or tethering at a desired stoichiometric ratio. Thus, as a multisubunit complex, Csm offers the benefits of split-protein systems without the engineering effort. Catalytically inactivated Csm is also useful for disrupting RNA structural motifs or RNA-protein interactions without manipulation at the DNA level.

By bringing type III systems to eukaryotes, the way has now been paved for co-introduction of related trans-effectors that can be activated in an RNA sequence-dependent manner (see, e.g., FIG. 15b). This system can be used for RNA diagnostics, screens and synthetic circuits in vivo.

### Sequences

TABLE 1
Sequences used

| | | SEQ ID | | SEQ ID |
|---|---|---|---|---|
| Target | qPCR primer F | NO | qPCR primer R | NO |
| XIST | GTTGTATCGGGAGGCAGTAAGA | 71 | GAAAAGCACACAGCAAAGACAAAGA | 83 |
| | ATCATCTTT | | GGC | |
| MALAT1 | ACTAGCATTAATTGACAGCTGA | 72 | GCTACCTTCATCACCAAATTGCACTC | 84 |
| | CCCAGG | | G | |
| NEAT1 | GCTTAGGAGGAGGAAGTTCTCC | 73 | CTCCATCTGCAAGCTCCATCTACAAG | 85 |
| | AATGT | | | |
| BRCA1 | TACATCAGGCCTTCATCCTGAG | 74 | ACAATTAGGTGGGCTTAGATTTCTAC | 86 |
| | GATTTTATC | | TGACTACTA | |
| TARDBP | GTCAAGAAAGATCTTAAGACTG | 75 | CTTAGAATTAGGAAGTTTGCAGTCAC | 87 |
| | GTCATTCAAAGGG | | ACCATC | |
| SMARCA1 | GATAAACCAGTCAAATCTAAAC | 76 | GATACAAGGCTCCATTTCATCAGTTG | 88 |
| | TGGGGAGCA | | CC | |
| CKB | TTAAGCACCTCCGAGAACTTCT | 77 | TTGAAACTCTCTTCAAGTCTAAGGAC | 89 |
| | CATGC | | TATGAGTTCA | |
| ENO1 | GGAACTCATTAATATACTTAAT | 78 | CTTCAACTGGTATCTATGAGGCCCTA | 90 |
| | GGGTCTGGAGACG | | GAG | |
| MECP2 | GGAAGAAAAGTCAGAAGACCA | 79 | TTGATCAAATACACATCATACTTCCC | 91 |
| | GGACC | | AGCAGAG | |
| UBE3A | ATATTGATGCCATTAGAAGGGT | 80 | CTTTGCAAAATAATGGCAAAGCCATT | 92 |
| | CTACACCAGAT | | TCCAG | |
| SMAD4 | ATGGACAATATGTCTATTACGA | 81 | CTGAAGCCTCCCATCCAATGTTCTCT | 93 |
| | ATACACCAACAAGTAATG | | | |
| GAPDH | CCAGAACATCATCCCTGCCTCT | 82 | GGAAATGAGCTTGACAAAGTGGTCG | 94 |
| | ACTG | | TTG | |

TABLE 2
Sequences used

| | Genomic PCR | Genomic PCR |
|---|---|---|
| Target | primer F | primer R |
| CKB | AATGGAATGAATGGGC | CTTGTCCCATCTC |
| | TATAAATAGCCGCC | ACAGAAGGCGAG |
| | (SEQ ID NO: 95) | (SEQ ID NO: 96) |

TABLE 3
Sequences used

| Target | Csm crRNA 1 | Csm crRNA 2 | Csm crRNA 3 |
|---|---|---|---|
| XIST | GCCACTTGAACACTGCG | TTGGACAACCTAACAAAGCAC | CGCACATGTCCACCACCATGC |
| | ACAGAACTGGATCCG | AGCCCGCCATG | TAACCACTTAA |
| | (SEQ ID NO: 97) | (SEQ ID NO: 111) | (SEQ ID NO: 123) |
| MALAT1 | AGCTTCCTTCACCAAATC | GCCGCCTGCTACCTTCATCACC | CCTAGCTTCACCACCAAATCG |
| | GCACTGGCTCCTGG | AAATTGCACT | TTAGCGCTCCT |
| | (SEQ ID NO: 98) | (SEQ ID NO: 112) | (SEQ ID NO: 124) |
| NEAT1 | CCGGATGCATCTGCTGTG | CACCATTACCAACAATACCGA | GAAGATGCAGCATCTGAAAAC |
| | GACTTTTTAAGATT | CTCCAACAGCC | CTTTACCCCAG |
| | (SEQ ID NO: 99) | (SEQ ID NO: 113) | (SEQ ID NO: 125) |
| BRCA1 | GGTTAGGATTTTTCTCAT | ATTGTGGATATTTAATTCGAGT | GAGCAGAGGGTGAAGGCCTCC |
| | TCTGAATAGAATCA | TCCATATTGC | TGAGCGCAGGG |
| | (SEQ ID NO: 100) | (SEQ ID NO: 114) | (SEQ ID NO: 126) |
| TARDBP | CTTGTGTTTCATATTCCG | GTCCATCTATCATATGTCGCTG | CTTTGAATGACCAGTCTTAAG |
| | TAAAACGAACAAAG | TGACATTACT | ATCTTTCTTGA |
| | (SEQ ID NO: 101) | (SEQ ID NO: 115) | (SEQ ID NO: 127) |
| SMARCA | GTCCATCCAGTCGACAA | CCGACAAAACAAATGACACGG | AAAAGTTGAGTAAGGCCCACA |
| 1 | TACTCATAACCACGC | AGAGATGGGAC | GTTCATGCAGG |
| | (SEQ ID NO: 102) | (SEQ ID NO: 116) | (SEQ ID NO: 128) |
| CKB | GCTTGTCGAAGAGGAAG | TTGATATGCACACCTGCCCGC | CGAGAACTTCTCATGCTTGCCC |
| | TGGTCGTCGATGAGC | AGCCCGGTGCC | AGGTTGGGCA |
| | (SEQ ID NO: 103) | (SEQ ID NO: 117) | (SEQ ID NO: 129) |
| ENO1 | CATCCATCTCGATCATCA | TTCCCGCGAGAGTCAAAGATC | GACCCCCTTCTCAACGGCACC |
| | GTTTGTCAATCTTC | TCCCTGGCATG | AGCTTTGCAGA |
| | (SEQ ID NO: 104) | (SEQ ID NO: 118) | (SEQ ID NO: 130) |
| MECP2 | CAGAGTGGTGGGCTGAT | GGCAGAAGCTTCCGGCACAGC | CAAATACACATCATACTTCCC |
| | GGCTGCACGGGCTCA | CGGGGCGGAGC | AGCAGAGCGGC |
| | (SEQ ID NO: 105) | (SEQ ID NO: 119) | (SEQ ID NO: 131) |
| UBE3A | CTCGAGAGTATACATTGT | GAGATTTCTATTCTCCATTACG | AAATTCCACATACAACTGCTT |
| | GATACGTCAAGTCA | ATAATGAACA | CTTCAAGTCTG |
| | (SEQ ID NO: 106) | (SEQ ID NO: 120) | (SEQ ID NO: 132) |
| SMAD4 | TCCACCTTGTCTATGGCA | AGCTTCTTTACCAAACTTTCAA | TCCAATGTTCTCTGTATGGTAA |
| | CATCAAACTATGCA | TTGCTCTTTT | CACATTTACT |
| | (SEQ ID NO: 107) | (SEQ ID NO: 121) | (SEQ ID NO: 133) |
| GFP | CAGCTTGCCGGTGGTGC | TGAAGCACTGCACGCCGTAGG | AGGATGTTGCCGTCCTCCTTGA |
| | AGATGAACTTCAGGG | TCAGGGTGGTC | AGTCGATGCC |
| | (SEQ ID NO: 108) | (SEQ ID NO: 122) | (SEQ ID NO: 134) |
| RFP | CTTGAAGCCCTCGGGGA | | |
| | AGGACAGCTTCAAGT | | |
| | (SEQ ID NO: 109) | | |
| NT | TCTCCGAACGTGTCACGT | | |
| | CTTTAGCGACTAAA | | |
| | (SEQ ID NO: 110) | | |

TABLE 4
Sequences used

| Target | Csm crRNA intronic |
|---|---|
| XIST | GTCAGTAAATGAACCTTTCCTATCCCACGTGT (SEQ ID NO: 135) |
| BRCA1 | CCAGTCATGATCATTCCTGATCACATATTAAG (SEQ ID NO: 136) |
| Target | Cas13 crRNA |
| XIST | GCCACTTGAACACTGCGACAGAACTGGATC (SEQ ID NO: 137) |
| MALAT1 | GCCGCCTGCTACCTTCATCACCAAATTGCA (SEQ ID NO: 138) |
| CKB | GCTTGTCGAAGAGGAAGTGGTCGTCGATGA (SEQ ID NO: 139) |
| SMAD4 | TCCACCTTGTCTATGGCACATCAAACTATG (SEQ ID NO: 140) |
| NT | TCTCCGAACGTGTCACGTCTTTAGCGACTA (SEQ ID NO: 141) |
| Target | shRNA |
| XIST | GTTCTGTCGCAGTGTTCAAGTcctgacccaACTTGAACACTGCGACAGAAC (SEQ ID NO: 142) |
| MALATI | GTGATGAAGGTAGCAGGCGGCcctgacccaGCCGCCTGCTACCTTCATCAC (SEQ ID NO: 143) |
| CKB | GACCACTTCCTCTTCGACAAGcctgacccaCTTGTCGAAGAGGAAGTGGTC (SEQ ID NO: 144) |
| SMAD4 | GATGTGCCATAGACAAGGTGGcctgacccaCCACCTTGTCTATGGCACATC (SEQ ID NO: 145) |
| NT | GCTAAAGACGTGACACGTTCGcctgacccaCGAACGTGTCACGTCTTTAGC (SEQ ID NO: 146) |
| Spacer | |
| length | Csm crRNA (GFP) |
| 24 nt | CAGCTTGCCGGTGGTGCAGATGAA (SEQ ID NO: 147) |
| 28 nt | CAGCTTGCCGGTGGTGCAGATGAACTTC (SEQ ID NO: 148) |
| 32 nt | CAGCTTGCCGGTGGTGCAGATGAACTTCAGGG (SEQ ID NO: 149) |
| 36 nt | CAGCTTGCCGGTGGTGCAGATGAACTTCAGGGTCAG (SEQ ID NO: 150) |
| 40 nt | CAGCTTGCCGGTGGTGCAGATGAACTTCAGGGTCAGCTTG (SEQ ID NO: 151) |
| 44 nt | CAGCTTGCCGGTGGTGCAGATGAACTTCAGGGTCAGCTTGCCGT (SEQ ID NO: 152) |
| 48 nt | CAGCTTGCCGGTGGTGCAGATGAACTTCAGGGTCAGCTTGCCGTAGGT (SEQ ID NO: 153) |
| Target | Live-cell imaging Csm crRNA |
| XIST | AAAAGCAGGTATCCGCGGCCCCGATGGGCAAA (SEQ ID NO: 154) |
| MALAT1 | AGCTTCCTTCACCAAATCGCACTGGCTCCTGG (SEQ ID NO: 155) |
| NEAT1 | CACCATTACCAACAATACCGACTCCAACAGCC (SEQ ID NO: 156) |
| Target | RNA FISH probe |
| XIST | /5Cy3/GGGCACTCCCTGCTGGAAGGGAA (SEQ ID NO: 157) |
| | /5Cy3/AATTGTGCACCTTGACTGTCCAAA (SEQ ID NO: 158) |
| | /5Cy3/TCTGAGAGTAGGACCTTATTCA (SEQ ID NO: 159) |
| | /5Cy3/TCAGCACCCCTGCTGTACTGCAAA (SEQ ID NO: 160) |
| MALAT1 | SMF-2035-1 (LGC Biosearch Technologies) |
| NEAT1 | SMF-2036-1 (LGC Biosearch Technologies) |

TABLE 5
Plasmid sequences used

| Plasmid | pDAC338 |
|---|---|
| Description | Expression of Csm1 |
| Features | Pcmv-FLAG-NLS-Csm1-pA |
| SEQ ID NO: | 161 |
| Plasmid | pDAC803 |
| Description | Expression of Csm1(DNase mut) |
| Features | Pcmv-FLAG-NLS-Csm1(DNase mut)-pA |
| SEQ ID NO: | 162 |
| Plasmid | pDAC804 |
| Description | Expression of Csm1(cA mut) |
| Features | Pcmv-FLAG-NLS-Csm1(cA mut)-pA |
| SEQ ID NO: | 163 |
| Plasmid | pDAC309 |
| Description | Expression of Csm2 |
| Features | Pcmv-FLAG-NLS-Csm2-pA |
| SEQ ID NO: | 164 |
| Plasmid | pDAC310 |
| Description | Expression of Csm3 |
| Features | Pcmv-FLAG-NLS-Csm3-pA |
| SEQ ID NO: | 165 |
| Plasmid | pDAC327 |
| Description | Expression of Csm3(RNase mut) |
| Features | Pcmv-FLAG-NLS-Csm3(RNase mut)-pA |
| SEQ ID NO: | 166 |
| Plasmid | pDAC339 |
| Description | Expression of Csm4 |
| Features | Pcmv-FLAG-NLS-Csm4-pA |
| SEQ ID NO: | 167 |
| Plasmid | pDAC312 |
| Description | Expression of Csm5 |
| Features | Pcmv-FLAG-NLS-Csm5-pA |
| SEQ ID NO: | 168 |
| Plasmid | pDAC307 |
| Description | Expression of Cas6 |
| Features | Pcmv-FLAG-NLS-Cas6-pA |
| SEQ ID NO: | 169 |
| Plasmid | pDAC324 |
| Description | Expression of crRNA |
| Features | Pu6-crRNA-pT |
| SEQ ID NO: | 170 |
| Plasmid | pDAC439 |
| Description | Expression of Csm complex from single promoter; RFP |
| | backbone |
| Utility | RNA KD |
| Features | Pcmv-FLAG-NLS-Csm5-2A-FLAG-NLS-Csm4-2A- |
| | FLAG-NLS-Csm3-2A-FLAG-NLS-Csm2-pA; Pcmv- |
| | RFP-2A-FLAG-NLS-Cas6-2A-FLAG-NLS- |
| | Csm1(Dnase/cA mut)-pA; Pu6-crRNA-pT |
| SEQ ID NO: | 171 |
| Plasmid | pDAC435 |
| Description | Expression of Csm complex from separate promoters; |
| | RFP backbone |
| Utility | RNA KD |
| Features | Pcmv-FLAG-NLS-Csm1-pA; Pcmv-FLAG-NLS-Csm2- |
| | pA, Pcmv-FLAG-NLS-Csm3-pA; Pcmv-FLAG-NLS- |
| | NLS-Cas6-pA; Pu6-crRNA-pT; Pcmv-RFP-pA |
| | Csm4-pA; Pcmv-FLAG-NLS-Csm5-pA; Pcmv-FLAG- |
| | NLS-Cas6-pA; Pu6-crRNA-pT; Pcmv-RFP-pA |
| SEQ ID NO: | 172 |
| Plasmid | pDAC446 |
| Description | Expression of Csm complex from separate promoters; |
| | GFP backbone |
| Utility | RNA KD |
| Features | Pcmv-FLAG-NLS-Csm1-pA; Pcmv-FLAG-NLS-Csm2- |
| | pA, Pcmv-FLAG-NLS-Csm3-pA; Pcmv-FLAG-NLS- |
| | Csm4-pA; Pcmv-FLAG-NLS-Csm5-pA; Pcmv-FLAG- |
| | NLS-Cas6-pA; Pu6-crRNA-pT; Pcmv-GFP-pA |
| SEQ ID NO: | 173 |
| Plasmid | pDAC627 |
| Description | Expression of Csm complex from separate promoters; |
| | Puro backbone |
| Utility | RNA KD |
| Features | Pcmv-FLAG-NLS-Csm1-pA; Pcmv-FLAG-NLS-Csm2- |
| | pA, Pcmv-FLAG-NLS-Csm3-pA; Pcmv-FLAG-NLS- |
| | Csm4-pA; Pcmv-FLAG-NLS-Csm5-pA; Pcmv-FLAG- |
| | NLS-Cas6-pA; Pu6-crRNA-pT; Pcmv-Puro-pA |
| SEQ ID NO: | 174 |
| Plasmid | pDAC569 |
| Description | Expression of Csm complex (RNase mut) from separate |
| | promoters; GFP backbone |
| Utility | RNA binding/tethering/pulldown |
| Features | Pcmv-FLAG-NLS-Csm1-pA; Pcmv-FLAG-NLS-Csm2- |
| | pA, Pcmv-FLAG-NLS-Csm3(RNase mut)-pA; Pcmv- |
| | FLAG-NLS-Csm4-pA; Pcmv-FLAG-NLS-Csm5-pA; |
| | Pcmv-FLAG-NLS-Cas6-pA; Pu6-crRNA-pT; Pcmv- |
| | GFP-pA |
| SEQ ID NO: | 175 |
| Plasmid | pDAC565 |
| Description | Expression of Csm-GFP complex (RNase mut) from |
| | separate promoters |
| Utility | RNA imaging |
| Features | Pcmv-FLAG-NLS-Csm1-pA; Pcmv-FLAG-NLS-Csm2- |
| | pA, Pcmv-FLAG-NLS-Csm3(RNase mut)-GFP-pA; |
| | Pcmv-FLAG-NLS-Csm4-pA; Pcmv-FLAG-NLS-Csm5- |
| | pA; Pcmv-FLAG-NLS-Cas6-pA; Pu6-crRNA-pT |
| SEQ ID NO: | 176 |
| Plasmid | pDAC689 |
| Description | Expression of Cas13; RFP backbone |
| Utility | RNA KD |
| Features | Pcmv-NLS-Cas13-NLS-pA; Pu6-crRNA-pT; Pcmv-RFP- |
| | pA |
| SEQ ID NO: | 177 |
| Plasmid | pDAC690 |
| Description | Expression of shRNA; RFP backbone |
| Utility | RNA KD |
| Features | Pu6-shRNA-pT; Pcmv-RFP-pA |
| SEQ ID NO: | 178 |

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method for modifying a target RNA in a eukaryotic cell, the method comprising introducing into the eukaryotic cell:
a) one or more nucleic acids comprising nucleotide sequences encoding a multi-subunit Type III CRISPR-Cas effector polypeptide, wherein the multi-subunit Type III CRISPR-Cas effector polypeptide comprises at least 5 subunits; and
b) one or more guide RNAs, wherein each of the one or more guide RNAs comprises: i) a targeting region that comprises a nucleotide sequence that is complementary to a target sequence in the target RNA; and ii) a protein-binding region that binds to the multi-subunit Type III CRISPR-Cas effector polypeptide; or a nucleic acid comprising a nucleotide sequence encoding the guide RNA, wherein the multi-subunit Type III CRISPR-Cas effector polypeptide is produced in the cell and forms a complex with the guide RNA, and wherein the complex binds to the target RNA and results in modification of the target RNA in the cell.

2. The method of claim 1, wherein the one or more nucleic acids comprises one or more recombinant expression vectors selected from a recombinant adeno-associated virus vector, a recombinant lentivirus vector, a recombinant adenovirus vector, and a recombinant retroviral vector.

3. (canceled)

4. The method of claim 1, wherein the nucleotide sequences encoding the at least 5 subunits are operably linked to a single promoter.

5. The method of claim 1, wherein the nucleotide sequences encoding the at least 5 subunits are operably linked to two or more different promoters.

6-7. (canceled)

8. The method of claim 1, wherein the one or more nucleic acids comprising nucleotide sequences encoding the multi-subunit Type III CRISPR-Cas effector polypeptide comprise a nucleotide sequence encoding the one or more guide RNAs.

9-10. (canceled)

11. The method of claim 1, wherein the target RNA is a coding RNA.

12-15. (canceled)

16. The method of claim 1, wherein the target RNA is an endogenous RNA or a viral RNA.

17-19. (canceled)

20. The method of claim 1, wherein the modifying comprises cleavage of the target RNA.

21. The method of claim 1, wherein the modifying comprises methylation or adenylation.

22. (canceled)

23. The method of claim 1, wherein the eukaryotic cell is in vitro.

24. (canceled)

25. The method of claim 1, wherein the multi-subunit Type III CRISPR-Cas effector polypeptide is a Type IIIA CRISPR-Cas effector polypeptide comprising Cas10/Csm1, Csm2, Csm3, Csm4, and Csm5 polypeptides; or is a Type IIIB CRISPR-Cas effector polypeptide comprising Cmr1, Cmr2, Cmr3, Cmr4, Cmr5, and Cmr6 subunits.

26. The method of claim 25, wherein the Cas10/Csm1, Csm2, Csm3, Csm4, and Csm5 polypeptides each independently comprise an amino acid sequence having at least 50% amino acid sequence identity to any of the amino acid sequences of the Cas10/Csm1, Csm2, Csm3, Csm4, and Csm5 polypeptides of SEQ ID Nos: 1-5 or FIG. 7A-7E; and wherein the Cmr1, Cmr2, Cmr3, Cmr4, Cmr5, and Cmr6 polypeptides each independently comprise an amino acid sequence having at least 50% amino acid sequence identity to any of the amino acid sequences of the Cmr1, Cmr2, Cmr3, Cmr4, Cmr5, and Cmr6 polypeptides depicted in FIG. 6A-6F.

27-28. (canceled)

29. The method of claim 1, wherein the multi-subunit Type III CRISPR-Cas effector polypeptide comprises one or more amino acid substitutions that reduce DNAse activity.

30. (canceled)

31. The method of claim 1, wherein the multi-subunit Type III CRISPR-Cas effector polypeptide comprises one or more amino acid substitutions that reduce polymerization of ATP into a cyclic oligoadenylate (cA) molecule, wherein the one or more amino acid substitutions that reduce polymerization of ATP to cA comprise a substitution of D577, a substitution of D578, or a substitution of both D577 and D578 of a Csm10/Csm1 polypeptide.

32. (canceled)

33. A method of detecting a target RNA in a eukaryotic cell, the method comprising contacting the target RNA with a complex comprising:
a) a Type III CRISPR-Cas effector polypeptide, wherein the Type III CRISPR-Cas effector polypeptide comprises 5 subunits, wherein the Type II CRISPR-Cas effector polypeptide does not substantially cleave the target RNA; and
b) a guide RNA that comprises: i) a targeting region that comprises a nucleotide sequence that is complementary to a target sequence in the target RNA; and ii) a protein-binding region that binds to the Type III CRISPR-Cas effector polypeptide.

34. The method of claim 33, wherein one or more of the subunits comprises a detectable label.

35-46. (canceled)

47. A composition useful for modifying a target RNA in a eukaryotic cell, the composition comprising:
a) one or more nucleic acids comprising nucleotide sequences encoding a multi-subunit Type III CRISPR-Cas effector polypeptide, wherein the multi-subunit Type III CRISPR-Cas effector polypeptide comprises at least 5 subunits; and
b) one or more guide RNAs, wherein each of the one or more guide RNAs comprises: i) a targeting region that comprises a nucleotide sequence that is complementary to a target sequence in the target RNA; and ii) a protein-binding region that binds to the multi-subunit Type III CRISPR-Cas effector polypeptide; or a nucleic acid comprising a nucleotide sequence encoding the guide RNA, wherein, when the eukaryotic cell is contacted with the composition, the multi-subunit Type III CRISPR-Cas effector polypeptide is produced in the cell and forms a complex with the guide RNA, and wherein the complex binds to the target RNA and results in modification of the target RNA in the cell.

48. The composition of claim 47, wherein the one or more nucleic acids comprises one or more recombinant expression vectors selected from a recombinant adeno-associated virus vector, a recombinant lentivirus vector, a recombinant adenovirus vector, and a recombinant retroviral vector.

49. (canceled)

50. The composition of claim 47, wherein the nucleotide sequences encoding the at least 5 subunits are operably linked to one, two or more promoters, and wherein the promoters are constitutive or regulatable promoters in any combination.

51-52. (canceled)

53. The composition of claim 47, wherein the target RNA is a coding RNA.

54-63. (canceled)

64. The composition of claim 47, wherein the multi-subunit Type III CRISPR-Cas effector polypeptide is a Type IIIA CRISPR-Cas effector polypeptide comprising Cas10/Csm1, Csm2, Csm3, Csm4, and Csm5 polypeptides; or is a Type IIIB CRISPR-Cas effector polypeptide comprising Cmr1, Cmr2, Cmr3, Cmr4, Cmr5, and Cmr6 subunits.

65. The composition of claim 64, wherein the Cas10/Csm1, Csm2, Csm3, Csm4, and Csm5 polypeptides each independently comprise an amino acid sequence having at least 50% amino acid sequence identity to the amino acid sequences of the Cas10/Csm1, Csm2, Csm3, Csm4, and Csm5 polypeptides depicted in FIG. 5; and wherein the Cmr1, Cmr2, Cmr3, Cmr4, Cmr5, and Cmr6 polypeptides each independently comprise an amino acid sequence having at least 50% amino acid sequence identity to the amino acid sequences of the Cmr1, Cmr2, Cmr3, Cmr4, Cmr5, and Cmr6 polypeptides depicted in FIG. 6.

66-71. (canceled)


SEQUENCE LISTING (180 sequences)

SEQ ID NO: 1 - protein, 758 aa, Streptococcus thermophilus
MKKEKIDLFYGALLHDIGKVIQRATGERKKHALVGADWFDEIADNQVISDQIRYHMANYQSDKLGNDHLAYITYIADNIASGVDRRQSNEESDEDTSAKIWDTYTNQADIFNVFGAQTDKRYFKPTVLNLKSKPNFASATYEPFSKGDYAAIATRIKNELAEFEFNQVQIDSLLNLFEATLSFVPSSTNTKEIADISLADHSRLTAAFALAIYDYLEDKGRHNYKEDLFTKVSAFYEEEAFLLASFDLSGIQDFIYNINIATNGAAKQLKARSLYLDFMSEYIADSLLDKLGLNRANMLYVGGGHAYFVLANTEKTVETLVQFEKDFNQFLLANFQTRLYVAFGWGSFAAKDIMSELNSPESYRQVYQKASRMISKKKISRYDYQTLMLLNRGGKSSERECEICHSVENLVSYHDQKVCDICRGLYQFSKEIAHDHFIITENEGLPIGPNACLKGVAFEKLSQEAFSRVYVKNDYKAGTVKATHVFVGDYQCDEIYNYAALSKNENGLGIKRLAVVRLDVDDLGAAFMAGFSQQGNGQYSTLSRSATFSRSMSLFFKVYINQFASDKKLSIIYAGGDDVFAIGSWQDIIAFTVELRENFIKWTNGKLTLSAGIGLFADKTPISLMAHQTGELEEAAKGNEKDSISLFSSDYTFKFDRFITNVYDDKLEQIRYFFNHQDERGKNFIYKLIELLRNHDRMNMARLAYYLTRLEELTRETDRDKFKTFKNLFYSWYTNKNDKDRKEAELALLLYIYEIRKD

SEQ ID NO: 2 - protein, 756 aa, Streptococcus salivarius
MKKEKIDLFYGALLHDIGKVIQRATGERKKHALVGADWFDEIADNQVISDQIRYHMANYQSDKLGNDHLAYITYIADNIASGVDRRQSNEESDEDASAKIWDTYTNQADIFNVFGAQTDKRYFKPTVLNLKSEPNFASATYEPFSKGDYAAIATRIKNELAEFEFNQAQIDSLLNLFEATLSFVPSSTNTKEIADISLAEHSRLTAAFALAIYDYLEDKGRHNYKEDLFTKASAFYEEEAFLLASFDLSGIQDFIYNIATSGAAKQLKARSLYLDFMSEYIADSLLDKLGLNRANLLYVGGGHAYFVLANTEKTVETLVQFEKDFNQFLLANFQTRLYVAFGWGSFAAKDIMSELNSPESYRQVYQKASRMISEKKISRYDYQTLMLLNRGGKSSERECEICHSVENLVSYHDQKVCDICRGLYQFSKEIAHDHFIITENEGLPIGPNACLKGVAFEKLSHEEFSRVYVKNDYKAGTVKATHVFVGDYQCDEIYNYAALSKNENGLGIKRLAVVRLDVDDLGAAFMAGFSQQGNGQYSTLSRSATFSRSMSLFFKVYINQFASDKKLSIIYAGGDDVFAIGSWQDIISFTVELRQNFIKWTNGKLTLSAGIGLFADKTPISLMAHQTGELEEAAKGNEKDSISLFSSDYTFKFDQFITNVYDDKLEQIRYFFNHQDERGKNFIYKLIELLRNHDRMNMARLAYYLTRLEELTRETDRDKFKTFKSLFYSWYTNKDDKDRKEAELALLLYVYEIRKD

SEQ ID NO: 3 - protein, 493 aa, Streptococcus vestibularis
MKKEKIDLFYGALLHDIGKVIQRATGERKKHALVGADWFDEIADNQVISDQIRYHMANYQSDELGNDHLAYITYIADNIASGIDRRQSNEESDEEASAKIWDTYINQADIFNVFGAKTDKRYFKPTVLNLKSEPNFASATYEPFSKGDYAAIATRIKNELAKFEFNQAQIDSLLNLFEATLSFVPSSTNTKEIADISLAEHSRLTAAFALAIYDYLEDKDRHNYKEDLFTKASAFYEEEAFLLASFDLSGIQDFIYNIATSGAAKQLKSRSLYLDFMSEYIADSLLDKLGLNRANLLYVGGGHAYFVLANTEKTVETLVQFEKDFNQFLLANFQTRLYVAFGWGSFAAKDIMSELNSPESYRQVYQKASRMISEKKISRYDYQTLILLNRGGKSSERECEICHSVENLVSYHDQKVCDICRGLYQFSKEIAHDHFIITKSEGLPIGPNACLKGVAFEKLSQEDFSRVYVKNDYKAGTVKATHVFVGDYLCDEI

SEQ ID NO: 4 - protein, 769 aa, Streptococcus sobrinus
MNSDKINLFYGGLFHDIGKVVQRATGERKKHALIGADWFERFSDNKSISQQIRYHMASYQTNLDNDNLAYITYIADNVASGVDRRTSQEEGDQSANQKIWDTYTNQEDIFNVFGKNRSQRYFEPRELTVTTVPNFAEQSHRKFSKGDYAAILHHIEESVNLINFDESYINSALNLLEATLSFVPASTNTKEIADISLYEHSKLTAGFAAAIYDYLRDSNRTDFKKELFKNTQNFYKEQAFLLASFDLSGIQDFIYNIATKGAAKQLKARSLYLDFMSEHIVDSLLEKLELTRANLMYVGGGHAYFVFPNTPKTKHTLSDFEKSFNQFLLEQFQTRLYVAFGWSEFSASDVMSCSNSPETYRAIYQRASRKISDKKLSRYDWQTLLKLNQKGKREGRECQICHSVDGLTVYHEQTLCHICNHLRQFAKMTNSTHFLVSSNNAGLPIGPTAYLSACDEKQIKQGIEGRIYTKNDFQTGSGLSTHVFIGDYTAGSIDEYAGLSTEHLQPNGEKAGIRRLAVVRLDVDDLGAGFMAGFSYQNAGQYNTFSRTATFSRSMTQFFKVYINQFAQGKKISIIYSGGDDVFAIGTWQDIITFTIELRQNFIRWTNRKLTLSAGIGLFADKTPVSIMAGLTGELEEAAKGNKNHNKDSLALFDQHFVFDFDSFISEIYESKLPLIRSYFNQQDERGKSFAHRLLELIRSQEKIDIARLAYYLSRMEDAASQKDAFKTFKTKFFKWATSGEEAKKQVEAALVLYIYESRKDTRYDNFDR

SEQ ID NO: 5 - protein, 759 aa, Streptococcus suis
MKQEKSDIFYGALLHDIGKVVQRATGERAKHALVGTDWLKNFTDNATILAHVKNHMASHQDSLPEDHTAYITYIADNIASGVDRRKSCEETEIAGQIWDHYTNQADIFNVFGAEPSQRYFQPVELDVQKKPNLASRENSHFTKGEYSRILQRITEAFRVMDYTPAYHSSFLNLLEATLSYVPSSTNTKEIADISLFEHSKLTAGFALAIYDYLKDQGRTQFSKELFRGATDFYKEEAFLLASFDLSGIQDFIYNIATSGAAKQLKARSFYLDFMGEHLVDSLLDRLELNRANLLYIGGGHAYLLLANTASTRQILEEFEKEMNAFLLANVQTRLYVAFGWSAFAAQDIMVDSQSAETYRQIYQKVSQQISAKKLRRYNWKTLMVLNRGGKKAGRECVICHAVDSLEEKHGKLLCGLCSGLHDFSKKIQFKHYLVSGHPSSLPVGPDAYLHALDSDGVKKKPEGRIYSKNDFFTGEQQATHIYVGDYQAAEIYDYAQLSTEVINSRGERSGIKRLAVVRLDVDDLGAAFMAGYSVQGNGSYNTFSRTASFSRSMSQFFKVYINQFASGKAISIIYSGGDDVFAIGTWQDIIAFTIDLRQQFINWTNGKLTLSAGIGLFTDKTPISLMARETGYLEEAAKANEKDSISLFEEEFTFKFDDFIQRVYGEYLTKVRTFFNTMDDYGKSFLYRLIELLRSTDRLDIARAAYYLTRLEEKAKGENKELFKEFKVFFMNVAMSNQAAKKELEMALTLYVYEIRKDS

SEQ ID NO: 6 - protein, 771 aa, Streptococcus sobrinus
MNSDKINLFYGGLFHDIGKVVQRATGERKKHALIGADWFERFSDNKSISQQIRYHMASYQTNLDNDNLAYITYIADNVASGVDRRTSQEEGDQSANQKIWDTYTNQEDIFNVFGKNRSQRYFEPRELTVTTVPNFAEQSHRKFSKGDYAAILHHIEESVNLINFDESYINSALNLLEATLSFVPASTNTKEIADISLYEHSKLTAGFAAAIYDYLRDSNRTDFKKELFKNTQNFYKEQAFLLASFDLSGIQDFIYNIATKGAAKQLKARSLYLDFMSEHIVDSLLEKLELTRANLMYVGGGHAYFVFPNTPKTKHTLSDFEKSFNQFLLEQFQTRLYVAFGWSEFSASDVMSCSNSPETYRAIYQRASRKISDKKLSRYDWQTLLKLNQKGKREGRECQICHSVDGLTVYHEQTLCHICNHLRQFAKMTNSTHFLVSSNNAGLPIGPTAYLSACDEKQIKQGIEGRIYTKNDFQTGSGLSTHVFIGDYTAGSIDEYAGLSTEHLQPNGEKAGIRRLAVVRLDVDDLGAGFMAGFSYQNAGQYNTFSRTATFSRSMTQFFKVYINQFAQGKKISIIYSGGDDVFAIGTWQDIITFTIELRQNFIRWTNRKLTLSAGIGLFADKTPVSIMAGLTGELEEAAKGNKNHNKDSLALFDQHFVFDFDSFISEIYESKLPLIRSYFNQQDERGKSFAHRLLELIRSQEKIDIARLAYYLSRMEDAAKDSQKDAFKTFKTKFFKWATSGEEAKKQVEAALVLYIYESRKDTRYDNFDR

SEQ ID NO: 7 - protein, 126 aa, Streptococcus thermophilus
MTILTDENYVDIAEKAILKLERNTRNRKNPDAFFLTTSKLRNLLSLTSTLFDESKVKEYDALLDRIAYLRVQFVYQAGREIAVKDLIEKAQILEALKEIKDRETLQRFCRYMEALVAYFKFYGGKD

SEQ ID NO: 8 - protein, 126 aa, Streptococcus salivarius
MTILTDENYVDIAEKAILKLERNTRNRKNPDAFFLTTSKLRNLLSLTSTLFDESKVKEYDALLDRIAYLRVQFVYQAGREVAVKDLVEKAQILEALKEIKDRETLQRFCRYMEALVAYFKFYGGKD

SEQ ID NO: 9 - protein, 105 aa, Faecalibacterium prausnitzii
NTRNRKNPDAFFLTTSKLRNLLSLTSTLFDESKVKEYDDLFDRIAYLRVQFVYQAGREIAVKDLIEKAQILEALKEIKDRETLQRFCRYMEALVAYFKFYGGKDK

SEQ ID NO: 10 - protein, 126 aa, Streptococcus mitis
MATLTDDNYVDKAENVIKSLNRNTRDFRNPDAFLLTTSKIRNLLSLTSTLFDESKVRDYEALADKIAYLRVQFVYQSGRETAVKDLVKKAEILDILKEINNKESLQRFCRYMEALVAYFKFYGGKD

SEQ ID NO: 11 - protein, 126 aa, Streptococcus oralis
MAILTDDNYVDKAENVIKSLSRNTRDSRNPEAFLLTTSKIRNLLSLTSTLFDESKVREYKDLADKIAYLRVQFVYQSGRETAVKDLVKKAEILDILKEINNKESLQRFCRYMEALVAYFKFYGGKD

SEQ ID NO: 12 - protein, 126 aa, Streptococcus downii
MEILRDDNYVDKAENVIKSLNRNTRDSRNPEAFLLTTSKIRNLLSLTSTLFDESKVREYKDLADKIAYLRVQFVYQSGRETAVKDLVKKAEILDILKEINNKESLQRFCRYMEALVAYFKFYGGKD

SEQ ID NO: 13 - protein, 126 aa, Fusobacterium periodonticum
MAILTDDNYVDKAEKTIKNLVTDKRNFKNRNSDVLSMSKLRNLLSLTSTLFDESKVREYEELKDRIAYLRVQFVYQSGREEAVLDLVQKGEILPILKEINSRESLQRFCRYMEALVAYFKFYGGND

SEQ ID NO: 14 - protein, 124 aa, Weissella soli
MAILTDETYVDKAEKVIKSLDRDNRNPAAFVLTTTKIRNLLSLTSNLYDESLGKQSLDGLTDKLAYLRVQFVYQAGREKSVKQLVENAEILGALKEINDVASLQRFSRYMEALVAYFKFEGGKD

SEQ ID NO: 15 - protein, 148 aa, Ligilactobacillus sp. AF129
MINNQSRNNNRYQKPAFKKPQLNISFTEESYVSEAEKVILSLKENNFKHGRDVLTTSQIRNLLSLTSSILNDLVFKEFEQVMDKLSYLRIQFVYQSGRNGAVKDLVETAQILKILENVQAKKDKETLIRFCHYMEALVAYFKYYGGKD

SEQ ID NO: 16 - protein, 220 aa, Streptococcus thermophilus
MTFAKIKFSAQIRLETGLHIGGSDAFAAIGAIDSPVIKDPITNLPIIPGSSLKGKMRTLLAKVYNEKVAEKPSDDSDILSRLFGNSKDKRFKMGRLIFRDAFLSNADELDSLGVRSYTEVKFENTIDRITAEANPRQIERAIRTSTFDFELIYEITDENENQVEEDFKVIRDGLKLLELDYLGGSGSRGYGKVAFENLKATTVFGNYDVKTLNELLTAEV

SEQ ID NO: 17 - protein, 220 aa, Streptococcus salivarius
MTFAKIKFSAQIRLETGLHIGGSDAFAAIGAIDSPVIKDPITNLPIIPGSSLKGKMRTLLAKVYNEKVAEKPSDDTDILSRLFGNSKDKRFKMGRLIFRDAFLSNADELDSLGVRSYTEVKFENTIDRITAEANPRQIERAIRNSTFDFELIYEITDENENQVEEDFKVIRDGLKLLELDYLGGSGSRGYGKVAFENLKATTVFGNYDVKTLNELLTAEV

SEQ ID NO: 18 - protein, 220 aa, Streptococcus equinus
MTFAKIKFSAQIRLETGLHIGGSDAFAAIGAIDSPVIKDPITNLPIIPGSSLKGKMRTLLAKVYNEKVAEKPSDDTDILSRLFGNSKDKRFKMGRLIFRDAFLSNADELDSLGVRSYTEVKFENTIDRITAEANPRQIERAIRNSTFDFELIYEITDKNENQVEEDFKVIRDGLKLLELDYLGGSGSRGYGKVAFENLKATTVFGNYDVKTLNELLTAEV

SEQ ID NO: 19 - protein, 220 aa, Faecalibacterium prausnitzii
MTFAKIKVSAQIRLETGLHIGGSDAFAAIGAIDSPVIKDPITNIPIIPGSSLKGKMRTLLAKVYNEKVAEKPSDDSDILSRLFGNSKDKRFKMGRLIFRDAFLSNADELDSLGVRSYTEVKFENTIDRITAEANPRQIERAIRNSTFDFELIYEITDENENQVEEDFKVIRDGLKLLELDYLGGSGSRGYGKVAFEKLKATNVFGNYDVKTLNELLTAEV

SEQ ID NO: 20 - protein, 220 aa, Streptococcus vestibularis
MTFAKIKFSAQIRLETGLHIGASNAFAAIGAIDSPVIKDPITNLPIIPGSSLKGKMRTLLAKVYNEEIVKNPSQDSDVLSRLFGNSEDKRFKMGRLIFRDAFLSNADELDSLGARSYTEVKFENTIDRITAEATPRQIERAIRNSTFDFELIYEITDENENQVEEDFKVIRDGLKLLELDYLGGSGSRGYGKVAFENLKATTVFGNYDVKTLNELLTAEV

SEQ ID NO: 21 - protein, 220 aa, Streptococcus oralis
MAFAKIKVTAQIRLETGLHIGTSNAFAAIGATDSPVIKDPITNLPIIPGSSIKGKMRTLLSKVHNERLAKKPGEDGEILSRLFGNSSDNRYKMGRLIFRDAFLINKDKLDSLGVKSYTEVKFENTIDRITAEANPRQIERAIRDSIFGFELIYEVTDKSQAQVEEDFKVILDGLKLLELDYLGGSGSRGYGKVVFENLKANTVFGNYDVSRLNELLTTEV

SEQ ID NO: 22 - protein, 220 aa, Fusobacterium periodonticum
MAFAKIQVTGQIRLETGLHIGTSNAFAAIGATDSPVIKDPITNLPIIPGSSIKGKMRTLLAKVYNTTLAEKPGDDGEIISRLFGNSSDKDYKVGRLIFRDAFLTNKKDLDSRAVKGYTEVKFENTIDRITAEANPRQIERAIRDSIFGFELIYEVTDKSQAQVEEDFKVILDGLKLLELDYLGGSGSRGYGKVVFEDLKANTVFGNYDVSRLNELLTTEV

SEQ ID NO: 23 - protein, 221 aa, Limosilactobacillus mucosae
MMLSKIKISGTIELESGLHIGGSDAFAAIGAIDSPVIKDPLTNLPIIPGSSLKGKMRSLLAKAYNETIAQTPNDDDERITRLFGASDGGGENGDQPVPARLIFRDSFLNNAQELLDKGAEGYTEAKFENTIDRTTAIANPRQIERAIRGSEFNFELIYDLTDEQEIDEDMQTLLDGFKLLELDYLGGSGSRGYGKIKFINLHAETVFGDYDASAINEELGA

SEQ ID NO: 24 - protein, 222 aa, Weissella soli
MAYSKVKITGLIELETGLHIGGSSAFAAIGATDSPVIKDPLTNLPIIPGSTLKGKVRSLLARAFNERTAEKPHQDTESIRRLFGDSESDEYKIGRLIFRDAHLTNADELTNKGARTLTEVKFENTIDRITAMANPRQIERAIRGSQFKFELIYDVQEETDVPEDIDLIVSGLQLLELDYLGGSGSRGYGKVDFKNFIVETAFGEFDSTELQKKLDELTPAEV

SEQ ID NO: 25 - protein, 222 aa, Ligilactobacillus ruminis
MTKLSKIRFSGVIELESGLHIGGGDAFAAIGAIDSPIIKDSVTNLPIIPGSSLKGKMRTLLARSYNEEVAKTPNEDNERITRLFGASSGYGKDGKCPLQARLIFRDSILSNGKELLDNGASSYTETKFENTINRVTAVANPRQIERAIRTSQFNFELIYDVVEETEVREDLETILDGFKLLELDYLGGNGSRGYGKIKFNNLSAETVFGDYDVTELNEKLGA

SEQ ID NO: 26 - protein, 299 aa, Streptococcus thermophilus
MTYKLYIMTFQNAHFGSGTLDSSKLTFSADRIFSALVLESLKMGKLDAFLAEANQDKFTLTDAFPFQFGPFLPKPIGYPKHDQIDQSVDVKEVRRQAKLSKKLQFLALENVDDYINGELFENEEHAVIDTVTKNQPHKDGNLYQVATTRFSNDTSLYVIANESDLLNELMSSLQYSGLGGKRSSGFGRFELDIQNIPLELSDRLTKNHSDKVMSLTTALPVDADLEEAMEDGHYLLTKSSGFAFSHATNENYRKQDLYKFASGSTFSKTFEGQIVDVRPLDFPHAVLNYAKPLFFKLEV

SEQ ID NO: 27 - protein, 299 aa, Streptococcus salivarius
MTYKMYIMTFQNAHFGSGTLDSSKLTFSADRIFSALVLEALKMGKLDAFLAEANQDDFTLTDAFPFQFGPFLPKPIGYPKHDQIDQSVDVKEVRRQAKLSKKLQFLALENVDDYLNGDLFENEEHAVIDTVTKNQPHKDGNLYQVATTRFSNDTSLYVIANESDLLNELMTSLQYSGLGGKRSSGYGRFELDIQNIPLELSDRLTKNHSDKVMSLTTALPVDADLEEAMEDGHYLLTKSSGFAFSHATNENYRKQDLYKFASGSTFSKTFEGQIVDVRPLDFPHAVLNYAKPLFFKLEV

SEQ ID NO: 28 - protein, 299 aa, Streptococcus equinus
MTYKLYIMTFKNAHFGSGTLDSSKLTFSADRIFSALVLEALKMGKLGAFLAEANQDDFTLTDAFPFQFGPFLPKPIGYPKHDQIDQSVDVKEVRRQAKLSKKLQFLALENIDDYLNGDLFENEEHAVIDTVTKNQPHKDGNLYQVATTRFSNDTSLYVIANESDLLNELMTSLQYSGLGGKRSSGYGRFELDIQNIPLELSDRLTKNHSDKVMTLTTAFPVDADLEEAMEHGHYLLTKSSGFAFSHATNENYRKQDLYKFASGSTFSKTFEGQIVDVRPLDFPHAVLNYAKPLFFKLEV

SEQ ID NO: 29 - protein, 280 aa, Limosilactobacillus reuteri
MTYKLYIMTFQNAHFGSGTLDSSKLTFSADRIFSALVLEALKMGKLDAFLAEANQDKFTLTDAFPFQFGPFLPKPIGYPKHDQIDQSVDVKEVRRQAKLSKKLQFLALENVDDYLNGELFENEEHAVIDTVTKNQPHKDDNLYQVATTRFSNDTSLYVIANESDLLNELMSSLQYSGLGGKRSSGFGRFELDIQNIPLELSDRLTKNHSDKVMSLTTALPVDADLEEAMEDGHYLLTKSSGFAFSHATNENYRKQDLYKFASGSTFSKTFEGQIVDVRPL

SEQ ID NO: 30 - protein, 301 aa, Streptococcus oralis
MTYKMYIMNFQSAHFGAGTLDSSKMTFAADRLFSALAIEAKKMEKMEEFVSLAGQDDFVLTDAFPYQSSPFLPKPIGFPKFDQQDLTTDVKEVRRQAKMAKKLQFIPLDKFDSYVKGTLFEDEEHAVTNIITKNQPHVDGNLFQVSTVRFRDDSSLYVIANESDLLNELMTSLQYTGIGGKRSSGYGQFDLTILDLPDSLKNRLTKTHQEPVMTLTTSLPVEKELEYAMETGSYLISKSSGFAFSTETNENYRKQDLYKFASGSTFSETFTGHIVDVRPLDFPHEVLNYAKPLFFKMEEER

SEQ ID NO: 31 - protein, 301 aa, Streptococcus mitis
MTYKMYIMNFQSAHFGAGTLDSSKMTFAADRLFSALAIEAKKMGKMEEFVSLAGQDEFVLTDAFPYKSGPFLPKPIGFPKFDQPDLTTDVKEVRRQAKMAKKLQFIPLDKFDSYVNGTLFEDAEHAVTNIITKNQPHVDGHLYQVSTVRFADQSALYVISTESNLLNELMTSLQYAGIGGKRSSGYGRFDLTITDIPDALKNRLTKVHQGTVMALTTSLPIEKELEHAMETGSYLLSKSSGFSFSTETKENYRKQDLYKFASGSTFCETFTGQIVDVRPLDFPHEVLNYAKPLFFKMEGER

SEQ ID NO: 32 - protein, 299 aa, Streptococcus sanguinis
MAYKMYLLSFQTAHFGTGKLDSTAFTFTADRLFSALFIEAIKMGKEEDFLSLVRQDDFVLSDAFPFVDMPFLPKPIGYPKMDKLPQMNDLVELRKQAKKTKKLSYIRLDDFEAFLNGKVLDNEEFGKLNIVTKNQPFMDGHLFQVRTITYQENVSLYLIAKQSQLFDELMTALQFSGLGGKRSSGYGRFHLEIKDVPEKLSKHLAFEAVSNVMTLTTALPNDDDLEEAICNGQYLLAKSSGFAFSNSYQSNFRKQDFYKFKAGSTFSKSFRGQIADIRPVNFSHPVWNFAKPLFYKLEV

SEQ ID NO: 33 - protein, 357 aa, Streptococcus thermophilus
MKNDYRTFKLSLLTLAPIHIGNGEKYTSREFIYENKKFYFPDMGKFYNKMVEKRLAEKFEAFLIQTRPNARNNRLISFLNDNRIAERSFGGYSISETGLESDKNPNSAGAINEVNKFIRDAFGNPYIPGSSLKGAIRTILMNTTPKWNNENAVNDFGRFPKENKNLIPWGPKKGKEYDDLFNAIRVSDSKPFDNKSLILVQKWDYSAKTNKAKPLPLYRESISPLTKIEFEITTTTDEAGRLIEELGKRAQAFYKDYKAFFLSEFPDDKIQANLQYPIYLGAGSGAWTKTLFKQADGILQRRYSRMKTKMVKKGVLKLTKAPLKTVKIPSGNHSLVKNHESFYEMGKANFMIKEIDK

SEQ ID NO: 34 - protein, 357 aa, Streptococcus salivarius
MKNDYRTFKLSLLTLAPVHIGNGEKYTSREFIYENKKFYFPDMGKFYNQMVEKRLAEKFEAFLIQTRPNARNNRLISFLNDNRIAERSFGGYSISETGLESDRNPNSAGAINEVNKFIRDAFGNPYIPGSSLKGAIRTILMNTTPKWNNENAVNDFGRFPKENKNLIPWGPKKGKEYDDLFNAIRVSDSKPFDNKSLILVQKWDYSAKTNKAKPLPLYRESISPLTKIEFEITTTTDEAGRLIEELGKRAQAFYKDYKAFFLSEFPDDKIQANLQYPIYLGAGSGAWTKTLFKQADGILQRRYSRMKTKMVKKGVLKLTKAPLKTVKIPSGNHSLIKNHESFYEMGKANFMIKEIDK

SEQ ID NO: 35 - protein, 357 aa, Streptococcus equinus
MNNDYRTFKLSLLTLAPVHIGNGEKYTSREFIYEKKKFYFPDMGKFYNKMVEKRLAEKFEAFLIQTRPNARNNRLISFLNDNRIAERSFGGYSISETGLESDRNPNSAGAINEVNKFIRDALGNPYIPGSSLKGAIRTILVNTTPKWNNENAVNDFGRFPKENKNLIPWGPKKGKEYDDLFNAIRVSDSKPFDNKSLILVQKWDYSAKTNKAKPLPLYRESISPLTKIEFEITTTTDEAGSLIEELGKKAQAFYKDYKAFFLSEFPDDKIQANLQYPIYLGAGSGAWTKTLFKQADGILQRRYSRMKTKMVKKGVLKLTKAPLKTVKIPSGNHSLIKNHESFYEMGKANFMIKEIDK

SEQ ID NO: 36 - protein, 359 aa, Streptococcus mitis
MKTEYRTFQFSLLAMAPIHIGSGEKYTSREFIYENRRYYFPDMGKFYNRMMENGLDRKFEQFLQETKPSARNNRLISFLNDNRISERSFGGYSISETGLETEKNNQNPRSGSINEVAKFMRDAFGNPYIPGSSLKGAIRTILMNTNPDWNNKNAVDFRGRSPKENKKMIPWGAKKGQEFNDLFHAIRVSDSESFNNEQIILVQKWDYSAKSLTAKPLPLYREAIAPLTKINFTITTTTKEAGILIEELGQRAQAFYKEYKNFFLSDFPENKIQPNLQYPIYLGAGSGAWTKTLFQQADGILQKRYSRMKTKMVGKGVLKLTKAPDKSIKTNKGERKLIINSDSFYEMGKANFMIREILQ

SEQ ID NO: 37 - protein, 359 aa, Streptococcus pseudopneumoniae
MKTEYRTFQFSLLAMAPIHIGSGEKYTSREFIYENRRYYFPDMGKFYNRMVEKGYDQKFERFLQETKPNARNNRLISFLDDNRISNRDFGGYRIVETGLEIEKNNRDSKLGTINEVAKFIRDPFGSPYIPGSSLKGSIRTILMNTNPDWNNKNAVDFRGRSPKENKKMVPWGAKKGQEFNDLFNAIRVSDSTPFNNEQIILVQKWDYSAKSLTAKPLPLYREAIAPLTKINFTITTTTKEAGILIEELGQRAQAFYKEYKEFFLSDFPENKIQPNLQYPIYLGAGSGAWTKTLFQQADGILQKRYSRMKTKMVGKGVLKLTKAPDKSIKTNKGERKLIINSDSFYEMGKANFMIREILQ

SEQ ID NO: 38 - protein, 354 aa, Fusobacterium periodonticum
MKTEYRTFQFSLLAMAPIHIGNGEKYTSREFIYENGYFYFPDMGKFYNRMVEKGYDQKFERFLQETKPSARNNRLISFLEDNRISDRNFGGYRIIETKLETNNNYLRGGALNQVSKFIRDPFGNPYIPGSSLKGAIRTILMNTNPDWNNKNVLQGKKENKSLIPWGAKKGQNYDDLFNAIRVSDSKPFSNASLILVQKWDHKVKPPIPKPLPLYREAIAPSTKISFTITTTTKEAGVLIEELGKRAQAFYKEYKNFFLSDFPENKIQPNLQYPIYLGAGSGAWTKTIFKQANGILQERYQNSKTTRMVEKGVLKLTKAPMERVKTTQGPRKLIMNDESFYEMGKANFMIKEITK

SEQ ID NO: 39 - protein, 364 aa, Streptococcus oralis
MKTEYRTFQFTLLAMAPIHTGSGDKYTSREFIYEDGYFYFPDMGKFYNRMVEKGYDQKFERFLQERKASASNNRLISFLEDNRISDRDFGGYRIKETGFETEKNNIDSKLGTINEVSKFMRDSYGNPYIPGSSLKGAIRTILMNTNPDWNNENVVKDKKENKSLIPWGAKKGQNYDDLFNTIRVSDSKPFRNDSLILVQKWDHKATTPLVKPLPLYREALTPGKIINFKITTTTKEAGELIEKLGEKAFEFYNDYKIFFLKDFPENKIQPNLQYPIYLGAGSGAWTKTIFKQAKDILQERYENSRTTRMVEKGVLKLTKAPMKSVKTTQATRKLIMNNESFYEMGKANFMIKEITNDNFNFSSR

SEQ ID NO: 40 - protein, 534 aa, Brettanomyces bruxellensis
MGLSEFEKRRQQNIQRNQELFKKLNLDTLSSDFHRSIGETKNGVEKPKKRKTRKSTRKKKELRPEHPLRRSARLAGIKAESTDDNKLQEQLEKQRKEKEEEERLKSIRLSGDLMLTDVLNSKGKAGEDAKETLDRLSRLGKSFSMGDFFEAVKSRKQGSKQVKQLREEFDSMKLYERFHPNDIKLTSQRMTYVMFHPTVDRKIVMGGDTNGLMGIWSLEDDTEEDGPAITQFKFHGKNIPKLIVRPEMPGEVVSCSYDGSIRVLDIEKKISQSVVEFDDQWGDASGISDTNFIDKNVCYFTTLGGEFAQFDLREKSKIDRNALNVLRLHDKKIGGFAVNPSFNQQIATASLDRTMRIWDLRTVKESQWSEFENAKSPHCMGAYHSRLSVSIADWNLSNDVVCNSYDNTIRIFQLGNQITDDPGYIFKPKLEDSPEGLESIPINLKPTNTLKHNCQTGRWVSILKARWQQNSRDGVEKFVIGNMNRFFDVFDRDGTQLAHLGDDYMTSVPAVASFHRTENWIVGGNASGKVFLFS

SEQ ID NO: 41 - protein, 984 aa, Colletotrichum lagenaria
MVFCAYCGKSFTRKEHLERHIPSHTNVKPHRCSACQLSFARRDLLQRHHSTYHEARDPMEPLPGGVPTVAGRTPIACQNCANAKTGCDKRVPCSRCAEKNLPCAARFARRSSKAAVRAAQASAAFQQQMMPTVPQPPQPAQPVAFMDLDPTLPKPDDPSKMSTGSPIATMDPRMVENTMRKSSPPHSHRSPDSFPSPHSRVDGLDEFMQLNNDFITPDTNYQDMLVWPEYPLDLDIYSNQLPMTRPDIPMPSFTELSDMSSTSASEPMTASSSRGSIHTRSTSIMSTAEFDTTMKPAESSVGIVSDSMIPEFEVVIAAEDSWPLARCNPPIYSGTCPRTAIVHLECLERKSKEDGTWSSLEKYLEKVDWDSTDASVVPLTSRTRDRMLAITQSFLQKALEIHRSGLNNGFSKTGYATPSEFNFIVLPPPKILEYFLRSYVRSLQYYYPLIVAGCVDPNEMLLNNQASTLLVLLMIAQGASNVPMAEARYLAAGLTETCRISLFDIIEKDVELSADPIALRCALLFTLLGAWSGDKWLMDISMGQRGMYLSMLRHAGMLEPQPSMIPTLNSTTSTELQWRAWLHREMQNRLVYNWVMCDQELSLFHDTPPQLAITDLQCPLPSPEVLWLASNSEQWFANVQNIYGCTANVNPQLLSSPSLTPSLCELFQDFIHDNLTRRQGSLSPQQLRLLLHPLQALLCHLRQMFSCFSDVLSTRRATSRTVTKASTMLRLEEVQALLQKWYELTMSSSKNDPDCGTTRCNLVLYHLISLNAVTNFPEIERLARRDGFDGSFWELSLRHKRCVYNREEAIFHCGQVLRLLRSMPADRRPCWWPAGVYRATLILWADSVSRLDPNFHKAQAQQQSSSPASQHQDHRPEELSGAGASNAATSGQSFGSGNIVAIDQVTPEDPAVISYLWSGGDGTPVLTRRDGTTLGLDKPSEVIGYGIMSIEEGVTCRVGDGIKRKLITLNSNWNVDGLGATTTA

SEQ ID NO: 42 - protein, 512 aa, Kluyveromyces lactis
MGELTEFQKKRLENIKRNNDLLKKLNLNNVSSQIKREAGVEDEHLNRKRKKKASTAKKAVKKEPKPAAIPTRRSRRLRGENVDGNGIPNVNDNQLLKMGQSDSTPELEALDELKNTALSGDVKLSDLIKSENEEELLDKFKSFANKNFSSGDFFKELQQQQVPTPEIKQLQEDFDLKLYDIFQPNEIKLTAERISATFFHPSVDKKLVICGDTAGNVGLWNVRENQPEDELEEPDITKVKLFTKNVGRIDTYATDSSKLLAASYDGCLRSINLQDMNSEEILVLKNEYDDPLGISDFQFNYNDPNVLFMTTLSGEFTTFDVRTKPTEINLKRLSDKKIGSFSINPKRPYEIATGSLDRTLKLWDTRKIVNKPEWSQYEDFASHEIVATYDSRLSVSAVSYSPMDETLVCNGYDDTIRLFDVSGTLPEELQPKLTLKHNCQTGRWTSILKARFKLNMDVFAIANMKRAIDIYTSSGVQLAHLPTATVPAVISWHPTQNWIVGGNSSGKAFLFT

SEQ ID NO: 43 - protein, 522 aa, Saccharomyces cerevisiae
MPELTEFQKKRLENIKRNNDLLKKLHLSGVASQIKHEAGVLEKSRAPAKKKQKTTNTRATKSASPTLPTRRSRRLRGESADDVKGIPNVNDNQLLKMGSPDGQDKNFIDAIKEKPVIGDVKLSDLIKDEEESALLEKFKRFNNGNFSSGDFFEEIKKRQGDVTGIDEFDLDLYDVFQPNEIKITYERISATYFHPAMEKKLIIAGDTSGTVGFWNVRDEPLADSEEDRMEEPDITRVKLFTKNVGRIDCFPADTSKILLTSYDGSIRSVHLNNLQSEEVLTLKNEYDDSLGISDCQFSYENPNVLFLTTLGGEFTTFDTRVKKSEYNLRRLADKKIGSMAINPMRPYEIATGSLDRTLKIWDTRNLVEKPEWSQYEDYPSHEVVSTYDSRLSVSAVSYSPTDGTLVCNGYDDTIRLFDVKSRDHLSAKLEPKLTIQHNCQTGRWTSILKARFKPNKNVFAIANMKRAIDIYNSEGQQLAHLPTATVPAVISWHPLRNWIAGGNSSGKIFLFTDDSGTIKQEE

SEQ ID NO: 44 - protein, 523 aa, Saccharomyces paradoxus
MPELTEFQKKRLENIKRNNDLLKKLHLSGVASQIKHEAGVSEKSRAPAKKKQKTTNSRATKSASPTLPTRRSRRLRGESADDTKGIPNLNDNQLLKMGSSDGQDKNFIDTIKEKPVIGDVKLSDLIKDEDEDALLEKFKRFNNGNFSSGDFFEEIRKRQGDVVGMDEFDLDLYDVFQPNEIKLTYERISATYFHPAVEKKLIIAGDTSGTVGFWNVRDEPLGDSEEDRMVEPDITRVKLFTKNVGRIDCFPVNTSKILLTSYDGSIRSVHLNNLQSEEVLTLKNEYDDSLGISDCQFSYENPHVLFLTTLGGEFTTFDTRVKKSEYKLRRLADKKIGSMAINPLRPYEIATGSLDRTLKIWDTRNLLEKPEWSQYEDYPSHEIVSTYDSRLSVSAVSYSPTDGTLVCNGYDDTIRLFDVKSRNHLSAELEPKLTIQHNCQTGRWTSILKARFKPNKNVFAIANMKRAIDIYNSEGQQLAHLPTATVPAVISWHPLQNWIAGGNSSGKIFLFTDDSRAIKQENE

SEQ ID NO: 45 - protein, 254 aa, Verticillium alfalfae VaMs.102
MQRLEEVQALLQRWYEISMVTYKANPSCETTRCNLVLFHVISLNGVSSFPEIERLARREGLNGTLWELSMRHKLCIFSLEEAVFHCGQIMRFIRAMPSDRRPCWWSAAVYRATLILWASTVTTPNPTLQTALQVRNSSSSPVSSDVDEERRQESTASKDHSLVDASVVIDQVTPDDPCLIRYLWSGGKGIIMVTRRDGRTVGLESPTAILNYGIGVIDEGFTSRIGDGIKRKLASLNTHWRVESPDVPVAPGMP

SEQ ID NO: 46 - protein, 345 aa, uncultured Phormidium sp.
MRKLLGDKINLQITGPFLCRDETLYFSRPIGFVGSTPLVPLDWDENHHLKKQVLWDKQQPCPLVKPSDRNKEDERKDDEIGNYKFRKYLPFTTVQNYLESGKINRGDWLTEDKTKTEIKPWLVETRSHNAIQEGTRQVKDADGYFVENAIRLKEGWSLAIALNQAIETPAIIQLGGEGHRAILQRVEALDNQWQKLQDLSMKNFTKGQESFKEGRKEDGKSIAYLVTPGVFERRDSKDKRLSLCSAWPSEWKLAHAVNSNQKIGPLVSVSTASALPISCRIREKDAPEKSIPAPQVFAARPGSLYYLNQPQELFQEVTETKVNVWRQLGYSELFWINYKGDKNHD

SEQ ID NO: 47 - protein, 998 aa, Sulfurisphaera tokodaii str. 7
MSKMSLKPMSEIIKEKFAALLHDPPNKPFIFSLNAFNEEKRISHVKVAKTLISHLDFLGKNELDEISSKIYSKKEKGCNSKVSDADSLASKFDRYLTTLIYQERGRKQQAMFANFKEIVMKNFLAPELEVNLSAIPSDAYHDPSGKDIKEFFNYLSEVFKKLGLTGVSIETYNVFYFFYEFLWVAKGYTVGPADTRVPTHSIFDHLYATASIINWFLGSTDLLLGLDIVGVADYINKSRKLRDLWASSYLVSALIWYVLISFVEKFGADSVLFPSLRFNPFFATYIYHKYLKNKEKDELIKEIVNYITTYIFNGDETYKKLGIPPYPIIPARATLILLGVKYVSRELGLKKSDISCIEKYVITRFNEGWGRLIDHLITYALSHTDNPFWALFNKVIEKESVKNAVKIPPVQLRVITLKKENEVSNYEEFDNRYRELVSNFKRKKLSKVSPHTQLSNYNYYIDSIGETKRGFEYCSICGVLPAMLILPKDENEYKKFVEEHTGKQFNDDQIEALKAILSPGEKLCPWCLIKRAIGVRPEFLRVLITSEDLRSFEEKDVFIPSVSHVAFYKKFEKLKEDNIINPDKENTISLWKYYETYPIEKRGLLRENPEEKGWKDVSPYYALVRADTDYLGDLLEGKVTPYLAGIIDSSFYGGERENESKVKNAITRYLRNAAKGLYQEVINDVLKEDINQAKELIKNAAVTAEIEINDNDIERIISDLKDFLNKNVDRDRLLLFPSWHVSISSMLNRILLKEIQLVNALGGFVVYAGGDDLLAILPVENALQFVENSRKIVAGIEDASSYKGFIKINNSYFSQLPLVGRSYILYFSHVKYPLQLALEESYNLLEEGKERVKYDKYKKDIVIFKYRNSVSFIPLSLIRPYEENNDNSIKRYLDNLGKSLELLRILYDAIRQKKLSKSLIYDALADTILKSRELESEILVKYLDYLVDKNKIDKSFSLSFVNYQDILKISIINGETSEPLTYNLFKALSIILGAEKIE

SEQ ID NO: 48 - protein, 1012 aa, Phormidesmis priestleyi Ana
MSQFLASKVFWQSKIWGLLHNPALQVLYDAAEPSRQLDWQTLPVMEDWQTKGWDPATSTQPTLKRIGLARAIAAASDRAALSNLSTGVNYASSDSGETSPQDNSGLEISHLLSGEKQPFQIEQHQQLLAQSSRADYLDGIGSHLLDPVRKMTCPQQVFWWLWRCLPEATCQAMGNDPMLLLTPAEPRLPDSSNWGNASVTAAMAGALAGYNLSLEDLSTGTDTEDSTAYLATFSFTPIQELIKASRKMRDFWAGSWILHYLSAKVSWALAWKYGPDCLIYPSLFQQPLIDQWLVQGIGNQQGDFKGWPEWQQWIQPPTEGQLLTAGFPNVLVVLLPKDRVQAAMQMAEQTLRQEWKKLSKLVFHELKARHWMKDLSFDSATWHGWLDSQWQTYWSALPVGAEGESLQSNASTGQADALQTWIQAQNKMYQVSDRAASKSRLFQDAELTFLRQVCDLGKQGLDQDATVNVGSWWAPIFDQTRWSLTAVKSFRNWEIPTVFSVRSTVSGVGAAVHSEQPWVPEARVKAQWQRQAGLFDGNEQLNATETLKRGLHLILPKLLQLEDSKLNAAYPDLTAGVAGYLKVKGDEHLEYFNQVCHDLSDKVSEKGNGIDRQAWGIPWIDDFADADYRRYHPRCLNAGWLVEELDISQEQATTYRQALQSTIDQHYPGRSPADWYVLAAGDGDGMNEWLKGQKLSTYRDYVLESRSVPASVNTAFEDFLDVKKRMGPSTHNALSRALLDFSNQLVPYLTEQRYAGRLIYGGGDDVLAYSNLWEWDSWLWDIRQCFRGEKDEAQAFENDGDYWRWKDPQTLPQGLSSRPLFTMGKKASISFGIVIAHHSVPLAIALEQLWAAESDGAKKHLSPTGEAKDAVQVRVLYGNGNQLQATGKFGVKGECRTKGKLGMFAQWRSLLQSYGSTPLEPALFEQAAQMWSQHPVPSIDAISPWTQAFCSRREALQEDSLKQAFQANLAALLTSLWETTMPAQRDLEVQNWLKLAAFMLRHREITLQGRNR

SEQ ID NO: 49 - protein, 560 aa, uncultured archaeon
MSNIILAEIPEEIDPASIDVNAQKAAKKEWKRSAGKAKALARQAICHDVWIDQLDDVIEFNSAWVFPKDEENYSNTRKRLMRLLAGRKSIRDFKPAKGRLGIPKSSLDGARESVLNKNEDLPKWLALRMRLSNGEQLCAVGLTKRLGGGRVPFPSVIRVAADPWIRGIRSGRRKDANQLLDEIRDLCRVSCKLQGRSECDYQPLDQNCRLCKGENSFSSGTGKRLYQDFPFDGQIFYPSRLYRVKKDLKDISMDTSEDNPYKDDMKKLIQIEEKVRRLQKKDNGGLGLGEPNPYLAILVADGDHMGKALSTIKSAEEHRKFSSNLSTFASEARTIVEGNHGCMVFSGGDDVLAFLPVDTCLKAARDLHDKFGELQFSDKNKNRTRLTLSVGIAIVHSMEPLEDLLVFGRAAEQAAKNPDRNGLAVHLRTRSGGEPILIREQWKESSENGLDQRLKSWAEMHLKDELPDKAAYDLRQLAEEYRGWPEISHDLLEKDMRRLLKRKRAGQGTKDISDVDLTELMKDVKSHDDLIRRAKELVLARSIAEAKKIVQASISKEASA

SEQ ID NO: 50 - protein, 512 aa, Porphyromonas gingivalis
MRELLERLPKEERPEGSFEVLSPHRDGKDVPEETSKKVGLFPDRLFAKGELEEEKLQEIKGKIFIELAEKFKNAFQDEKDGITRKIDTESDPKRKRELEEIQSRYSNAALKGEDIRKYLENYLSISCITVELSSDCNILEQLNSYLDTQELFNKASIQTDEDYMELFIEAPNNTFLKAYSQDRAFPSTSEIAVSGWEQDPPKDENGEVEYTQLTSKPGFRNCYKYLAVIKADGDGFGTYIKELKVDEEKEIKENGEKENELSRFTKSFFEFSVEVANELIQKTKAIPVYIGGDDLFLFAPVLEGNVEKDVFNLIKEIDDLFIQKEIGEGLSMSYGVSIFYYKSPMSEAIDIADSMLRKAKEEKEEKEKKKDAVAISIQKHSGQKIEFLLPCKHSSADETDRKESLYGKTVELVRAFKGDGNMLNSLIYWIEEMYEILFIEEVTQSQERINAVFDNFFDEEIHKENEVFFDLLKDFIHSMHRSEDAPRKLSDKKELLHGILRYCQFVTAKKEK

SEQ ID NO: 51 - protein, 276 aa, uncultured Phormidium sp.
MINYVYLYLLSPLHTGGTTQEGNLLGIARESHTNLPYIPSSSIRGKVRSSVGNRVTRVQLFGPELKDTQDADFVFEYEYETDKKLNQLEQGSIWIGDASILWLPVPSLSHGVVWISCPFLLQRWARFNSGDVHIEEYSSNLPKKSSVYLKDAIIKGDRLKPFANWQDFIPQKDSSIKRVLVLPDQHCATLIQMSLWRQVKVKLDEHKVVDGGFRYEEAIPPDTLMYFPWGITSQANGTAKQSRDEFEKILAAQPVLQIGGQESLGRGFVQQWTITK

SEQ ID NO: 52 - protein, 317 aa, Saccharomyces cerevisiae
MNFSLSKQSSEKQSSYTDKSRSPNIGMCTVNYKSNLPLQVSAVDQLSTGKGTQFYGHNSYKNERECYNSTKINLPPISSLLPNFENNTPPNVDSRVQFPPQQVYQSMNVVPIVNEIYTPISMNATSDQYPIYYTESQQPIPHSQSPHLTSSAPLMMPVMVPTVYKPLTPYDKEPITIASEPNFTAISMASHPNAALELCHDRPKSVPPGYGVLPTMQEASNGRTKSEPGAVLNGSATFSDWKTDTRISSTKLRKQCPVCGKICSRPSTLKTHYLIHTGDTPFKCTWEGCTKSFNVKSNMLRHLKSHERKRNKVLNTT

SEQ ID NO: 53 - protein, 317 aa, Saccharomyces paradoxus
MNFSLSRQSSEKQVSYTDKSKSPNIGMCTINYKSSLPLRVPAVDQLSPGRGIQFYGHNSHKIEQECYDSSKINLPPISSLLPNFENSTPAKVDSRVQFPPQQVYQSMNVVPIVNEVYTPISMHTTSDQYPIYYTESQQQIPHSQPAHLTPSAPMMMPVMVPTVYKPLAPYDKESITIGSEPNFAAISMASHPNTAVELCHDRPKSVPPRYGVIPTIQEGNNGRTKSEPGATLNTSAAFSDWKNDSRISSTKLRKQCPVCGKICSRPSTLKTHYLIHTGDTPFKCTWEGCSKSFNVKSNMLRHLKSHERKRNKVLNTT

SEQ ID NO: 54 - protein, 410 aa, Bacillus safensis
MPEKWFHIQPLDPLLIRDARPFQSVPGIRARSLNDVPPSVVAGTLRTALAKKRQSIKHTTPLSYYSKSLVRGPLYRWHDHLYVPIPNDLELFDDEYGMNQVRPKPMMSEGQGFFGTGKTGRFSEALWPPIGAGARKKMKSPPAFVSLKWMVNWLTDAVDESQVKEALTEYQHMQKENHAFKKETPPAFIPELQRQERTQIEVETGSKKAKDQHLFSTESLVFPDGFKLEAGIELSTEESEALKPFSSLHPIGGERRLAHIEDRPHVYQQDWTCPDMIKESLKNKPYVRMILATPAYFSKGWQPGWLDEQLMSKPLFDTNVRLKLVWASVPHWQPISGWSLANKDKGREKAIRRLVPAGSVYFFTVEEGDPAELAEKMWLQSVSDRNRRKEAFDREDGCGLALWGTWKPNE

SEQ ID NO: 55 - protein, 405 aa, Porphyromonas gingivalis
MSTYLIKLTPLDKFFFGQKKTFGDDNANYFVYSSHFPQQTTLLGLLRYQLLQIAGEEVFKDNKIQDEHKAGELIGKQSFSPFVKDKLQFGIIQSLSPVFIIDKKKDEGKEEYFLPVGKRFQKKKEKAPYSLLHLSCEDECPPIFESRESQKEKEEAYDPKKGLASCWLSSKSHTLLNEEDFFTKDERIGIRKDYEGATNDDAFFCQRYYRFKNFKKVVEDEEAKVCKQPPVRVHDFCFATVLETEKAIEHEKLNGRIVYLGGERQPFLMEVSKEEKASDLTIGSSTLTGDENHYTVVLLSDAMIEPDHLRKVKFASTEVKDFACLLTHVGTQKFYNKENKRQEQYLKKSATALSLQKELYARGSVFYFDTKEQANQFCEDLEKVPNFHTIGYNHAIIIEPTKTKE

SEQ ID NO: 56 - protein, 148 aa, uncultured Phormidium sp.
MDLYSSGQLLNNFKGVKMMVFDPRTIATPVYEALAELRQQHNGNDSRLKEQKNQAVELYTYLSTWGMMRLKAEEQALTQDGKKDVVKKYFDCLQRLSGMQNLSNNQGLKTLIAMDIDEYLGITGLGLAIAQEFSFWATAVYHDIKGEN

SEQ ID NO: 57 - protein, 297 aa, Caldibacillus
MYSKVKPFLLHAITSVHPGSGSEVGLVDLPIQREQHTGFPKIESSSLKGAIRYTVDSILKENEDRHKLELVFGSDLSRGNENGTIASAVAFSDARILLFPVKSMRGIFAWITCPEVLYRFNNDMLLNQTGVEPLPVPEPNTVSSEKVLVSPGKMILEEYTFKVHKEEETEKLAKRFSSFFEGPVTIDIANRLVVLEDDEFKDFVRLSTEVNARIKINTETGTVDKENGALWYEENIPPETIFYSLLYIGKVRSRDNKGFETIEDVEQFMHNSQYFPDVFQLGGNSTIGHGMIRKIWL

SEQ ID NO: 58 - protein, 295 aa, Anoxybacillus tengchongensis
MYSIVQPFLLHAVTSVHAGSGSEIGLVDLPIQREKHTGFPKIESSSLKGALRYHMKLSANGNKDEEKKLEIIFGGEKSDENNTVASATALSDARILLFPVKSMRGVFAWVTCPQVIQRWNYEMSMHKKDALALPVPKENTCSTTKLLVGKSELVLEEYTFSVQVDENTKAIAQRIEQWLGAHVCIPVVDRLVVLSDDDFADFVQLSTEVNARIKINDETGTVDEGALWYEENVPPETIFYSFLYVGNPRKKDERVKDAEQIHTYLVAERKFPEVFQLGGNSTLGRGMMRTIWLGV

SEQ ID NO: 59 - protein, 241 aa, Porphyromonas gingivalis
MKTAAYMIHCLTNMHVGKGDATYEVVDKCVQRDVTTGMPCIYSSSLKGALRQFFSKQGFADIKYVFGSDVSRDVKDESKGNESDNSNEENPKEHSVGNFTFFQANLLAYPVQTTQEGATYILKTNKALQESIKEFAKLLSAPSNIEDLLPEGIEKDLSNEALEQLPVIARNQLDNGESKNLWYEEVVPAESRFVFFVSYDDEEIFVEFDKIIQEGVIQIGANGSIGYGFCKIANVTNPFKK

SEQ ID NO: 60 - protein, 289 aa, Bacillus altitudinis
MEANRWFWIHCLSPLHIGTGEGVGVIDMPIQREKVTEWPIIPGSSIKGVQREHYRAKGQDTFVQQVFGTQGDQDGSAGAVVMTDGRLFSFPVASRYGTFGYVASPLILKRLARDAKALGVSLDIPDLIEESSTLVTLNSTLVENDELYLGEFQGTAKKSQQLTDFAKKMAKLLFDDKQSQTLWIERFVIVSDDVFQYIVTMNSEVTPRIRIDHETQTVSKGALWYEEYVPAEAIFYGLMWCDRVDGSNDLSVRHQLLNQLEKQMHLQLGGNTNVGKGRVSWRMTGGERS

SEQ ID NO: 61 - protein, 352 aa, Archaeoglobus fulgidus
MFEKAVVFGLYSITPVHAGSGAELSVIDLPIQRERHTGFPVIWGQSLKGVLRSRFRQLELDEKIEVESQKWKWKEKTKEVLKEKADEFIKKVEERKRDPLLTEIVFGPATDGASEHAGAVSVGDAKILLFPVRSAKGVFAFVTSPIVIQRLKEDLELVSEIENDIELKQILSRFKVELSNNETIAGNALILNGENKVILEDIVLKVKSDSNVIENLVEVLKTLFGDNFFGKPIESIKERIAIVSDDVFKSFTRFSTEIVARVRIDAEKGTVARGGLWYEEFLPSDTLMYSLIAVGSPKKENLPKEVDNTQKIVNVLKVTFNNAFLQIGGDETVGKGFVKVRAGVLTGGHKGN

SEQ ID NO: 62 - protein, 149 aa, Clostridium botulinum
MYQTINIKDLKRKKASFAFNAIEYIQSSGDKKFKGYIRNIPMYIYTNGLIATIAFILKKMGTEYEREKGTDKQSYYEIGEILRDYFNKDFIEYDDKFKEKSLEDIMKELIKCDSKSYRRITLDILSILEWLVRFSDAMLEGEGESSEPI

SEQ ID NO: 63 - protein, 118 aa, Arcobacter ellisii
MSKKRIESYIPKAIELLTKEFPEGKIASSYNGYISSFGASIIQSGLKPTLAVFENTNASTKEDKSYLTKLILKLLPNTQGNSLLKFVLANKQKEESLKEEILDIAVALKLSIRTFKLV

SEQ ID NO: 64 - protein, 137 aa, Candidatus Frankia californiensis
MSRRDQGLARDTWLLVEQVCAEADAPERREFRTRAMRLGPMLRTSGLAATAAFLAAKASGTGSLPAAYRRMAEALARYVLASVGQQPMDRNGFVLWLGRADPATYRQAGAAAREFGLWVRRAAEALIPDAAPARGDQ

SEQ ID NO: 65 - protein, 124 aa, Neisseria elongata
MPHIRSQKYAQRALGLIQKVKEKNEKVKEYRTLALNFPTMILQSGLAQAIGFLQAKGKEEHLLLLAHIAELLGENKDGLHKKILEADLSHYQLLTRQAIEAASGLKRYTQALLPKPKDEQGGQS

SEQ ID NO: 66 - protein, 135 aa, Vandammella animalimorsus
MNPSNPSTALRTMEQERAQFALARIQAIAQESESIQAEMRRYLNGLPALIHMNGLGQALAFYRSQGNGSTHDRIYRILGDWLCGADSKGRIFDEADDLLTAITRSDMPHYMAAQNEAQALLEWLKKFALALLKKA

SEQ ID NO: 67 - protein, 526 aa, uncultured Phormidium sp.
MTNTQTSSNSVNTLIITVGTRQIGWNCKDGVIRSFGADGNIGYPRHVDELYQEIEIPRGQYQEKDKTHPYSAKDLGERYYDRCIYEFSYDFSRVELLLDYQIIEAGVKQGLKHIILWGTDQPDNVSWFYRRLDTIWLAKLMEAKIKSIWPNLKVDVHTPKIEANDNSAIREELEQLILREALDFFSPTGIDEQFVLWIQNKGCTPAIASGVEICAAALVRQCQVFNATPDDPESFFAPHPSGAQFACHSPGYKLIPMGEYFWPLERVRVISAWKRGDFSEAQIWLKLHQNRYSLIYKLAGILALSANWENDKFFTKVGEWVRCRDVLKSVGESQVKAWEEQLDRLKNNQPTQAWESSFLIELPLYRQNYTAAFIQFVQTLERLLYLHSKSGNWLGKRYITIPAHLTHLGDGYQPGLSGLIDGWCQSKKFNKNHKWYKLLHRLRDKRNQVIHSGESVTLANIRSLWSDEGLFPVKHSEDPQVIRDLMVEVLKETCDRTWEIPQKTFLRSLYDWGLKTLNDESASAKN

SEQ ID NO: 68 - protein, 244 aa, Thermotoga maritima
MVKRMKLPESKNLSLYWEKYSFLLLEGSNSKDKEIVSKIFQTVEKLQSDFGKISSILKERREQLIKKRKPFINLKMRVKGKLLLGAGNPSSIEVGITLSRNYGLPIIPGTAVKGAFASFLFEFERDKYESLAHIFGDTEREGDLIFLDAIPVSDLKFSLDIVNPHFQPYYMKEKLPPNDWYDPVPIKYLVVSSGVFWFTVLESRSGVIGNSEKELIKQRFVEMLKAYGLGAKTSYGYGRFEEPS

SEQ ID NO: 69 - protein, 304 aa, Aquificae bacterium
MDRRESVDENLALLLGKRIFDYLKDDYQRSGKISSSMLKNLWNNKLKSYSYPKEISEVIKSHVDSIAKSSSCHVVFDKTLQTASPLVCGLGATHIFETSITLHHIWGVPYIPGSSLKGVCRQVVFWKLVEVGKLSKDNLKDSQEKFYGDLNTDDKEILKYQLLFGTQGFKGLLLFLDAYPEIPEGSMGKLFRLDIMNPHYSEYYGEKRDIPGDWENPVPVFFLTVKEGVSFRFVVLFDECRWEVIKKKGIPDKEGKRRQLADEEIKGVEEYMESEKFYEDIIGQALEFYGVGSKTRLGYGLFER

SEQ ID NO: 70 - protein, 246 aa, Porphyromonas gingivalis
MNFGHWYYREYFDTINLDSKGIVTNFSTFNKGKNDKLIKGATLPPYDKENDIEDVTSFELKTCYPGLLCGVGYHHEINNPKNEPKEDDAPEVYNLGMYFDYTSGLPVIPGSSIKGMLRSAIEEWDFLADYKPNNGVTREEIIEKVFVGKEYSIYDRDIFLDAIPIRVDNTLFGEDYITHHPNPLQNPNPVRFLRVEPGVTYQFRFILKDHGEKLTVDFKTKLFKAIICTFGLGAKTNVGYGQFVEP

SEQ ID NO: 71 - DNA, 31 bp, synthetic construct
  source 1..31: mol_type = other DNA
gttgtatcgggaggcagtaagaatcatcttt

SEQ ID NO: 72 - DNA, 28 bp, synthetic construct
  source 1..28: mol_type = other DNA
actagcattaattgacagctgacccagg

SEQ ID NO: 73 - DNA, 27 bp, synthetic construct
  source 1..27: mol_type = other DNA
gcttaggaggaggaagttctccaatgt

SEQ ID NO: 74 - DNA, 31 bp, synthetic construct
  source 1..31: mol_type = other DNA
tacatcaggccttcatcctgaggattttatc

SEQ ID NO: 75 - DNA, 35 bp, synthetic construct
  source 1..35: mol_type = other DNA
gtcaagaaagatcttaagactggtcattcaaaggg

SEQ ID NO: 76 - DNA, 31 bp, synthetic construct
  source 1..31: mol_type = other DNA
gataaaccagtcaaatctaaactggggagca

SEQ ID NO: 77 - DNA, 27 bp, synthetic construct
  source 1..27: mol_type = other DNA
ttaagcacctccgagaacttctcatgc

SEQ ID NO: 78 - DNA, 35 bp, synthetic construct
  source 1..35: mol_type = other DNA
ggaactcattaatatacttaatgggtctggagacg

SEQ ID NO: 79 - DNA, 26 bp, synthetic construct
  source 1..26: mol_type = other DNA
ggaagaaaagtcagaagaccaggacc

SEQ ID NO: 80 - DNA, 33 bp, synthetic construct
  source 1..33: mol_type = other DNA
atattgatgccattagaagggtctacaccagat

SEQ ID NO: 81 - DNA, 40 bp, synthetic construct
  source 1..40: mol_type = other DNA
atggacaatatgtctattacgaatacaccaacaagtaatg

SEQ ID NO: 82 - DNA, 26 bp, synthetic construct
  source 1..26: mol_type = other DNA
ccagaacatcatccctgcctctactg

SEQ ID NO: 83 - DNA, 28 bp, synthetic construct
  source 1..28: mol_type = other DNA
gaaaagcacacagcaaagacaaagaggc

SEQ ID NO: 84 - DNA, 27 bp, synthetic construct
  source 1..27: mol_type = other DNA
gctaccttcatcaccaaattgcactcg

SEQ ID NO: 85 - DNA, 26 bp, synthetic construct
  source 1..26: mol_type = other DNA
ctccatctgcaagctccatctacaag

SEQ ID NO: 86 - DNA, 35 bp, synthetic construct
  source 1..35: mol_type = other DNA
acaattaggtgggcttagatttctactgactacta

SEQ ID NO: 87 - DNA, 32 bp, synthetic construct
  source 1..32: mol_type = other DNA
cttagaattaggaagtttgcagtcacaccatc

SEQ ID NO: 88 - DNA, 28 bp, synthetic construct
  source 1..28: mol_type = other DNA
gatacaaggctccatttcatcagttgcg

SEQ ID NO: 89 - DNA, 36 bp, synthetic construct
  source 1..36: mol_type = other DNA
ttgaaactctcttcaagtctaaggactatgagttca

SEQ ID NO: 90 - DNA, 29 bp, synthetic construct
  source 1..29: mol_type = other DNA
cttcaactggtatctatgaggccctagag

SEQ ID NO: 91 - DNA, 33 bp, synthetic construct
  source 1..33: mol_type = other DNA
ttgatcaaatacacatcatacttcccagcagag

SEQ ID NO: 92 - DNA, 31 bp, synthetic construct
  source 1..31: mol_type = other DNA
ctttgcaaaataatggcaaagccatttccag

SEQ ID NO: 93 - DNA, 26 bp, synthetic construct
  source 1..26: mol_type = other DNA
ctgaagcctcccatccaatgttctct

SEQ ID NO: 94 - DNA, 28 bp, synthetic construct
  source 1..28: mol_type = other DNA
ggaaatgagcttgacaaagtggtcgttg

SEQ ID NO: 95 - DNA, 30 bp, synthetic construct
  source 1..30: mol_type = other DNA
aatggaatgaatgggctataaatagccgcc

SEQ ID NO: 96 - DNA, 25 bp, synthetic construct
  source 1..25: mol_type = other DNA
cttgtcccatctcacagaaggcgag

SEQ ID NO: 97 - DNA, 32 bp, synthetic construct
  source 1..32: mol_type = other DNA
gccacttgaacactgcgacagaactggatccg

SEQ ID NO: 98 - DNA, 32 bp, synthetic construct
  source 1..32: mol_type = other DNA
agcttccttcaccaaatcgcactggctcctgg

SEQ ID NO: 99 - DNA, 32 bp, synthetic construct
  source 1..32: mol_type = other DNA
ccggatgcatctgctgtggactttttaagatt

SEQ ID NO: 100 - DNA, 32 bp, synthetic construct
  source 1..32: mol_type = other DNA
ggttaggatttttctcattctgaatagaatca

SEQ ID NO: 101 - DNA, 32 bp, synthetic construct
  source 1..32: mol_type = other DNA
cttgtgtttcatattccgtaaaacgaacaaag

SEQ ID NO: 102 - DNA, 32 bp, synthetic construct
  source 1..32: mol_type = other DNA
gtccatccagtcgacaatactcataaccacgc

SEQ ID NO: 103 - DNA, 32 bp, synthetic construct
  source 1..32: mol_type = other DNA
gcttgtcgaagaggaagtggtcgtcgatgagc

SEQ ID NO: 104 - DNA, 32 bp, synthetic construct
  source 1..32: mol_type = other DNA
catccatctcgatcatcagtttgtcaatcttc

SEQ ID NO: 105 - DNA, 32 bp, synthetic construct
  source 1..32: mol_type = other DNA
cagagtggtgggctgatggctgcacgggctca

SEQ ID NO: 106 - DNA, 32 bp, synthetic construct
  source 1..32: mol_type = other DNA
ctcgagagtatacattgtgatacgtcaagtca

SEQ ID NO: 107 - DNA, 32 bp, synthetic construct
  source 1..32: mol_type = other DNA
tccaccttgtctatggcacatcaaactatgca

SEQ ID NO: 108 - DNA, 32 bp, synthetic construct
  source 1..32: mol_type = other DNA
cagcttgccggtggtgcagatgaacttcaggg

SEQ ID NO: 109 - DNA, 32 bp, synthetic construct
  source 1..32: mol_type = other DNA
cttgaagccctcggggaaggacagcttcaagt

SEQ ID NO: 110 - DNA, 32 bp, synthetic construct
  source 1..32: mol_type = other DNA
tctccgaacgtgtcacgtctttagcgactaaa

SEQ ID NO: 111 - DNA, 32 bp, synthetic construct
  source 1..32: mol_type = other DNA
ttggacaacctaacaaagcacagcccgccatg

SEQ ID NO: 112 - DNA, 32 bp, synthetic construct
  source 1..32: mol_type = other DNA
gccgcctgctaccttcatcaccaaattgcact

SEQ ID NO: 113 - DNA, 32 bp, synthetic construct
  source 1..32: mol_type = other DNA
caccattaccaacaataccgactccaacagcc

SEQ ID NO: 114 - DNA, 32 bp, synthetic construct
  source 1..32: mol_type = other DNA
attgtggatatttaattcgagttccatattgc

SEQ ID NO: 115 - DNA, 32 bp, synthetic construct
  source 1..32: mol_type = other DNA
gtccatctatcatatgtcgctgtgacattact

SEQ ID NO: 116 - DNA, 32 bp, synthetic construct
  source 1..32: mol_type = other DNA
ccgacaaaacaaatgacacggagagatgggac

SEQ ID NO: 117 - DNA, 32 bp, synthetic construct
  source 1..32: mol_type = other DNA
ttgatatgcacacctgcccgcagcccggtgcc

SEQ ID NO: 118 - DNA, 32 bp, synthetic construct
  source 1..32: mol_type = other DNA
ttcccgcgagagtcaaagatctccctggcatg

SEQ ID NO: 119 - DNA, 32 bp, synthetic construct
  source 1..32: mol_type = other DNA
ggcagaagcttccggcacagccggggcggagc

SEQ ID NO: 120 - DNA, 32 bp, synthetic construct
  source 1..32: mol_type = other DNA
gagatttctattctccattacgataatgaaca

SEQ ID NO: 121 - DNA, 32 bp, synthetic construct
  source 1..32: mol_type = other DNA
agcttctttaccaaactttcaattgctctttt

SEQ ID NO: 122 - DNA, 32 bp, synthetic construct
  source 1..32: mol_type = other DNA
tgaagcactgcacgccgtaggtcagggtggtc

SEQ ID NO: 123 - DNA, 32 bp, synthetic construct
  source 1..32: mol_type = other DNA
cgcacatgtccaccaccatgctaaccacttaa

SEQ ID NO: 124 - DNA, 32 bp, synthetic construct
  source 1..32: mol_type = other DNA
cctagcttcaccaccaaatcgttagcgctcct

SEQ ID NO: 125 - DNA, 32 bp, synthetic construct
  source 1..32: mol_type = other DNA
gaagatgcagcatctgaaaacctttaccccag

SEQ ID NO: 126 - DNA, 32 bp, synthetic construct
  source 1..32: mol_type = other DNA
gagcagagggtgaaggcctcctgagcgcaggg

SEQ ID NO: 127 - DNA, 32 bp, synthetic construct
  source 1..32: mol_type = other DNA
ctttgaatgaccagtcttaagatctttcttga

SEQ ID NO: 128 - DNA, 32 bp, synthetic construct
  source 1..32: mol_type = other DNA
aaaagttgagtaaggcccacagttcatgcagg

SEQ ID NO: 129 - DNA, 32 bp, synthetic construct
  source 1..32: mol_type = other DNA
cgagaacttctcatgcttgcccaggttgggca

SEQ ID NO: 130 - DNA, 32 bp, synthetic construct
  source 1..32: mol_type = other DNA
gacccccttctcaacggcaccagctttgcaga

SEQ ID NO: 131 - DNA, 32 bp, synthetic construct
  source 1..32: mol_type = other DNA
caaatacacatcatacttcccagcagagcggc

SEQ ID NO: 132 - DNA, 32 bp, synthetic construct
  source 1..32: mol_type = other DNA
aaattccacatacaactgcttcttcaagtctg

SEQ ID NO: 133 - DNA, 32 bp, synthetic construct
  source 1..32: mol_type = other DNA
tccaatgttctctgtatggtaacacatttact

SEQ ID NO: 134 - DNA, 32 bp, synthetic construct
  source 1..32: mol_type = other DNA
aggatgttgccgtcctccttgaagtcgatgcc

SEQ ID NO: 135 - DNA, 32 bp, synthetic construct
  source 1..32: mol_type = other DNA
gtcagtaaatgaacctttcctatcccacgtgt

SEQ ID NO: 136 - DNA, 32 bp, synthetic construct
  source 1..32: mol_type = other DNA
ccagtcatgatcattcctgatcacatattaag

SEQ ID NO: 137 - DNA, 30 bp, synthetic construct
  source 1..30: mol_type = other DNA
gccacttgaacactgcgacagaactggatc

SEQ ID NO: 138 - DNA, 30 bp, synthetic construct
  source 1..30: mol_type = other DNA
gccgcctgctaccttcatcaccaaattgca

SEQ ID NO: 139 - DNA, 30 bp, synthetic construct
  source 1..30: mol_type = other DNA
gcttgtcgaagaggaagtggtcgtcgatga

SEQ ID NO: 140 - DNA, 30 bp, synthetic construct
  source 1..30: mol_type = other DNA
tccaccttgtctatggcacatcaaactatg

SEQ ID NO: 141 - DNA, 30 bp, synthetic construct
  source 1..30: mol_type = other DNA
tctccgaacgtgtcacgtctttagcgacta

SEQ ID NO: 142 - DNA, 51 bp, synthetic construct
  source 1..51: mol_type = other DNA
gttctgtcgcagtgttcaagtcctgacccaacttgaacactgcgacagaac

SEQ ID NO: 143 - DNA, 51 bp, synthetic construct
  source 1..51: mol_type = other DNA
gtgatgaaggtagcaggcggccctgacccagccgcctgctaccttcatcac

SEQ ID NO: 144 - DNA, 51 bp, synthetic construct
  source 1..51: mol_type = other DNA
gaccacttcctcttcgacaagcctgacccacttgtcgaagaggaagtggtc

SEQ ID NO: 145 - DNA, 51 bp, synthetic construct
  source 1..51: mol_type = other DNA
gatgtgccatagacaaggtggcctgacccaccaccttgtctatggcacatc

SEQ ID NO: 146 - DNA, 51 bp, synthetic construct
  source 1..51: mol_type = other DNA
gctaaagacgtgacacgttcgcctgacccacgaacgtgtcacgtctttagc

SEQ ID NO: 147 - DNA, 24 bp, synthetic construct
  source 1..24: mol_type = other DNA
cagcttgccggtggtgcagatgaa

SEQ ID NO: 148 - DNA, 28 bp, synthetic construct
  source 1..28: mol_type = other DNA
cagcttgccggtggtgcagatgaacttc

SEQ ID NO: 149 - DNA, 32 bp, synthetic construct; identical to SEQ ID NO: 108
  source 1..32: mol_type = other DNA

SEQ ID NO: 150 - DNA, 36 bp, synthetic construct
  source 1..36: mol_type = other DNA
cagcttgccggtggtgcagatgaacttcagggtcag

SEQ ID NO: 151 - DNA, 40 bp, synthetic construct
  source 1..40: mol_type = other DNA
cagcttgccggtggtgcagatgaacttcagggtcagcttg

SEQ ID NO: 152 - DNA, 44 bp, synthetic construct
  source 1..44: mol_type = other DNA
cagcttgccggtggtgcagatgaacttcagggtcagcttgccgt

SEQ ID NO: 153 - DNA, 48 bp, synthetic construct
  source 1..48: mol_type = other DNA
cagcttgccggtggtgcagatgaacttcagggtcagcttgccgtaggt

SEQ ID NO: 154 - DNA, 32 bp, synthetic construct
  source 1..32: mol_type = other DNA
aaaagcaggtatccgcggccccgatgggcaaa

SEQ ID NO: 155 - DNA, 32 bp, synthetic construct; identical to SEQ ID NO: 98
  source 1..32: mol_type = other DNA

SEQ ID NO: 156 - DNA, 32 bp, synthetic construct; identical to SEQ ID NO: 113
  source 1..32: mol_type = other DNA

SEQ ID NO: 157 - DNA, 23 bp, synthetic construct
  source 1..23: mol_type = other DNA
  misc_feature 1: note = 5' Cy3 dye
gggcactccctgctggaagggaa

SEQ ID NO: 158 - DNA, 24 bp, synthetic construct
  source 1..24: mol_type = other DNA
  misc_feature 1: note = 5' Cy3 dye
aattgtgcaccttgactgtccaaa

SEQ ID NO: 159 - DNA, 22 bp, synthetic construct
  source 1..22: mol_type = other DNA
  misc_feature 1: note = 5' Cy3 dye
tctgagagtaggaccttattca

SEQ ID NO: 160 - DNA, 24 bp, synthetic construct
  source 1..24: mol_type = other DNA
  misc_feature 1: note = 5' Cy3 dye
tcagcacccctgctgtactgcaaa

SEQ ID NO: 161 - DNA, 4834 bp, synthetic construct
  source 1..4834: mol_type = other DNA
acatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcggatccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggattacaaagacgatgacgataagatggcgcctaagaagaaacgcaaagtgcggggcatgaagaaagaaaagattgatctgttttacggagccctgctgcacgacatcggaaaggtcatccagcgagcaaccggagagcggaagaaacacgcacttgtgggcgccgactggttcgacgagatcgccgacaaccaagtcatctcggatcagatccggtaccatatggccaactaccagtctgataagctcggcaacgatcacctggcttacatcacctacattgccgacaacatcgcctccggtgtcgaccgccggcaatccaacgaagagtcagacgaagatacctccgcaaagatctgggacacctacacgaaccaggccgacatctttaacgtgttcggagcgcagaccgataagcggtacttcaagcctaccgtgctgaatctcaagtcgaagcccaacttcgcgtccgccacttacgaaccctttagcaagggcgattacgctgccatcgccacccggattaagaacgaactggccgagttcgagttcaaccaagtccagattgactccctgctcaaccttttcgaggctactctctccttcgtgccgtcaagcaccaacactaaggaaatcgccgacatctccctggccgaccattcccgcttgactgctgccttcgctctggcgatctacgactacctggaggacaagggtcggcacaactacaaagaggacctgttcaccaaagtgtcagcgttctatgaagaagaagccttcctgctggcctccttcgacctgtcgggaatccaggactttatctacaacattaacatcgcaactaacggcgcggcgaagcagctgaaggcccggagcctctacctggactttatgtccgagtacatcgccgatagcctgctggacaagctgggactgaacagggctaacatgctttacgtcggcggcggacacgcctacttcgtcctggccaacaccgaaaagactgtggaaaccctggtgcagtttgagaaggatttcaaccagttcctgttggcaaacttccagacccgcctctatgtggcctttggctggggttccttcgcggccaaggacatcatgtccgagctgaatagccccgagtcctaccgccaagtgtaccaaaaggcttcgcgcatgatctccaaaaagaaaatctccagatacgactaccagacactgatgctcctgaatcgcggtggaaagtcctcagagagagagtgcgagatttgccactccgtggagaacctggtgtcctaccacgaccagaaagtctgtgacatttgccggggactgtaccagttctcgaaagaaattgcccatgaccacttcatcattaccgaaaatgaggggctgccgattggaccaaacgcgtgcttaaagggcgtggcattcgaaaagctgtcccaagaagcgttcagccgggtctacgtgaagaatgactataaggccggtaccgtgaaggctacgcatgtgttcgtgggggattaccagtgcgacgagatctacaactacgccgccctgagcaagaacgagaacggcctaggcatcaagagactggccgtggtccggctcgacgtggatgacttgggcgccgccttcatggccggtttcagccagcagggaaacggacaatactccactctgtcaagatcggccacattctcccggagcatgtcgctgttcttcaaagtgtacattaaccagttcgcctccgacaagaagctgagcattatctacgcgggcggcgatgacgtgttcgccattggatcgtggcaggatatcatcgcgttcactgtggaacttcgcgaaaacttcatcaagtggaccaacgggaagctcaccctctccgcggggatagggttgttcgccgacaagactcctattagcctgatggctcaccagaccggggaactggaagaggccgccaagggcaacgaaaaggactccatctcgctgttctcaagcgactacactttcaagtttgataggttcatcactaacgtgtacgacgacaaactggaacagattagatacttcttcaaccatcaagacgagaggggaaagaacttcatctataagcttattgagcttttgaggaaccacgaccgcatgaatatggcacgcctcgcctattacctcactcgcctggaagaactgacccgggagactgacagggacaagttcaagaccttcaagaacctgttctactcctggtacaccaacaagaacgataaggaccggaaggaagccgagctcgcgctcctgctgtacatctacgaaatcagaaaggattaacggcaataaaaagacagaataaaacgcacggtgttgggtcgtttgttcaagctc

SEQ ID NO: 162 - DNA, 4834 bp, synthetic construct
  source 1..4834: mol_type = other DNA
acatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcggatccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggattacaaagacgatgacgataagatggcgcctaagaagaaacgcaaagtgcggggcatgaagaaagaaaagattgatctgttttacggagccctgctggccgccatcggaaaggtcatccagcgagcaaccggagagcggaagaaacacgcacttgtgggcgccgactggttcgacgagatcgccgacaaccaagtcatctcggatcagatccggtaccatatggccaactaccagtctgataagctcggcaacgatcacctggcttacatcacctacattgccgacaacatcgcctccggtgtcgaccgccggcaatccaacgaagagtcagacgaagatacctccgcaaagatctgggacacctacacgaaccaggccgacatctttaacgtgttcggagcgcagaccgataagcggtacttcaagcctaccgtgctgaatctcaagtcgaagcccaacttcgcgtccgccacttacgaaccctttagcaagggcgattacgctgccatcgccacccggattaagaacgaactggccgagttcgagttcaaccaagtccagattgactccctgctcaaccttttcgaggctactctctccttcgtgccgtcaagcaccaacactaaggaaatcgccgacatctccctggccgaccattcccgcttgactgctgccttcgctctggcgatctacgactacctggaggacaagggtcggcacaactacaaagaggacctgttcaccaaagtgtcagcgttctatgaagaagaagccttcctgctggcctccttcgacctgtcgggaatccaggactttatctacaacattaacatcgcaactaacggcgcggcgaagcagctgaaggcccggagcctctacctggactttatgtccgagtacatcgccgatagcctgctggacaagctgggactgaacagggctaacatgctttacgtcggcggcggacacgcctacttcgtcctggccaacaccgaaaagactgtggaaaccctggtgcagtttgagaaggatttcaaccagttcctgttggcaaacttccagacccgcctctatgtggcctttggctggggttccttcgcggccaaggacatcatgtccgagctgaatagccccgagtcctaccgccaagtgtaccaaaaggcttcgcgcatgatctccaaaaagaaaatctccagatacgactaccagacactgatgctcctgaatcgcggtggaaagtcctcagagagagagtgcgagatttgccactccgtggagaacctggtgtcctaccacgaccagaaagtctgtgacatttgccggggactgtaccagttctcgaaagaaattgcccatgaccacttcatcattaccgaaaatgaggggctgccgattggaccaaacgcgtgcttaaagggcgtggcattcgaaaagctgtcccaagaagcgttcagccgggtctacgtgaagaatgactataaggccggtaccgtgaaggctacgcatgtgttcgtgggggattaccagtgcgacgagatctacaactacgccgccctgagcaagaacgagaacggcctaggcatcaagagactggccgtggtccggctcgacgtggatgacttgggcgccgccttcatggccggtttcagccagcagggaaacggacaatactccactctgtcaagatcggccacattctcccggagcatgtcgctgttcttcaaagtgtacattaaccagttcgcctccgacaagaagctgagcattatctacgcgggcggcgatgacgtgttcgccattggatcgtggcaggatatcatcgcgttcactgtggaacttcgcgaaaacttcatcaagtggaccaacgggaagctcaccctctccgcggggatagggttgttcgccgacaagactcctattagcctgatggctcaccagaccggggaactggaagaggccgccaagggcaacgaaaaggactccatctcgctgttctcaagcgactacactttcaagtttgataggttcatcactaacgtgtacgacgacaaactggaacagattagatacttcttcaaccatcaagacgagaggggaaagaacttcatctataagcttattgagcttttgaggaaccacgaccgcatgaatatggcacgcctcgcctattacctcactcgcctggaagaactgacccgggagactgacagggacaagttcaagaccttcaagaacctgttctactcctggtacaccaacaagaacgataaggaccggaaggaagccgagctcgcgctcctgctgtacatctacgaaatcagaaaggattaacggcaataaaaagacagaataaaacgcacggtgttgggtcgtttgttcaagctc

SEQ ID NO: 163 - DNA, 4834 bp, synthetic construct
  source 1..4834: mol_type = other DNA
acatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcggatccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggattacaaagacgatgacgataagatggcgcctaagaagaaacgcaaagtgcggggcatgaagaaagaaaagattgatctgttttacggagccctgctgcacgacatcggaaaggtcatccagcgagcaaccggagagcggaagaaacacgcacttgtgggcgccgactggttcgacgagatcgccgacaaccaagtcatctcggatcagatccggtaccatatggccaactaccagtctgataagctcggcaacgatcacctggcttacatcacctacattgccgacaacatcgcctccggtgtcgaccgccggcaatccaacgaagagtcagacgaagatacctccgcaaagatctgggacacctacacgaaccaggccgacatctttaacgtgttcggagcgcagaccgataagcggtacttcaagcctaccgtgctgaatctcaagtcgaagcccaacttcgcgtccgccacttacgaaccctttagcaagggcgattacgctgccatcgccacccggattaagaacgaactggccgagttcgagttcaaccaagtccagattgactccctgctcaaccttttcgaggctactctctccttcgtgccgtcaagcaccaacactaaggaaatcgccgacatctccctggccgaccattcccgcttgactgctgccttcgctctggcgatctacgactacctggaggacaagggtcggcacaactacaaagaggacctgttcaccaaagtgtcagcgttctatgaagaagaagccttcctgctggcctccttcgacctgtcgggaatccaggactttatctacaacattaacatcgcaactaacggcgcggcgaagcagctgaaggcccggagcctctacctggactttatgtccgagtacatcgccgatagcctgctggacaagctgggactgaacagggctaacatgctttacgtcggcggcggacacgcctacttcgtcctggccaacaccgaaaagactgtggaaaccctggtgcagtttgagaaggatttcaaccagttcctgttggcaaacttccagacccgcctctatgtggcctttggctggggttccttcgcggccaaggacatcatgtccgagctgaatagccccgagtcctaccgccaagtgtaccaaaaggcttcgcgcatgatctccaaaaagaaaatctccagatacgactaccagacactgatgctcctgaatcgcggtggaaagtcctcagagagagagtgcgagatttgccactccgtggagaacctggtgtcctaccacgaccagaaagtctgtgacatttgccggggactgtaccagttctcgaaagaaattgcccatgaccacttcatcattaccgaaaatgaggggctgccgattggaccaaacgcgtgcttaaagggcgtggcattcgaaaagctgtcccaagaagcgttcagccgggtctacgtgaagaatgactataaggccggtaccgtgaaggctacgcatgtgttcgtgggggattaccagtgcgacgagatctacaactacgccgccctgagcaagaacgagaacggcctaggcatcaagagactggccgtggtccggctcgacgtggatgacttgggcgccgccttcatggccggtttcagccagcagggaaacggacaatactccactctgtcaagatcggccacattctcccggagcatgtcgctgttcttcaaagtgtacattaaccagttcgcctccgacaagaagctgagcattatctacgcgggcggcgccgccgtgttcgccattggatcgtggcaggatatcatcgcgttcactgtggaacttcgcgaaaacttcatcaagtggaccaacgggaagctcaccctctccgcggggatagggttgttcgccgacaagactcctattagcctgatggctcaccagaccggggaactggaagaggccgccaagggcaacgaaaaggactccatctcgctgttctcaagcgactacactttcaagtttgataggttcatcactaacgtgtacgacgacaaactggaacagattagatacttcttcaaccatcaagacgagaggggaaagaacttcatctataagcttattgagcttttgaggaaccacgaccgcatgaatatggcacgcctcgcctattacctcactcgcctggaagaactgacccgggagactgacagggacaagttcaagaccttcaagaacctgttctactcctggtacaccaacaagaacgataaggaccggaaggaagccgagctcgcgctcctgctgtacatctacgaaatcagaaaggattaacggcaataaaaagacagaataaaacgcacggtgttgggtcgtttgttcaagctc

SEQ ID NO: 164 - DNA, 2938 bp, synthetic construct
  source 1..2938: mol_type = other DNA
acatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcggatccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggattacaaagacgatgacgataagatggcgcctaagaagaaacgcaaagtgcggggcatgaccatcctgaccgacgagaactacgtggacatcgccgagaaagccatcctgaagctggaaagaaacaccagaaatagaaagaaccctgatgccttcttcctgaccacatctaagctgcggaacctgctgagcctgacaagcaccctgttcgacgagagcaaggtgaaggaatacgacgccctgctggacagaatcgcttatctgagagtgcagttcgtgtaccaggccggcagagagatcgccgtgaaagatctgatcgagaaggcccagatcctggaagctctgaaagagatcaaggaccgggaaaccctgcagagattctgcagatacatggaagccctggtggcctacttcaagttctacggcggcaaggactgacggcaataaaaagacagaataaaacgcacggtgttgggtcgtttgttcaagctc

SEQ ID NO: 165 - DNA, 3220 bp, synthetic construct
  source 1..3220: mol_type = other DNA
acatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcggatccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggattacaaagacgatgacgataagatggcgcctaagaagaaacgcaaagtgcggggcatgaccttcgccaagatcaaattcagcgcccagatccggctggaaaccggcctgcacatcggaggatctgatgcctttgccgctatcggcgccatcgacagccctgtgatcaaggaccccatcaccaacctgcctatcatccccggctctagcctgaagggcaagatgagaacactgctggccaaggtgtacaacgaaaaggtggccgagaagcctagcgacgacagcgacatcctgagcagactgttcggaaatagcaaggataagcggttcaagatgggcagactgatcttccgggacgccttcctgagcaacgccgacgagctggattctctgggcgtgcggagctacaccgaggtgaagttcgagaacaccatcgatagaatcaccgccgaggccaatcctagacagatcgagagagccattcggaactcaacattcgacttcgagctgatctacgagatcactgatgagaatgagaaccaggtcgaggaagatttcaaggtgatcagagacggcctgaagctgctggaactggactacctgggcggaagcggctccagaggctacggcaaagtggcttttgagaacctgaaagccaccacagtgttcggcaactacgacgtgaaaaccctgaacgagctgctgaccgccgaagtgtgacggcaataaaaagacagaataaaacgcacggtgttgggtcgtttgttcaagctc

SEQ ID NO: 166 - DNA, 3220 bp, synthetic construct
  source 1..3220: mol_type = other DNA
acatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcggatccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggattacaaagacgatgacgataagatggcgcctaagaagaaacgcaaagtgcggggcatgaccttcgccaagatcaaattcagcgcccagatccggctggaaaccggcctgcacatcggaggatctgatgcctttgccgctatcggcgccatcgccagccctgtgatcaaggaccccatcaccaacctgcctatcatccccggctctagcctgaagggcaagatgagaacactgctggccaaggtgtacaacgaaaaggtggccgagaagcctagcgacgacagcgacatcctgagcagactgttcggaaatagcaaggataagcggttcaagatgggcagactgatcttccgggacgccttcctgagcaacgccgacgagctggattctctgggcgtgcggagctacaccgaggtgaagttcgagaacaccatcgatagaatcaccgccgaggccaatcctagacagatcgagagagccattcggaactcaacattcgacttcgagctgatctacgagatcactgatgagaatgagaaccaggtcgaggaagatttcaaggtgatcagagacggcctgaagctgctggaactggactacctgggcggaagcggctccagaggctacggcaaagtggcttttgagaacctgaaagccaccacagtgttcggcaactacgacgtgaaaaccctgaacgagctgctgaccgccgaagtgtgacggcaataaaaagacagaataaaacgcacggtgttgggtcgtttgttcaagctc

SEQ ID NO: 167 - DNA, 3457 bp, synthetic construct
  source 1..3457: mol_type = other DNA
acatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcggatccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggattacaaagacgatgacgataagatggcgcctaagaagaaacgcaaagtgcggggcatgacttacaagctctacattatgacctttcaaaacgcccacttcggttccggcactctggactcatcgaagctgaccttctccgcggatagaatcttctcggcactcgtgctcgaggctctgaagatgggaaagctcgacgccttcttggccgaggccaaccaggataagttcactctgaccgacgcgttcccattccaattcggtcctttcctgccgaaaccgattggttaccccaagcacgaccagatcgaccagtctgtggacgtgaaggaagtccgccgccaagcgaagctgtccaaaaagctccagttcctggctctggaaaacgtcgacgactacctgaacggagagctgtttgagaatgaggaacacgccgtgatcgacacagtgaccaagaaccagccccataaagatgataatctgtaccaagtggccaccactcggttctcgaacgacacctccctttacgtgatcgccaacgaatccgatctgctgaacgaactgatgagcagccttcagtactccgggctgggcggcaaaaggtcctcaggattcggcagatttgagctggacatccagaacattcccttggaactgtccgaccggctgacgaagaaccacagcgacaaggtcatgtcacttaccaccgccctcccggtggacgctgatctcgaggaagcgatggaagatggccattacctgttgaccaagtcgtccggattcgcattctcccacgccaccaacgaaaactatcggaagcaggacctgtacaagttcgcctccgggagcaccttcagcaagactttcgagggacagatcgtggacgtgcgccctctcgatttccctcacgccgtgctgaactacgccaagccgctgttctttaagctcgaagtctaacggcaataaaaagacagaataaaacgcacggtgttgggtcgtttgttcaagctc

SEQ ID NO: 168 - DNA, 3631 bp, synthetic construct
  source 1..3631: mol_type = other DNA
acatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcggatccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggattacaaagacgatgacgataagatggcgcctaagaagaaacgcaaagtgcggggcatgaaaaatgactaccggaccttcaagctgagcctgctgaccctggctcctatccacatcggcaacggcgagaagtacaccagcagagaattcatctacgagaacaagaagttctacttccccgacatgggcaagttctacaacaagatggtggaaaagagactggccgagaagttcgaggccttcctgatccagaccagacccaacgccagaaacaaccggctgatttcttttctgaacgacaacagaatcgccgaaagatcttttggcggctacagcatcagtgaaaccggcctggaatctgataagaaccctaacagcgccggagctatcaacgaggtgaacaaattcatccgggacgccttcggaaatccttacatcccaggcagcagcctgaagggcgccatccgcaccatcctgatgaacaccacacctaagtggaacaacgagaacgccgtgaacgacttcggcagattcccaaaggaaaacaagaacctgatcccttggggacctaagaaaggcaaggaatacgacgacctgttcaacgccatcagagtgtccgacagcaagcccttcgacaacaaaagcctgatcctcgtgcagaagtgggactacagcgccaaaaccaacaaggccaagcctctgcctctgtacagagagtctatcagccctctgaccaagatcgagttcgagataacaacaaccactgatgaggccggcagactgatcgaggaactgggaaagcgggcccaggccttttataaggactacaaggcctttttcctgtctgaattccctgatgataagatccaggctaatctgcaataccccatctacctgggcgccggcagcggcgcttggacaaagaccctgtttaagcaggccgacggcatcctgcagcggagatactccagaatgaaaaccaagatggtcaagaagggcgtgctgaagctgacaaaggcccctctgaaaacagtgaagatccccagcggcaaccacagcctggtgaagaatcacgagagcttctacgagatgggcaaagccaacttcatgatcaaggaaatcgacaagtgacggcaataaaaagacagaataaaacgcacggtgttgggtcgtttgttcaagctc

SEQ ID NO: 169 - DNA, 3289 bp, synthetic construct
  source 1..3289: mol_type = other DNA
acatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcggatccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggattacaaagacgatgacgataagatggcgcctaagaagaaacgcaaagtgcggggcatgaaaaagctcgtgttcacctttaagcggatcgaccaccctgctcaggacctggccgtgaaattccacggcttcctgatggaacagctggatagcgactacgtggactacctgcaccagcagcagaccaacccctacgccacaaaggtgatccagggcaaagagaacacccagtgggtcgtgcatctgctgacagacgacatcgaggacaaggtgttcatgaccctgctgcagatcaaggaagtgtccctgaacgacctgcctaagttgtctgtggaaaaggtggaaatccaggagctgggcgctgataagctgctcgagatcttcaacagcgaggaaaaccagacctacttcagcatcatcttcgagacacctacaggctttaaaagccagggcagctacgtgatcttccccagcatgcggctgatctttcagagcctgatgcagaagtacggcagactggtggaaaaccagcctgagatcgaggaagataccctggactacctgagcgagcacagcaccatcaccaattacagactggaaacaagctacttcagagtgcatagacagagaatccccgccttccggggcaagctgaccttcaaggtgcagggagcccagacactgaaggcctacgtgaagatgctgctgaccttcggcgagtacagcggcctgggcatgaaaaccagcctgggaatgggcggcatcaagctggaagaaagaaaggactgacggcaataaaaagacagaataaaacgcacggtgttgggtcgtttgttcaagctc

SEQ ID NO: 170 - DNA, 2420 bp, synthetic construct
  source 1..2420: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgatataaacctaattacctcgagaggggacggaaacccgtcttcgatgaagcgattcagaagacttgatataaacctaattacctcgagaggggacttttttacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 171 - DNA, 10797 bp, synthetic construct
  source 1..10797: mol_type = other DNA
ctcgagtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagggatccgccgccaccatggattacaaagacgatgacgataagatggcgcctaagaagaaacgcaaagtgcggggcatgaaaaatgactaccggaccttcaagctgagcctgctgaccctggctcctatccacatcggcaacggcgagaagtacaccagcagagaattcatctacgagaacaagaagttctacttccccgacatgggcaagttctacaacaagatggtggaaaagagactggccgagaagttcgaggccttcctgatccagaccagacccaacgccagaaacaaccggctgatttcttttctgaacgacaacagaatcgccgaaagatcttttggcggctacagcatcagtgaaaccggcctggaatctgataagaaccctaacagcgccggagctatcaacgaggtgaacaaattcatccgggacgccttcggaaatccttacatcccaggcagcagcctgaagggcgccatccgcaccatcctgatgaacaccacacctaagtggaacaacgagaacgccgtgaacgacttcggcagattcccaaaggaaaacaagaacctgatcccttggggacctaagaaaggcaaggaatacgacgacctgttcaacgccatcagagtgtccgacagcaagcccttcgacaacaaaagcctgatcctcgtgcagaagtgggactacagcgccaaaaccaacaaggccaagcctctgcctctgtacagagagtctatcagccctctgaccaagatcgagttcgagataacaacaaccactgatgaggccggcagactgatcgaggaactgggaaagcgggcccaggccttttataaggactacaaggcctttttcctgtctgaattccctgatgataagatccaggctaatctgcaataccccatctacctgggcgccggcagcggcgcttggacaaagaccctgtttaagcaggccgacggcatcctgcagcggagatactccagaatgaaaaccaagatggtcaagaagggcgtgctgaagctgacaaaggcccctctgaaaacagtgaagatccccagcggcaaccacagcctggtgaagaatcacgagagcttctacgagatgggcaaagccaacttcatgatcaaggaaatcgacaaggaaggaaggggctccctcctcacttgtggagatgtcgaagaaaatcctggacctgattacaaagacgatgacgataagatggcgcctaagaagaaacgcaaagtgcggggcatgacttacaagctctacattatgacctttcaaaacgcccacttcggttccggcactctggactcatcgaagctgaccttctccgcggatagaatcttctcggcactcgtgctcgaggctctgaagatgggaaagctcgacgccttcttggccgaggccaaccaggataagttcactctgaccgacgcgttcccattccaattcggtcctttcctgccgaaaccgattggttaccccaagcacgaccagatcgaccagtctgtggacgtgaaggaagtccgccgccaagcgaagctgtccaaaaagctccagttcctggctctggaaaacgtcgacgactacctgaacggagagctgtttgagaatgaggaacacgccgtgatcgacacagtgaccaagaaccagccccataaagatgataatctgtaccaagtggccaccactcggttctcgaacgacacctccctttacgtgatcgccaacgaatccgatctgctgaacgaactgatgagcagccttcagtactccgggctgggcggcaaaaggtcctcaggattcggcagatttgagctggacatccagaacattcccttggaactgtccgaccggctgacgaagaaccacagcgacaaggtcatgtcacttaccaccgccctcccggtggacgctgatctcgaggaagcgatggaagatggccattacctgttgaccaagtcgtccggattcgcattctcccacgccaccaacgaaaactatcggaagcaggacctgtacaagttcgcctccgggagcaccttcagcaagactttcgagggacagatcgtggacgtgcgccctctcgatttccctcacgccgtgctgaactacgccaagccgctgttctttaagctcgaagtcgagggcagaggaagtctgctaacatgcggtgacgtcgaggagaatcctggacctgattacaaagacgatgacgataagatggcgcctaagaagaaacgcaaagtgcggggcatgaccttcgccaagatcaaattcagcgcccagatccggctggaaaccggcctgcacatcggaggatctgatgcctttgccgctatcggcgccatcgacagccctgtgatcaaggaccccatcaccaacctgcctatcatccccggctctagcctgaagggcaagatgagaacactgctggccaaggtgtacaacgaaaaggtggccgagaagcctagcgacgacagcgacatcctgagcagactgttcggaaatagcaaggataagcggttcaagatgggcagactgatcttccgggacgccttcctgagcaacgccgacgagctggattctctgggcgtgcggagctacaccgaggtgaagttcgagaacaccatcgatagaatcaccgccgaggccaatcctagacagatcgagagagccattcggaactcaacattcgacttcgagctgatctacgagatcactgatgagaatgagaaccaggtcgaggaagatttcaaggtgatcagagacggcctgaagctgctggaactggactacctgggcggaagcggctccagaggctacggcaaagtggcttttgagaacctgaaagccaccacagtgttcggcaactacgacgtgaaaaccctgaacgagctgctgaccgccgaagtggaggggcgggggtctttgttgacttgcggggatgttgaggagaacccagggccagattacaaagacgatgacgataagatggcgcctaagaagaaacgcaaagtgcggggcatgaccatcctgaccgacgagaactacgtggacatcgccgagaaagccatcctgaagctggaaagaaacaccagaaatagaaagaaccctgatgccttcttcctgaccacatctaagctgcggaacctgctgagcctgacaagcaccctgttcgacgagagcaaggtgaaggaatacgacgccctgctggacagaatcgcttatctgagagtgcagttcgtgtaccaggccggcagagagatcgccgtgaaagatctgatcgagaaggcccagatcctggaagctctgaaagagatcaaggaccgggaaaccctgcagagattctgcagatacatggaagccctggtggcctacttcaagttctacggcggcaaggactgagctagcgcggccgcatcgataagcttgtcgacgatatctccagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgccccgagcttcctcgctcactgactcgctgcgctcggtcgttcggctgcggcgagcggtatcagctcactcaaaggcggtaatacggttatccacagaatcaggggataacgcaggaaagaaactagtgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgatataaacctaattacctcgagaggggacggaaacccgtcttcgatgaagcgattcagaagacttgatataaacctaattacctcgagaggggacttttttacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagattaatcctttctgatttcgtagatgtacagcaggagcgcgagctcggcttccttccggtccttatcgttcttgttggtgtaccaggagtagaacaggttcttgaaggtcttgaacttgtccctgtcagtctcccgggtcagttcttccaggcgagtgaggtaataggcgaggcgtgccatattcatgcggtcgtggttcctcaaaagctcaataagcttatagatgaagttctttcccctctcgtcttgatggttgaagaagtatctaatctgttccagtttgtcgtcgtacacgttagtgatgaacctatcaaacttgaaagtgtagtcgcttgagaacagcgagatggagtccttttcgttgcccttggcggcctcttccagttccccggtctggtgagccatcaggctaataggagtcttgtcggcgaacaaccctatccccgcggagagggtgagcttcccgttggtccacttgatgaagttttcgcgaagttccacagtgaacgcgatgatatcctgccacgatccaatggcgaacacgtcatcgccgcccgcgtagataatgctcagcttcttgtcggaggcgaactggttaatgtacactttgaagaacagcgacatgctccgggagaatgtggccgatcttgacagagtggagtattgtccgtttccctgctggctgaaaccggccatgaaggcggcgcccaagtcatccacgtcgagccggaccacggccagtctcttgatgcctaggccgttctcgttcttgctcagggcggcgtagttgtagatctcgtcgcactggtaatcccccacgaacacatgcgtagccttcacggtaccggccttatagtcattcttcacgtagacccggctgaacgcttcttgggacagcttttcgaatgccacgccctttaagcacgcgtttggtccaatcggcagcccctcattttcggtaatgatgaagtggtcatgggcaatttctttcgagaactggtacagtccccggcaaatgtcacagactttctggtcgtggtaggacaccaggttctccacggagtggcaaatctcgcactctctctctgaggactttccaccgcgattcaggagcatcagtgtctggtagtcgtatctggagattttctttttggagatcatgcgcgaagccttttggtacacttggcggtaggactcggggctattcagctcggacatgatgtccttggccgcgaaggaaccccagccaaaggccacatagaggcgggtctggaagtttgccaacaggaactggttgaaatccttctcaaactgcaccagggtttccacagtcttttcggtgttggccaggacgaagtaggcgtgtccgccgccgacgtaaagcatgttagccctgttcagtcccagcttgtccagcaggctatcggcgatgtactcggacataaagtccaggtagaggctccgggccttcagctgcttcgccgcgccgttagttgcgatgttaatgttgtagataaagtcctggattcccgacaggtcgaaggaggccagcaggaaggcttcttcttcatagaacgctgacactttggtgaacaggtcctctttgtagttgtgccgacccttgtcctccaggtagtcgtagatcgccagagcgaaggcagcagtcaagcgggaatggtcggccagggagatgtcggcgatttccttagtgttggtgcttgacggcacgaaggagagagtagcctcgaaaaggttgagcagggagtcaatctggacttggttgaactcgaactcggccagttcgttcttaatccgggtggcgatggcagcgtaatcgcccttgctaaagggttcgtaagtggcggacgcgaagttgggcttcgacttgagattcagcacggtaggcttgaagtaccgcttatcggtctgcgctccgaacacgttaaagatgtcggcctggttcgtgtaggtgtcccagatctttgcggaggtatcttcgtctgactcttcgttggattgccggcggtcgacaccggaggcgatgttgtcggcaatgtaggtgatgtaagccaggtgatcgttgccgagcttatcagactggtagttggccatatggtaccggatctgatccgagatgacttggttgtcggcgatctcgtcgaaccagtcggcgcccacaagtgcgtgtttcttccgctctccggttgctcgctggatgacctttccgatgtcgtgcagcagggctccgtaaaacagatcaatcttttctttcttcatgccccgcactttgcgtttcttcttaggcgccatcttatcgtcatcgtctttgtaatcgccggaatggtgatggtgatggtgtggtcctggattttcttcaacatctccacaagttagcaaacttcctcttccttcgtcctttctttcttccagcttgatgccgcccattcccaggctggttttcatgcccaggccgctgtactcgccgaaggtcagcagcatcttcacgtaggccttcagtgtctgggctccctgcaccttgaaggtcagcttgccccggaaggcggggattctctgtctatgcactctgaagtagcttgtttccagtctgtaattggtgatggtgctgtgctcgctcaggtagtccagggtatcttcctcgatctcaggctggttttccaccagtctgccgtacttctgcatcaggctctgaaagatcagccgcatgctggggaagatcacgtagctgccctggcttttaaagcctgtaggtgtctcgaagatgatgctgaagtaggtctggttttcctcgctgttgaagatctcgagcagcttatcagcgcccagctcctggatttccaccttttccacagacaacttaggcaggtcgttcagggacacttccttgatctgcagcagggtcatgaacaccttgtcctcgatgtcgtctgtcagcagatgcacgacccactgggtgttctctttgccctggatcacctttgtggcgtaggggttggtctgctgctggtgcaggtagtccacgtagtcgctatccagctgttccatcaggaagccgtggaatttcacggccaggtcctgagcagggtggtcgatccgcttaaaggtgaacacgagctttttcatgccccgcactttgcgtttcttcttaggcgccatcttatcgtcatcgtctttgtaatcggggccggggttctcctccacgtcgccgcaggtcagcaggctgcctctgccctccttgtacagctcgtccatgccgccggtggagtggcggccctcggcgcgttcgtactgttccacgatggtgtagtcctcgttgtgggaggtgatgtccaacttgatgttgacgttgtaggcgccgggcagctgcacgggcttcttggccttgtaggtggtcttgacctcagcgtcgtagtggccgccgtccttcagcttcagcctctgcttgatctcgcccttcagggcgccgtcctcggggtacatccgctcggaggaggcctcccagcccatggttttcttctgcattacggggccgtcggaggggaagttggtgccgcgcagcttcaccttgtagatgaactcgccgtcctgcagggaggagtcctgggtcacggtcaccacgccgccgtcctcgaagttcatcacgcgctcccacttgaagccctcggggaaggacagcttcaagtagtcggggatgtcggcggggtgcttcacgtaggccttggagccgtacatgaactgaggggacaggatgtcccaggcgaagggcagggggccacccttggtcaccttcagcttggcggtctgggtgccctcgtaggggcggccctcgccctcgccctcgatctcgaactcgtggccgttcacggagccctccatgtgcaccttgaagcgcatgaactccttgatgatggccatgttatcctcctcgcccttgctcaccatggtggcggcaccggtgaattctccaggcgatctgacggttcactaaacgagctctgcttatataggcctcccaccgtacacgccacctcgacata

SEQ ID NO: 172 - DNA, 11591 bp, synthetic construct
  source 1..11591: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatgcaccatcaccatcaccattccggcgattacaaagacgatgacgataagatggcgcctaagaagaaacgcaaagtgcggggcatgaagaaagaaaagattgatctgttttacggagccctgctgcacgacatcggaaaggtcatccagcgagcaaccggagagcggaagaaacacgcacttgtgggcgccgactggttcgacgagatcgccgacaaccaagtcatctcggatcagatccggtaccatatggccaactaccagtctgataagctcggcaacgatcacctggcttacatcacctacattgccgacaacatcgcctccggtgtcgaccgccggcaatccaacgaagagtcagacgaagatacctccgcaaagatctgggacacctacacgaaccaggccgacatctttaacgtgttcggagcgcagaccgataagcggtacttcaagcctaccgtgctgaatctcaagtcgaagcccaacttcgcgtccgccacttacgaaccctttagcaagggcgattacgctgccatcgccacccggattaagaacgaactggccgagttcgagttcaaccaagtccagattgactccctgctcaaccttttcgaggctactctctccttcgtgccgtcaagcaccaacactaaggaaatcgccgacatctccctggccgaccattcccgcttgactgctgccttcgctctggcgatctacgactacctggaggacaagggtcggcacaactacaaagaggacctgttcaccaaagtgtcagcgttctatgaagaagaagccttcctgctggcctccttcgacctgtcgggaatccaggactttatctacaacattaacatcgcaactaacggcgcggcgaagcagctgaaggcccggagcctctacctggactttatgtccgagtacatcgccgatagcctgctggacaagctgggactgaacagggctaacatgctttacgtcggcggcggacacgcctacttcgtcctggccaacaccgaaaagactgtggaaaccctggtgcagtttgagaaggatttcaaccagttcctgttggcaaacttccagacccgcctctatgtggcctttggctggggttccttcgcggccaaggacatcatgtccgagctgaatagccccgagtcctaccgccaagtgtaccaaaaggcttcgcgcatgatctccaaaaagaaaatctccagatacgactaccagacactgatgctcctgaatcgcggtggaaagtcctcagagagagagtgcgagatttgccactccgtggagaacctggtgtcctaccacgaccagaaagtctgtgacatttgccggggactgtaccagttctcgaaagaaattgcccatgaccacttcatcattaccgaaaatgaggggctgccgattggaccaaacgcgtgcttaaagggcgtggcattcgaaaagctgtcccaagaagcgttcagccgggtctacgtgaagaatgactataaggccggtaccgtgaaggctacgcatgtgttcgtgggggattaccagtgcgacgagatctacaactacgccgccctgagcaagaacgagaacggcctaggcatcaagagactggccgtggtccggctcgacgtggatgacttgggcgccgccttcatggccggtttcagccagcagggaaacggacaatactccactctgtcaagatcggccacattctcccggagcatgtcgctgttcttcaaagtgtacattaaccagttcgcctccgacaagaagctgagcattatctacgcgggcggcgatgacgtgttcgccattggatcgtggcaggatatcatcgcgttcactgtggaacttcgcgaaaacttcatcaagtggaccaacgggaagctcaccctctccgcggggatagggttgttcgccgacaagactcctattagcctgatggctcaccagaccggggaactggaagaggccgccaagggcaacgaaaaggactccatctcgctgttctcaagcgactacactttcaagtttgataggttcatcactaacgtgtacgacgacaaactggaacagattagatacttcttcaaccatcaagacgagaggggaaagaacttcatctataagcttattgagcttttgaggaaccacgaccgcatgaatatggcacgcctcgcctattacctcactcgcctggaagaactgacccgggagactgacagggacaagttcaagaccttcaagaacctgttctactcctggtacaccaacaagaacgataaggaccggaaggaagccgagctcgcgctcctgctgtacatctacgaaatcagaaaggattaacggcaataaaaagacagaataaaacgcacggtgttgggtcgtttgttcgcacacattagctagccgtcagcacacattgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggattacaaagacgatgacgataagatggcgcctaagaagaaacgcaaagtgcggggcatgaccatcctgaccgacgagaactacgtggacatcgccgagaaagccatcctgaagctggaaagaaacaccagaaatagaaagaaccctgatgccttcttcctgaccacatctaagctgcggaacctgctgagcctgacaagcaccctgttcgacgagagcaaggtgaaggaatacgacgccctgctggacagaatcgcttatctgagagtgcagttcgtgtaccaggccggcagagagatcgccgtgaaagatctgatcgagaaggcccagatcctggaagctctgaaagagatcaaggaccgggaaaccctgcagagattctgcagatacatggaagccctggtggcctacttcaagttctacggcggcaaggactgacggcaataaaaagacagaataaaacgcacggtgttgggtcgtttgttcagttcttttgccttactttcaatgcatgcggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggattacaaagacgatgacgataagatggcgcctaagaagaaacgcaaagtgcggggcatgaccttcgccaagatcaaattcagcgcccagatccggctggaaaccggcctgcacatcggaggatctgatgcctttgccgctatcggcgccatcgacagccctgtgatcaaggaccccatcaccaacctgcctatcatccccggctctagcctgaagggcaagatgagaacactgctggccaaggtgtacaacgaaaaggtggccgagaagcctagcgacgacagcgacatcctgagcagactgttcggaaatagcaaggataagcggttcaagatgggcagactgatcttccgggacgccttcctgagcaacgccgacgagctggattctctgggcgtgcggagctacaccgaggtgaagttcgagaacaccatcgatagaatcaccgccgaggccaatcctagacagatcgagagagccattcggaactcaacattcgacttcgagctgatctacgagatcactgatgagaatgagaaccaggtcgaggaagatttcaaggtgatcagagacggcctgaagctgctggaactggactacctgggcggaagcggctccagaggctacggcaaagtggcttttgagaacctgaaagccaccacagtgttcggcaactacgacgtgaaaaccctgaacgagctgctgaccgccgaagtgtgacggcaataaaaagacagaataaaacgcacggtgttgggtcgtttgttctggattgcgagaatggactagtagcaaactgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggattacaaagacgatgacgataagatggcgcctaagaagaaacgcaaagtgcggggcatgacttacaagctctacattatgacctttcaaaacgcccacttcggttccggcactctggactcatcgaagctgaccttctccgcggatagaatcttctcggcactcgtgctcgaggctctgaagatgggaaagctcgacgccttcttggccgaggccaaccaggataagttcactctgaccgacgcgttcccattccaattcggtcctttcctgccgaaaccgattggttaccccaagcacgaccagatcgaccagtctgtggacgtgaaggaagtccgccgccaagcgaagctgtccaaaaagctccagttcctggctctggaaaacgtcgacgactacctgaacggagagctgtttgagaatgaggaacacgccgtgatcgacacagtgaccaagaaccagccccataaagatgataatctgtaccaagtggccaccactcggttctcgaacgacacctccctttacgtgatcgccaacgaatccgatctgctgaacgaactgatgagcagccttcagtactccgggctgggcggcaaaaggtcctcaggattcggcagatttgagctggacatccagaacattcccttggaactgtccgaccggctgacgaagaaccacagcgacaaggtcatgtcacttaccaccgccctcccggtggacgctgatctcgaggaagcgatggaagatggccattacctgttgaccaagtcgtccggattcgcattctcccacgccaccaacgaaaactatcggaagcaggacctgtacaagttcgcctccgggagcaccttcagcaagactttcgagggacagatcgtggacgtgcgccctctcgatttccctcacgccgtgctgaactacgccaagccgctgttctttaagctcgaagtctaacggcaataaaaagacagaataaaacgcacggtgttgggtcgtttgttcgcacattcaaaaacaggcaattggacaagcgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggattacaaagacgatgacgataagatggcgcctaagaagaaacgcaaagtgcggggcatgaaaaatgactaccggaccttcaagctgagcctgctgaccctggctcctatccacatcggcaacggcgagaagtacaccagcagagaattcatctacgagaacaagaagttctacttccccgacatgggcaagttctacaacaagatggtggaaaagagactggccgagaagttcgaggccttcctgatccagaccagacccaacgccagaaacaaccggctgatttcttttctgaacgacaacagaatcgccgaaagatcttttggcggctacagcatcagtgaaaccggcctggaatctgataagaaccctaacagcgccggagctatcaacgaggtgaacaaattcatccgggacgccttcggaaatccttacatcccaggcagcagcctgaagggcgccatccgcaccatcctgatgaacaccacacctaagtggaacaacgagaacgccgtgaacgacttcggcagattcccaaaggaaaacaagaacctgatcccttggggacctaagaaaggcaaggaatacgacgacctgttcaacgccatcagagtgtccgacagcaagcccttcgacaacaaaagcctgatcctcgtgcagaagtgggactacagcgccaaaaccaacaaggccaagcctctgcctctgtacagagagtctatcagccctctgaccaagatcgagttcgagataacaacaaccactgatgaggccggcagactgatcgaggaactgggaaagcgggcccaggccttttataaggactacaaggcctttttcctgtctgaattccctgatgataagatccaggctaatctgcaataccccatctacctgggcgccggcagcggcgcttggacaaagaccctgtttaagcaggccgacggcatcctgcagcggagatactccagaatgaaaaccaagatggtcaagaagggcgtgctgaagctgacaaaggcccctctgaaaacagtgaagatccccagcggcaaccacagcctggtgaagaatcacgagagcttctacgagatgggcaaagccaacttcatgatcaaggaaatcgacaagtgacggcaataaaaagacagaataaaacgcacggtgttgggtcgtttgttcatgtgtgccggccggagaataaaagtctaagtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggattacaaagacgatgacgataagatggcgcctaagaagaaacgcaaagtgcggggcatgaaaaagctcgtgttcacctttaagcggatcgaccaccctgctcaggacctggccgtgaaattccacggcttcctgatggaacagctggatagcgactacgtggactacctgcaccagcagcagaccaacccctacgccacaaaggtgatccagggcaaagagaacacccagtgggtcgtgcatctgctgacagacgacatcgaggacaaggtgttcatgaccctgctgcagatcaaggaagtgtccctgaacgacctgcctaagttgtctgtggaaaaggtggaaatccaggagctgggcgctgataagctgctcgagatcttcaacagcgaggaaaaccagacctacttcagcatcatcttcgagacacctacaggctttaaaagccagggcagctacgtgatcttccccagcatgcggctgatctttcagagcctgatgcagaagtacggcagactggtggaaaaccagcctgagatcgaggaagataccctggactacctgagcgagcacagcaccatcaccaattacagactggaaacaagctacttcagagtgcatagacagagaatccccgccttccggggcaagctgaccttcaaggtgcagggagcccagacactgaaggcctacgtgaagatgctgctgaccttcggcgagtacagcggcctgggcatgaaaaccagcctgggaatgggcggcatcaagctggaagaaagaaaggactgacggcaataaaaagacagaataaaacgcacggtgttgggtcgtttgttcgtgcgatagagggatcccgcattgaattatgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaaaaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaacggcaataaaaagacagaataaaacgcacggtgttgggtcgtttgttcgagcagattgtactgagagtgcaccggttggagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgatataaacctaattacctcgagaggggacggaaacccgtcttcgatgaagcgattcagaagacttgatataaacctaattacctcgagaggggacttttttacatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttcttaagc

SEQ ID NO: 173 - DNA, 11600 bp, synthetic construct
  source 1..11600: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatgcaccatcaccatcaccattccggcgattacaaagacgatgacgataagatggcgcctaagaagaaacgcaaagtgcggggcatgaagaaagaaaagattgatctgttttacggagccctgctgcacgacatcggaaaggtcatccagcgagcaaccggagagcggaagaaacacgcacttgtgggcgccgactggttcgacgagatcgccgacaaccaagtcatctcggatcagatccggtaccatatggccaactaccagtctgataagctcggcaacgatcacctggcttacatcacctacattgccgacaacatcgcctccggtgtcgaccgccggcaatccaacgaagagtcagacgaagatacctccgcaaagatctgggacacctacacgaaccaggccgacatctttaacgtgttcggagcgcagaccgataagcggtacttcaagcctaccgtgctgaatctcaagtcgaagcccaacttcgcgtccgccacttacgaaccctttagcaagggcgattacgctgccatcgccacccggattaagaacgaactggccgagttcgagttcaaccaagtccagattgactccctgctcaaccttttcgaggctactctctccttcgtgccgtcaagcaccaacactaaggaaatcgccgacatctccctggccgaccattcccgcttgactgctgccttcgctctggcgatctacgactacctggaggacaagggtcggcacaactacaaagaggacctgttcaccaaagtgtcagcgttctatgaagaagaagccttcctgctggcctccttcgacctgtcgggaatccaggactttatctacaacattaacatcgcaactaacggcgcggcgaagcagctgaaggcccggagcctctacctggactttatgtccgagtacatcgccgatagcctgctggacaagctgggactgaacagggctaacatgctttacgtcggcggcggacacgcctacttcgtcctggccaacaccgaaaagactgtggaaaccctggtgcagtttgagaaggatttcaaccagttcctgttggcaaacttccagacccgcctctatgtggcctttggctggggttccttcgcggccaaggacatcatgtccgagctgaatagccccgagtcctaccgccaagtgtaccaaaaggcttcgcgcatgatctccaaaaagaaaatctccagatacgactaccagacactgatgctcctgaatcgcggtggaaagtcctcagagagagagtgcgagatttgccactccgtggagaacctggtgtcctaccacgaccagaaagtctgtgacatttgccggggactgtaccagttctcgaaagaaattgcccatgaccacttcatcattaccgaaaatgaggggctgccgattggaccaaacgcgtgcttaaagggcgtggcattcgaaaagctgtcccaagaagcgttcagccgggtctacgtgaagaatgactataaggccggtaccgtgaaggctacgcatgtgttcgtgggggattaccagtgcgacgagatctacaactacgccgccctgagcaagaacgagaacggcctaggcatcaagagactggccgtggtccggctcgacgtggatgacttgggcgccgccttcatggccggtttcagccagcagggaaacggacaatactccactctgtcaagatcggccacattctcccggagcatgtcgctgttcttcaaagtgtacattaaccagttcgcctccgacaagaagctgagcattatctacgcgggcggcgatgacgtgttcgccattggatcgtggcaggatatcatcgcgttcactgtggaacttcgcgaaaacttcatcaagtggaccaacgggaagctcaccctctccgcggggatagggttgttcgccgacaagactcctattagcctgatggctcaccagaccggggaactggaagaggccgccaagggcaacgaaaaggactccatctcgctgttctcaagcgactacactttcaagtttgataggttcatcactaacgtgtacgacgacaaactggaacagattagatacttcttcaaccatcaagacgagaggggaaagaacttcatctataagcttattgagcttttgaggaaccacgaccgcatgaatatggcacgcctcgcctattacctcactcgcctggaagaactgacccgggagactgacagggacaagttcaagaccttcaagaacctgttctactcctggtacaccaacaagaacgataaggaccggaaggaagccgagctcgcgctcctgctgtacatctacgaaatcagaaaggattaacggcaataaaaagacagaataaaacgcacggtgttgggtcgtttgttcgcacacattagctagccgtcagcacacattgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggattacaaagacgatgacgataagatggcgcctaagaagaaacgcaaagtgcggggcatgaccatcctgaccgacgagaactacgtggacatcgccgagaaagccatcctgaagctggaaagaaacaccagaaatagaaagaaccctgatgccttcttcctgaccacatctaagctgcggaacctgctgagcctgacaagcaccctgttcgacgagagcaaggtgaaggaatacgacgccctgctggacagaatcgcttatctgagagtgcagttcgtgtaccaggccggcagagagatcgccgtgaaagatctgatcgagaaggcccagatcctggaagctctgaaagagatcaaggaccgggaaaccctgcagagattctgcagatacatggaagccctggtggcctacttcaagttctacggcggcaaggactgacggcaataaaaagacagaataaaacgcacggtgttgggtcgtttgttcagttcttttgccttactttcaatgcatgcggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggattacaaagacgatgacgataagatggcgcctaagaagaaacgcaaagtgcggggcatgaccttcgccaagatcaaattcagcgcccagatccggctggaaaccggcctgcacatcggaggatctgatgcctttgccgctatcggcgccatcgacagccctgtgatcaaggaccccatcaccaacctgcctatcatccccggctctagcctgaagggcaagatgagaacactgctggccaaggtgtacaacgaaaaggtggccgagaagcctagcgacgacagcgacatcctgagcagactgttcggaaatagcaaggataagcggttcaagatgggcagactgatcttccgggacgccttcctgagcaacgccgacgagctggattctctgggcgtgcggagctacaccgaggtgaagttcgagaacaccatcgatagaatcaccgccgaggccaatcctagacagatcgagagagccattcggaactcaacattcgacttcgagctgatctacgagatcactgatgagaatgagaaccaggtcgaggaagatttcaaggtgatcagagacggcctgaagctgctggaactggactacctgggcggaagcggctccagaggctacggcaaagtggcttttgagaacctgaaagccaccacagtgttcggcaactacgacgtgaaaaccctgaacgagctgctgaccgccgaagtgtgacggcaataaaaagacagaataaaacgcacggtgttgggtcgtttgttctggattgcgagaatggactagtagcaaactgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggattacaaagacgatgacgataagatggcgcctaagaagaaacgcaaagtgcggggcatgacttacaagctctacattatgacctttcaaaacgcccacttcggttccggcactctggactcatcgaagctgaccttctccgcggatagaatcttctcggcactcgtgctcgaggctctgaagatgggaaagctcgacgccttcttggccgaggccaaccaggataagttcactctgaccgacgcgttcccattccaattcggtcctttcctgccgaaaccgattggttaccccaagcacgaccagatcgaccagtctgtggacgtgaaggaagtccgccgccaagcgaagctgtccaaaaagctccagttcctggctctggaaaacgtcgacgactacctgaacggagagctgtttgagaatgaggaacacgccgtgatcgacacagtgaccaagaaccagccccataaagatgataatctgtaccaagtggccaccactcggttctcgaacgacacctccctttacgtgatcgccaacgaatccgatctgctgaacgaactgatgagcagccttcagtactccgggctgggcggcaaaaggtcctcaggattcggcagatttgagctggacatccagaacattcccttggaactgtccgaccggctgacgaagaaccacagcgacaaggtcatgtcacttaccaccgccctcccggtggacgctgatctcgaggaagcgatggaagatggccattacctgttgaccaagtcgtccggattcgcattctcccacgccaccaacgaaaactatcggaagcaggacctgtacaagttcgcctccgggagcaccttcagcaagactttcgagggacagatcgtggacgtgcgccctctcgatttccctcacgccgtgctgaactacgccaagccgctgttctttaagctcgaagtctaacggcaataaaaagacagaataaaacgcacggtgttgggtcgtttgttcgcacattcaaaaacaggcaattggacaagcgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggattacaaagacgatgacgataagatggcgcctaagaagaaacgcaaagtgcggggcatgaaaaatgactaccggaccttcaagctgagcctgctgaccctggctcctatccacatcggcaacggcgagaagtacaccagcagagaattcatctacgagaacaagaagttctacttccccgacatgggcaagttctacaacaagatggtggaaaagagactggccgagaagttcgaggccttcctgatccagaccagacccaacgccagaaacaaccggctgatttcttttctgaacgacaacagaatcgccgaaagatcttttggcggctacagcatcagtgaaaccggcctggaatctgataagaaccctaacagcgccggagctatcaacgaggtgaacaaattcatccgggacgccttcggaaatccttacatcccaggcagcagcctgaagggcgccatccgcaccatcctgatgaacaccacacctaagtggaacaacgagaacgccgtgaacgacttcggcagattcccaaaggaaaacaagaacctgatcccttggggacctaagaaaggcaaggaatacgacgacctgttcaacgccatcagagtgtccgacagcaagcccttcgacaacaaaagcctgatcctcgtgcagaagtgggactacagcgccaaaaccaacaaggccaagcctctgcctctgtacagagagtctatcagccctctgaccaagatcgagttcgagataacaacaaccactgatgaggccggcagactgatcgaggaactgggaaagcgggcccaggccttttataaggactacaaggcctttttcctgtctgaattccctgatgataagatccaggctaatctgcaataccccatctacctgggcgccggcagcggcgcttggacaaagaccctgtttaagcaggccgacggcatcctgcagcggagatactccagaatgaaaaccaagatggtcaagaagggcgtgctgaagctgacaaaggcccctctgaaaacagtgaagatccccagcggcaaccacagcctggtgaagaatcacgagagcttctacgagatgggcaaagccaacttcatgatcaaggaaatcgacaagtgacggcaataaaaagacagaataaaacgcacggtgttgggtcgtttgttcatgtgtgccggccggagaataaaagtctaagtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggattacaaagacgatgacgataagatggcgcctaagaagaaacgcaaagtgcggggcatgaaaaagctcgtgttcacctttaagcggatcgaccaccctgctcaggacctggccgtgaaattccacggcttcctgatggaacagctggatagcgactacgtggactacctgcaccagcagcagaccaacccctacgccacaaaggtgatccagggcaaagagaacacccagtgggtcgtgcatctgctgacagacgacatcgaggacaaggtgttcatgaccctgctgcagatcaaggaagtgtccctgaacgacctgcctaagttgtctgtggaaaaggtggaaatccaggagctgggcgctgataagctgctcgagatcttcaacagcgaggaaaaccagacctacttcagcatcatcttcgagacacctacaggctttaaaagccagggcagctacgtgatcttccccagcatgcggctgatctttcagagcctgatgcagaagtacggcagactggtggaaaaccagcctgagatcgaggaagataccctggactacctgagcgagcacagcaccatcaccaattacagactggaaacaagctacttcagagtgcatagacagagaatccccgccttccggggcaagctgaccttcaaggtgcagggagcccagacactgaaggcctacgtgaagatgctgctgaccttcggcgagtacagcggcctgggcatgaaaaccagcctgggaatgggcggcatcaagctggaagaaagaaaggactgacggcaataaaaagacagaataaaacgcacggtgttgggtcgtttgttcgtgcgatagagggatcccgcattgaattatgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgacggcaataaaaagacagaataaaacgcacggtgttgggtcgtttgttcgtagatggcgcgcctttgttgacccggttggagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgatataaacctaattacctcgagaggggacggaaacccgtcttcgatgaagcgattcagaagacttgatataaacctaattacctcgagaggggacttttttacatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttcttaagc

SEQ ID NO: 174 - DNA, 11480 bp, synthetic construct
  source 1..11480: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatgcaccatcaccatcaccattccggcgattacaaagacgatgacgataagatggcgcctaagaagaaacgcaaagtgcggggcatgaagaaagaaaagattgatctgttttacggagccctgctgcacgacatcggaaaggtcatccagcgagcaaccggagagcggaagaaacacgcacttgtgggcgccgactggttcgacgagatcgccgacaaccaagtcatctcggatcagatccggtaccatatggccaactaccagtctgataagctcggcaacgatcacctggcttacatcacctacattgccgacaacatcgcctccggtgtcgaccgccggcaatccaacgaagagtcagacgaagatacctccgcaaagatctgggacacctacacgaaccaggccgacatctttaacgtgttcggagcgcagaccgataagcggtacttcaagcctaccgtgctgaatctcaagtcgaagcccaacttcgcgtccgccacttacgaaccctttagcaagggcgattacgctgccatcgccacccggattaagaacgaactggccgagttcgagttcaaccaagtccagattgactccctgctcaaccttttcgaggctactctctccttcgtgccgtcaagcaccaacactaaggaaatcgccgacatctccctggccgaccattcccgcttgactgctgccttcgctctggcgatctacgactacctggaggacaagggtcggcacaactacaaagaggacctgttcaccaaagtgtcagcgttctatgaagaagaagccttcctgctggcctccttcgacctgtcgggaatccaggactttatctacaacattaacatcgcaactaacggcgcggcgaagcagctgaaggcccggagcctctacctggactttatgtccgagtacatcgccgatagcctgctggacaagctgggactgaacagggctaacatgctttacgtcggcggcggacacgcctacttcgtcctggccaacaccgaaaagactgtggaaaccctggtgcagtttgagaaggatttcaaccagttcctgttggcaaacttccagacccgcctctatgtggcctttggctggggttccttcgcggccaaggacatcatgtccgagctgaatagccccgagtcctaccgccaagtgtaccaaaaggcttcgcgcatgatctccaaaaagaaaatctccagatacgactaccagacactgatgctcctgaatcgcggtggaaagtcctcagagagagagtgcgagatttgccactccgtggagaacctggtgtcctaccacgaccagaaagtctgtgacatttgccggggactgtaccagttctcgaaagaaattgcccatgaccacttcatcattaccgaaaatgaggggctgccgattggaccaaacgcgtgcttaaagggcgtggcattcgaaaagctgtcccaagaagcgttcagccgggtctacgtgaagaatgactataaggccggtaccgtgaaggctacgcatgtgttcgtgggggattaccagtgcgacgagatctacaactacgccgccctgagcaagaacgagaacggcctaggcatcaagagactggccgtggtccggctcgacgtggatgacttgggcgccgccttcatggccggtttcagccagcagggaaacggacaatactccactctgtcaagatcggccacattctcccggagcatgtcgctgttcttcaaagtgtacattaaccagttcgcctccgacaagaagctgagcattatctacgcgggcggcgatgacgtgttcgccattggatcgtggcaggatatcatcgcgttcactgtggaacttcgcgaaaacttcatcaagtggaccaacgggaagctcaccctctccgcggggatagggttgttcgccgacaagactcctattagcctgatggctcaccagaccggggaactggaagaggccgccaagggcaacgaaaaggactccatctcgctgttctcaagcgactacactttcaagtttgataggttcatcactaacgtgtacgacgacaaactggaacagattagatacttcttcaaccatcaagacgagaggggaaagaacttcatctataagcttattgagcttttgaggaaccacgaccgcatgaatatggcacgcctcgcctattacctcactcgcctggaagaactgacccgggagactgacagggacaagttcaagaccttcaagaacctgttctactcctggtacaccaacaagaacgataaggaccggaaggaagccgagctcgcgctcctgctgtacatctacgaaatcagaaaggattaacggcaataaaaagacagaataaaacgcacggtgttgggtcgtttgttcgcacacattagctagccgtcagcacacattgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggattacaaagacgatgacgataagatggcgcctaagaagaaacgcaaagtgcggggcatgaccatcctgaccgacgagaactacgtggacatcgccgagaaagccatcctgaagctggaaagaaacaccagaaatagaaagaaccctgatgccttcttcctgaccacatctaagctgcggaacctgctgagcctgacaagcaccctgttcgacgagagcaaggtgaaggaatacgacgccctgctggacagaatcgcttatctgagagtgcagttcgtgtaccaggccggcagagagatcgccgtgaaagatctgatcgagaaggcccagatcctggaagctctgaaagagatcaaggaccgggaaaccctgcagagattctgcagatacatggaagccctggtggcctacttcaagttctacggcggcaaggactgacggcaataaaaagacagaataaaacgcacggtgttgggtcgtttgttcagttcttttgccttactttcaatgcatgcggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggattacaaagacgatgacgataagatggcgcctaagaagaaacgcaaagtgcggggcatgaccttcgccaagatcaaattcagcgcccagatccggctggaaaccggcctgcacatcggaggatctgatgcctttgccgctatcggcgccatcgacagccctgtgatcaaggaccccatcaccaacctgcctatcatccccggctctagcctgaagggcaagatgagaacactgctggccaaggtgtacaacgaaaaggtggccgagaagcctagcgacgacagcgacatcctgagcagactgttcggaaatagcaaggataagcggttcaagatgggcagactgatcttccgggacgccttcctgagcaacgccgacgagctggattctctgggcgtgcggagctacaccgaggtgaagttcgagaacaccatcgatagaatcaccgccgaggccaatcctagacagatcgagagagccattcggaactcaacattcgacttcgagctgatctacgagatcactgatgagaatgagaaccaggtcgaggaagatttcaaggtgatcagagacggcctgaagctgctggaactggactacctgggcggaagcggctccagaggctacggcaaagtggcttttgagaacctgaaagccaccacagtgttcggcaactacgacgtgaaaaccctgaacgagctgctgaccgccgaagtgtgacggcaataaaaagacagaataaaacgcacggtgttgggtcgtttgttctggattgcgagaatggactagtagcaaactgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggattacaaagacgatgacgataagatggcgcctaagaagaaacgcaaagtgcggggcatgacttacaagctctacattatgacctttcaaaacgcccacttcggttccggcactctggactcatcgaagctgaccttctccgcggatagaatcttctcggcactcgtgctcgaggctctgaagatgggaaagctcgacgccttcttggccgaggccaaccaggataagttcactctgaccgacgcgttcccattccaattcggtcctttcctgccgaaaccgattggttaccccaagcacgaccagatcgaccagtctgtggacgtgaaggaagtccgccgccaagcgaagctgtccaaaaagctccagttcctggctctggaaaacgtcgacgactacctgaacggagagctgtttgagaatgaggaacacgccgtgatcgacacagtgaccaagaaccagccccataaagatgataatctgtaccaagtggccaccactcggttctcgaacgacacctccctttacgtgatcgccaacgaatccgatctgctgaacgaactgatgagcagccttcagtactccgggctgggcggcaaaaggtcctcaggattcggcagatttgagctggacatccagaacattcccttggaactgtccgaccggctgacgaagaaccacagcgacaaggtcatgtcacttaccaccgccctcccggtggacgctgatctcgaggaagcgatggaagatggccattacctgttgaccaagtcgtccggattcgcattctcccacgccaccaacgaaaactatcggaagcaggacctgtacaagttcgcctccgggagcaccttcagcaagactttcgagggacagatcgtggacgtgcgccctctcgatttccctcacgccgtgctgaactacgccaagccgctgttctttaagctcgaagtctaacggcaataaaaagacagaataaaacgcacggtgttgggtcgtttgttcgcacattcaaaaacaggcaattggacaagcgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggattacaaagacgatgacgataagatggcgcctaagaagaaacgcaaagtgcggggcatgaaaaatgactaccggaccttcaagctgagcctgctgaccctggctcctatccacatcggcaacggcgagaagtacaccagcagagaattcatctacgagaacaagaagttctacttccccgacatgggcaagttctacaacaagatggtggaaaagagactggccgagaagttcgaggccttcctgatccagaccagacccaacgccagaaacaaccggctgatttcttttctgaacgacaacagaatcgccgaaagatcttttggcggctacagcatcagtgaaaccggcctggaatctgataagaaccctaacagcgccggagctatcaacgaggtgaacaaattcatccgggacgccttcggaaatccttacatcccaggcagcagcctgaagggcgccatccgcaccatcctgatgaacaccacacctaagtggaacaacgagaacgccgtgaacgacttcggcagattcccaaaggaaaacaagaacctgatcccttggggacctaagaaaggcaaggaatacgacgacctgttcaacgccatcagagtgtccgacagcaagcccttcgacaacaaaagcctgatcctcgtgcagaagtgggactacagcgccaaaaccaacaaggccaagcctctgcctctgtacagagagtctatcagccctctgaccaagatcgagttcgagataacaacaaccactgatgaggccggcagactgatcgaggaactgggaaagcgggcccaggccttttataaggactacaaggcctttttcctgtctgaattccctgatgataagatccaggctaatctgcaataccccatctacctgggcgccggcagcggcgcttggacaaagaccctgtttaagcaggccgacggcatcctgcagcggagatactccagaatgaaaaccaagatggtcaagaagggcgtgctgaagctgacaaaggcccctctgaaaacagtgaagatccccagcggcaaccacagcctggtgaagaatcacgagagcttctacgagatgggcaaagccaacttcatgatcaaggaaatcgacaagtgacggcaataaaaagacagaataaaacgcacggtgttgggtcgtttgttcatgtgtgccggccggagaataaaagtctaagtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggattacaaagacgatgacgataagatggcgcctaagaagaaacgcaaagtgcggggcatgaaaaagctcgtgttcacctttaagcggatcgaccaccctgctcaggacctggccgtgaaattccacggcttcctgatggaacagctggatagcgactacgtggactacctgcaccagcagcagaccaacccctacgccacaaaggtgatccagggcaaagagaacacccagtgggtcgtgcatctgctgacagacgacatcgaggacaaggtgttcatgaccctgctgcagatcaaggaagtgtccctgaacgacctgcctaagttgtctgtggaaaaggtggaaatccaggagctgggcgctgataagctgctcgagatcttcaacagcgaggaaaaccagacctacttcagcatcatcttcgagacacctacaggctttaaaagccagggcagctacgtgatcttccccagcatgcggctgatctttcagagcctgatgcagaagtacggcagactggtggaaaaccagcctgagatcgaggaagataccctggactacctgagcgagcacagcaccatcaccaattacagactggaaacaagctacttcagagtgcatagacagagaatccccgccttccggggcaagctgaccttcaaggtgcagggagcccagacactgaaggcctacgtgaagatgctgctgaccttcggcgagtacagcggcctgggcatgaaaaccagcctgggaatgggcggcatcaagctggaagaaagaaaggactgacggcaataaaaagacagaataaaacgcacggtgttgggtcgtttgttcgtgcgatagagggatcccgcattgaattatgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggcggagcgcgccggggtgcccgccttcctggaaacctccgcgccccgcaacctccccttctacgagcggctcggcttcaccgtcaccgccgacgtcgaggtgcccgaaggaccgcgcacctggtgcatgacccgcaagcccggtgcctgacggcaataaaaagacagaataaaacgcacggtgttgggtcgtttgttcgagcagattgtactgagagtgcaccggttggagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgatataaacctaattacctcgagaggggacggaaacccgtcttcgatgaagcgattcagaagacttgatataaacctaattacctcgagaggggacttttttacatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttcttaagc

SEQ ID NO: 175 - DNA, 11600 bp, synthetic construct
  source 1..11600: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatgcaccatcaccatcaccattccggcgattacaaagacgatgacgataagatggcgcctaagaagaaacgcaaagtgcggggcatgaagaaagaaaagattgatctgttttacggagccctgctgcacgacatcggaaaggtcatccagcgagcaaccggagagcggaagaaacacgcacttgtgggcgccgactggttcgacgagatcgccgacaaccaagtcatctcggatcagatccggtaccatatggccaactaccagtctgataagctcggcaacgatcacctggcttacatcacctacattgccgacaacatcgcctccggtgtcgaccgccggcaatccaacgaagagtcagacgaagatacctccgcaaagatctgggacacctacacgaaccaggccgacatctttaacgtgttcggagcgcagaccgataagcggtacttcaagcctaccgtgctgaatctcaagtcgaagcccaacttcgcgtccgccacttacgaaccctttagcaagggcgattacgctgccatcgccacccggattaagaacgaactggccgagttcgagttcaaccaagtccagattgactccctgctcaaccttttcgaggctactctctccttcgtgccgtcaagcaccaacactaaggaaatcgccgacatctccctggccgaccattcccgcttgactgctgccttcgctctggcgatctacgactacctggaggacaagggtcggcacaactacaaagaggacctgttcaccaaagtgtcagcgttctatgaagaagaagccttcctgctggcctccttcgacctgtcgggaatccaggactttatctacaacattaacatcgcaactaacggcgcggcgaagcagctgaaggcccggagcctctacctggactttatgtccgagtacatcgccgatagcctgctggacaagctgggactgaacagggctaacatgctttacgtcggcggcggacacgcctacttcgtcctggccaacaccgaaaagactgtggaaaccctggtgcagtttgagaaggatttcaaccagttcctgttggcaaacttccagacccgcctctatgtggcctttggctggggttccttcgcggccaaggacatcatgtccgagctgaatagccccgagtcctaccgccaagtgtaccaaaaggcttcgcgcatgatctccaaaaagaaaatctccagatacgactaccagacactgatgctcctgaatcgcggtggaaagtcctcagagagagagtgcgagatttgccactccgtggagaacctggtgtcctaccacgaccagaaagtctgtgacatttgccggggactgtaccagttctcgaaagaaattgcccatgaccacttcatcattaccgaaaatgaggggctgccgattggaccaaacgcgtgcttaaagggcgtggcattcgaaaagctgtcccaagaagcgttcagccgggtctacgtgaagaatgactataaggccggtaccgtgaaggctacgcatgtgttcgtgggggattaccagtgcgacgagatctacaactacgccgccctgagcaagaacgagaacggcctaggcatcaagagactggccgtggtccggctcgacgtggatgacttgggcgccgccttcatggccggtttcagccagcagggaaacggacaatactccactctgtcaagatcggccacattctcccggagcatgtcgctgttcttcaaagtgtacattaaccagttcgcctccgacaagaagctgagcattatctacgcgggcggcgatgacgtgttcgccattggatcgtggcaggatatcatcgcgttcactgtggaacttcgcgaaaacttcatcaagtggaccaacgggaagctcaccctctccgcggggatagggttgttcgccgacaagactcctattagcctgatggctcaccagaccggggaactggaagaggccgccaagggcaacgaaaaggactccatctcgctgttctcaagcgactacactttcaagtttgataggttcatcactaacgtgtacgacgacaaactggaacagattagatacttcttcaaccatcaagacgagaggggaaagaacttcatctataagcttattgagcttttgaggaaccacgaccgcatgaatatggcacgcctcgcctattacctcactcgcctggaagaactgacccgggagactgacagggacaagttcaagaccttcaagaacctgttctactcctggtacaccaacaagaacgataaggaccggaaggaagccgagctcgcgctcctgctgtacatctacgaaatcagaaaggattaacggcaataaaaagacagaataaaacgcacggtgttgggtcgtttgttcgcacacattagctagccgtcagcacacattgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggattacaaagacgatgacgataagatggcgcctaagaagaaacgcaaagtgcggggcatgaccatcctgaccgacgagaactacgtggacatcgccgagaaagccatcctgaagctggaaagaaacaccagaaatagaaagaaccctgatgccttcttcctgaccacatctaagctgcggaacctgctgagcctgacaagcaccctgttcgacgagagcaaggtgaaggaatacgacgccctgctggacagaatcgcttatctgagagtgcagttcgtgtaccaggccggcagagagatcgccgtgaaagatctgatcgagaaggcccagatcctggaagctctgaaagagatcaaggaccgggaaaccctgcagagattctgcagatacatggaagccctggtggcctacttcaagttctacggcggcaaggactgacggcaataaaaagacagaataaaacgcacggtgttgggtcgtttgttcagttcttttgccttactttcaatgcatgcggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggattacaaagacgatgacgataagatggcgcctaagaagaaacgcaaagtgcggggcatgaccttcgccaagatcaaattcagcgcccagatccggctggaaaccggcctgcacatcggaggatctgatgcctttgccgctatcggcgccatcgccagccctgtgatcaaggaccccatcaccaacctgcctatcatccccggctctagcctgaagggcaagatgagaacactgctggccaaggtgtacaacgaaaaggtggccgagaagcctagcgacgacagcgacatcctgagcagactgttcggaaatagcaaggataagcggttcaagatgggcagactgatcttccgggacgccttcctgagcaacgccgacgagctggattctctgggcgtgcggagctacaccgaggtgaagttcgagaacaccatcgatagaatcaccgccgaggccaatcctagacagatcgagagagccattcggaactcaacattcgacttcgagctgatctacgagatcactgatgagaatgagaaccaggtcgaggaagatttcaaggtgatcagagacggcctgaagctgctggaactggactacctgggcggaagcggctccagaggctacggcaaagtggcttttgagaacctgaaagccaccacagtgttcggcaactacgacgtgaaaaccctgaacgagctgctgaccgccgaagtgtgacggcaataaaaagacagaataaaacgcacggtgttgggtcgtttgttctggattgcgagaatggactagtagcaaactgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggattacaaagacgatgacgataagatggcgcctaagaagaaacgcaaagtgcggggcatgacttacaagctctacattatgacctttcaaaacgcccacttcggttccggcactctggactcatcgaagctgaccttctccgcggatagaatcttctcggcactcgtgctcgaggctctgaagatgggaaagctcgacgccttcttggccgaggccaaccaggataagttcactctgaccgacgcgttcccattccaattcggtcctttcctgccgaaaccgattggttaccccaagcacgaccagatcgaccagtctgtggacgtgaaggaagtccgccgccaagcgaagctgtccaaaaagctccagttcctggctctggaaaacgtcgacgactacctgaacggagagctgtttgagaatgaggaacacgccgtgatcgacacagtgaccaagaaccagccccataaagatgataatctgtaccaagtggccaccactcggttctcgaacgacacctccctttacgtgatcgccaacgaatccgatctgctgaacgaactgatgagcagccttcagtactccgggctgggcggcaaaaggtcctcaggattcggcagatttgagctggacatccagaacattcccttggaactgtccgaccggctgacgaagaaccacagcgacaaggtcatgtcacttaccaccgccctcccggtggacgctgatctcgaggaagcgatggaagatggccattacctgttgaccaagtcgtccggattcgcattctcccacgccaccaacgaaaactatcggaagcaggacctgtacaagttcgcctccgggagcaccttcagcaagactttcgagggacagatcgtggacgtgcgccctctcgatttccctcacgccgtgctgaactacgccaagccgctgttctttaagctcgaagtctaacggcaataaaaagacagaataaaacgcacggtgttgggtcgtttgttcgcacattcaaaaacaggcaattggacaagcgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggattacaaagacgatgacgataagatggcgcctaagaagaaacgcaaagtgcggggcatgaaaaatgactaccggaccttcaagctgagcctgctgaccctggctcctatccacatcggcaacggcgagaagtacaccagcagagaattcatctacgagaacaagaagttctacttccccgacatgggcaagttctacaacaagatggtggaaaagagactggccgagaagttcgaggccttcctgatccagaccagacccaacgccagaaacaaccggctgatttcttttctgaacgacaacagaatcgccgaaagatcttttggcggctacagcatcagtgaaaccggcctggaatctgataagaaccctaacagcgccggagctatcaacgaggtgaacaaattcatccgggacgccttcggaaatccttacatcccaggcagcagcctgaagggcgccatccgcaccatcctgatgaacaccacacctaagtggaacaacgagaacgccgtgaacgacttcggcagattcccaaaggaaaacaagaacctgatcccttggggacctaagaaaggcaaggaatacgacgacctgttcaacgccatcagagtgtccgacagcaagcccttcgacaacaaaagcctgatcctcgtgcagaagtgggactacagcgccaaaaccaacaaggccaagcctctgcctctgtacagagagtctatcagccctctgaccaagatcgagttcgagataacaacaaccactgatgaggccggcagactgatcgaggaactgggaaagcgggcccaggccttttataaggactacaaggcctttttcctgtctgaattccctgatgataagatccaggctaatctgcaataccccatctacctgggcgccggcagcggcgcttggacaaagaccctgtttaagcaggccgacggcatcctgcagcggagatactccagaatgaaaaccaagatggtcaagaagggcgtgctgaagctgacaaaggcccctctgaaaacagtgaagatccccagcggcaaccacagcctggtgaagaatcacgagagcttctacgagatgggcaaagccaacttcatgatcaaggaaatcgacaagtgacggcaataaaaagacagaataaaacgcacggtgttgggtcgtttgttcatgtgtgccggccggagaataaaagtctaagtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggattacaaagacgatgacgataagatggcgcctaagaagaaacgcaaagtgcggggcatgaaaaagctcgtgttcacctttaagcggatcgaccaccctgctcaggacctggccgtgaaattccacggcttcctgatggaacagctggatagcgactacgtggactacctgcaccagcagcagaccaacccctacgccacaaaggtgatccagggcaaagagaacacccagtgggtcgtgcatctgctgacagacgacatcgaggacaaggtgttcatgaccctgctgcagatcaaggaagtgtccctgaacgacctgcctaagttgtctgtggaaaaggtggaaatccaggagctgggcgctgataagctgctcgagatcttcaacagcgaggaaaaccagacctacttcagcatcatcttcgagacacctacaggctttaaaagccagggcagctacgtgatcttccccagcatgcggctgatctttcagagcctgatgcagaagtacggcagactggtggaaaaccagcctgagatcgaggaagataccctggactacctgagcgagcacagcaccatcaccaattacagactggaaacaagctacttcagagtgcatagacagagaatccccgccttccggggcaagctgaccttcaaggtgcagggagcccagacactgaaggcctacgtgaagatgctgctgaccttcggcgagtacagcggcctgggcatgaaaaccagcctgggaatgggcggcatcaagctggaagaaagaaaggactgacggcaataaaaagacagaataaaacgcacggtgttgggtcgtttgttcgtgcgatagagggatcccgcattgaattatgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgacggcaataaaaagacagaataaaacgcacggtgttgggtcgtttgttcgtagatggcgcgcctttgttgacccggttggagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgatataaacctaattacctcgagaggggacggaaacccgtcttcgatgaagcgattcagaagacttgatataaacctaattacctcgagaggggacttttttacatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttcttaagc

SEQ ID NO: 176 - DNA, 11297 bp, synthetic construct
  source 1..11297: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatgcaccatcaccatcaccattccggcgattacaaagacgatgacgataagatggcgcctaagaagaaacgcaaagtgcggggcatgaagaaagaaaagattgatctgttttacggagccctgctgcacgacatcggaaaggtcatccagcgagcaaccggagagcggaagaaacacgcacttgtgggcgccgactggttcgacgagatcgccgacaaccaagtcatctcggatcagatccggtaccatatggccaactaccagtctgataagctcggcaacgatcacctggcttacatcacctacattgccgacaacatcgcctccggtgtcgaccgccggcaatccaacgaagagtcagacgaagatacctccgcaaagatctgggacacctacacgaaccaggccgacatctttaacgtgttcggagcgcagaccgataagcggtacttcaagcctaccgtgctgaatctcaagtcgaagcccaacttcgcgtccgccacttacgaaccctttagcaagggcgattacgctgccatcgccacccggattaagaacgaactggccgagttcgagttcaaccaagtccagattgactccctgctcaaccttttcgaggctactctctccttcgtgccgtcaagcaccaacactaaggaaatcgccgacatctccctggccgaccattcccgcttgactgctgccttcgctctggcgatctacgactacctggaggacaagggtcggcacaactacaaagaggacctgttcaccaaagtgtcagcgttctatgaagaagaagccttcctgctggcctccttcgacctgtcgggaatccaggactttatctacaacattaacatcgcaactaacggcgcggcgaagcagctgaaggcccggagcctctacctggactttatgtccgagtacatcgccgatagcctgctggacaagctgggactgaacagggctaacatgctttacgtcggcggcggacacgcctacttcgtcctggccaacaccgaaaagactgtggaaaccctggtgcagtttgagaaggatttcaaccagttcctgttggcaaacttccagacccgcctctatgtggcctttggctggggttccttcgcggccaaggacatcatgtccgagctgaatagccccgagtcctaccgccaagtgtaccaaaaggcttcgcgcatgatctccaaaaagaaaatctccagatacgactaccagacactgatgctcctgaatcgcggtggaaagtcctcagagagagagtgcgagatttgccactccgtggagaacctggtgtcctaccacgaccagaaagtctgtgacatttgccggggactgtaccagttctcgaaagaaattgcccatgaccacttcatcattaccgaaaatgaggggctgccgattggaccaaacgcgtgcttaaagggcgtggcattcgaaaagctgtcccaagaagcgttcagccgggtctacgtgaagaatgactataaggccggtaccgtgaaggctacgcatgtgttcgtgggggattaccagtgcgacgagatctacaactacgccgccctgagcaagaacgagaacggcctaggcatcaagagactggccgtggtccggctcgacgtggatgacttgggcgccgccttcatggccggtttcagccagcagggaaacggacaatactccactctgtcaagatcggccacattctcccggagcatgtcgctgttcttcaaagtgtacattaaccagttcgcctccgacaagaagctgagcattatctacgcgggcggcgatgacgtgttcgccattggatcgtggcaggatatcatcgcgttcactgtggaacttcgcgaaaacttcatcaagtggaccaacgggaagctcaccctctccgcggggatagggttgttcgccgacaagactcctattagcctgatggctcaccagaccggggaactggaagaggccgccaagggcaacgaaaaggactccatctcgctgttctcaagcgactacactttcaagtttgataggttcatcactaacgtgtacgacgacaaactggaacagattagatacttcttcaaccatcaagacgagaggggaaagaacttcatctataagcttattgagcttttgaggaaccacgaccgcatgaatatggcacgcctcgcctattacctcactcgcctggaagaactgacccgggagactgacagggacaagttcaagaccttcaagaacctgttctactcctggtacaccaacaagaacgataaggaccggaaggaagccgagctcgcgctcctgctgtacatctacgaaatcagaaaggattaacggcaataaaaagacagaataaaacgcacggtgttgggtcgtttgttcgcacacattagctagccgtcagcacacattgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggattacaaagacgatgacgataagatggcgcctaagaagaaacgcaaagtgcggggcatgaccatcctgaccgacgagaactacgtggacatcgccgagaaagccatcctgaagctggaaagaaacaccagaaatagaaagaaccctgatgccttcttcctgaccacatctaagctgcggaacctgctgagcctgacaagcaccctgttcgacgagagcaaggtgaaggaatacgacgccctgctggacagaatcgcttatctgagagtgcagttcgtgtaccaggccggcagagagatcgccgtgaaagatctgatcgagaaggcccagatcctggaagctctgaaagagatcaaggaccgggaaaccctgcagagattctgcagatacatggaagccctggtggcctacttcaagttctacggcggcaaggactgacggcaataaaaagacagaataaaacgcacggtgttgggtcgtttgttcagttcttttgccttactttcaatgcatgcggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggattacaaagacgatgacgataagatggcgcctaagaagaaacgcaaagtgcggggcatgaccttcgccaagatcaaattcagcgcccagatccggctggaaaccggcctgcacatcggaggatctgatgcctttgccgctatcggcgccatcgccagccctgtgatcaaggaccccatcaccaacctgcctatcatccccggctctagcctgaagggcaagatgagaacactgctggccaaggtgtacaacgaaaaggtggccgagaagcctagcgacgacagcgacatcctgagcagactgttcggaaatagcaaggataagcggttcaagatgggcagactgatcttccgggacgccttcctgagcaacgccgacgagctggattctctgggcgtgcggagctacaccgaggtgaagttcgagaacaccatcgatagaatcaccgccgaggccaatcctagacagatcgagagagccattcggaactcaacattcgacttcgagctgatctacgagatcactgatgagaatgagaaccaggtcgaggaagatttcaaggtgatcagagacggcctgaagctgctggaactggactacctgggcggaagcggctccagaggctacggcaaagtggcttttgagaacctgaaagccaccacagtgttcggcaactacgacgtgaaaaccctgaacgagctgctgaccgccgaagtgggcggaggtgctgccgcaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgacggcaataaaaagacagaataaaacgcacggtgttgggtcgtttgttctggattgcgagaatggactagtagcaaactgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggattacaaagacgatgacgataagatggcgcctaagaagaaacgcaaagtgcggggcatgacttacaagctctacattatgacctttcaaaacgcccacttcggttccggcactctggactcatcgaagctgaccttctccgcggatagaatcttctcggcactcgtgctcgaggctctgaagatgggaaagctcgacgccttcttggccgaggccaaccaggataagttcactctgaccgacgcgttcccattccaattcggtcctttcctgccgaaaccgattggttaccccaagcacgaccagatcgaccagtctgtggacgtgaaggaagtccgccgccaagcgaagctgtccaaaaagctccagttcctggctctggaaaacgtcgacgactacctgaacggagagctgtttgagaatgaggaacacgccgtgatcgacacagtgaccaagaaccagccccataaagatgataatctgtaccaagtggccaccactcggttctcgaacgacacctccctttacgtgatcgccaacgaatccgatctgctgaacgaactgatgagcagccttcagtactccgggctgggcggcaaaaggtcctcaggattcggcagatttgagctggacatccagaacattcccttggaactgtccgaccggctgacgaagaaccacagcgacaaggtcatgtcacttaccaccgccctcccggtggacgctgatctcgaggaagcgatggaagatggccattacctgttgaccaagtcgtccggattcgcattctcccacgccaccaacgaaaactatcggaagcaggacctgtacaagttcgcctccgggagcaccttcagcaagactttcgagggacagatcgtggacgtgcgccctctcgatttccctcacgccgtgctgaactacgccaagccgctgttctttaagctcgaagtctaacggcaataaaaagacagaataaaacgcacggtgttgggtcgtttgttcgcacattcaaaaacaggcaattggacaagcgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggattacaaagacgatgacgataagatggcgcctaagaagaaacgcaaagtgcggggcatgaaaaatgactaccggaccttcaagctgagcctgctgaccctggctcctatccacatcggcaacggcgagaagtacaccagcagagaattcatctacgagaacaagaagttctacttccccgacatgggcaagttctacaacaagatggtggaaaagagactggccgagaagttcgaggccttcctgatccagaccagacccaacgccagaaacaaccggctgatttcttttctgaacgacaacagaatcgccgaaagatcttttggcggctacagcatcagtgaaaccggcctggaatctgataagaaccctaacagcgccggagctatcaacgaggtgaacaaattcatccgggacgccttcggaaatccttacatcccaggcagcagcctgaagggcgccatccgcaccatcctgatgaacaccacacctaagtggaacaacgagaacgccgtgaacgacttcggcagattcccaaaggaaaacaagaacctgatcccttggggacctaagaaaggcaaggaatacgacgacctgttcaacgccatcagagtgtccgacagcaagcccttcgacaacaaaagcctgatcctcgtgcagaagtgggactacagcgccaaaaccaacaaggccaagcctctgcctctgtacagagagtctatcagccctctgaccaagatcgagttcgagataacaacaaccactgatgaggccggcagactgatcgaggaactgggaaagcgggcccaggccttttataaggactacaaggcctttttcctgtctgaattccctgatgataagatccaggctaatctgcaataccccatctacctgggcgccggcagcggcgcttggacaaagaccctgtttaagcaggccgacggcatcctgcagcggagatactccagaatgaaaaccaagatggtcaagaagggcgtgctgaagctgacaaaggcccctctgaaaacagtgaagatccccagcggcaaccacagcctggtgaagaatcacgagagcttctacgagatgggcaaagccaacttcatgatcaaggaaatcgacaagtgacggcaataaaaagacagaataaaacgcacggtgttgggtcgtttgttcatgtgtgccggccggagaataaaagtctaagtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggattacaaagacgatgacgataagatggcgcctaagaagaaacgcaaagtgcggggcatgaaaaagctcgtgttcacctttaagcggatcgaccaccctgctcaggacctggccgtgaaattccacggcttcctgatggaacagctggatagcgactacgtggactacctgcaccagcagcagaccaacccctacgccacaaaggtgatccagggcaaagagaacacccagtgggtcgtgcatctgctgacagacgacatcgaggacaaggtgttcatgaccctgctgcagatcaaggaagtgtccctgaacgacctgcctaagttgtctgtggaaaaggtggaaatccaggagctgggcgctgataagctgctcgagatcttcaacagcgaggaaaaccagacctacttcagcatcatcttcgagacacctacaggctttaaaagccagggcagctacgtgatcttccccagcatgcggctgatctttcagagcctgatgcagaagtacggcagactggtggaaaaccagcctgagatcgaggaagataccctggactacctgagcgagcacagcaccatcaccaattacagactggaaacaagctacttcagagtgcatagacagagaatccccgccttccggggcaagctgaccttcaaggtgcagggagcccagacactgaaggcctacgtgaagatgctgctgaccttcggcgagtacagcggcctgggcatgaaaaccagcctgggaatgggcggcatcaagctggaagaaagaaaggactgacggcaataaaaagacagaataaaacgcacggtgttgggtcgtttgttcgtgcgatagagggatccagtgcaccggttggagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgatataaacctaattacctcgagaggggacggaaacccgtcttcgatgaagcgattcagaagacttgatataaacctaattacctcgagaggggacttttttacatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttcttaagc

SEQ ID NO: 177 - DNA, 6513 bp, synthetic construct
  source 1..6513: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatgcccaagaagaagagaaaggtggaggccagcatcgagaagaagaagagcttcgccaagggcatgggagtgaagagcaccctggtgtccggctctaaggtgtacatgaccacatttgctgagggaagcgacgccaggctggagaagatcgtggagggcgatagcatcagatccgtgaacgagggagaggctttcagcgccgagatggctgacaagaacgctggctacaagatcggaaacgccaagttttcccacccaaagggctacgccgtggtggctaacaacccactgtacaccggaccagtgcagcaggacatgctgggactgaaggagacactggagaagaggtacttcggcgagtccgccgacggaaacgataacatctgcatccaggtcatccacaacatcctggatatcgagaagatcctggctgagtacatcacaaacgccgcttacgccgtgaacaacatctccggcctggacaaggatatcatcggcttcggaaagttttctaccgtgtacacatacgacgagttcaaggatccagagcaccaccgggccgcttttaacaacaacgacaagctgatcaacgccatcaaggctcagtacgacgagttcgataactttctggataaccccaggctgggctacttcggacaggctttcttttctaaggagggcagaaactacatcatcaactacggaaacgagtgttacgatatcctggcgctgctgagcggactgaggcactgggtggtgcacaacaacgaggaggagtctcggatctctcgcacctggctgtacaacctggacaagaacctggataacgagtacatctccacactgaactacctgtacgacaggatcaccaacgagctgacaaacagcttctccaagaactctgccgctaacgtgaactacatcgctgagaccctgggcatcaacccagctgagttcgctgagcagtacttcagattttccatcatgaaggagcagaagaacctgggcttcaacatcacaaagctgagagaagtgatgctggacagaaaggatatgtccgagatcaggaagaaccacaaggtgttcgattctatcagaaccaaggtgtacacaatgatggactttgtgatctacaggtactacatcgaggaggatgccaaggtggccgctgccaacaagagcctgcccgacaacgagaagtctctgagcgagaaggatatcttcgtgatcaacctgagaggctcctttaacgacgatcagaaggacgctctgtactacgatgaggccaacaggatctggagaaagctggagaacatcatgcacaacatcaaggagttccggggaaacaagacccgcgagtacaagaagaaggacgctccaaggctgcctaggatcctgcctgctggaagggacgtgagcgccttcagcaagctgatgtacgccctgacaatgtttctggacggaaaggagatcaacgatctgctgaccacactgatcaacaagttcgacaacatccagtcttttctgaaagtgatgcctctgatcggcgtgaacgctaagttcgtggaggagtacgccttctttaaggacagcgccaagatcgctgatgagctgcggctgatcaagtcctttgccaggatgggagagccaatcgctgacgctaggagagctatgtacatcgatgccatccggatcctgggaaccaacctgtcttacgacgagctgaaggctctggccgacaccttcagcctggatgagaacggcaacaagctgaagaagggcaagcacggaatgcgcaacttcatcatcaacaacgtgatcagcaacaagcggtttcactacctgatcagatacggcgacccagctcacctgcacgagatcgctaagaacgaggccgtggtgaagttcgtgctgggacggatcgccgatatccagaagaagcagggccagaacggaaagaaccagatcgaccgctactacgagacctgcatcggcaaggataagggaaagtccgtgtctgagaaggtggacgctctgaccaagatcatcacaggcatgaactacgaccagttcgataagaagagatctgtgatcgaggacaccggaagggagaacgccgagagagagaagtttaagaagatcatcagcctgtacctgacagtgatctaccacatcctgaagaacatcgtgaacatcaacgctagatacgtgatcggcttccactgcgtggagcgcgatgcccagctgtacaaggagaagggatacgacatcaacctgaagaagctggaggagaagggctttagctccgtgaccaagctgtgcgctggaatcgacgagacagcccccgacaagaggaaggatgtggagaaggagatggccgagagagctaaggagagcatcgactccctggagtctgctaaccctaagctgtacgccaactacatcaagtactccgatgagaagaaggccgaggagtttaccaggcagatcaacagagagaaggccaagaccgctctgaacgcctacctgaggaacacaaagtggaacgtgatcatccgggaggacctgctgcgcatcgataacaagacctgtacactgttccggaacaaggctgtgcacctggaggtggctcgctacgtgcacgcctacatcaacgacatcgccgaggtgaactcctactttcagctgtaccactacatcatgcagaggatcatcatgaacgagagatacgagaagtctagcggcaaggtgtctgagtacttcgacgccgtgaacgatgagaagaagtacaacgatagactgctgaagctgctgtgcgtgcctttcggatactgtatcccacggtttaagaacctgagcatcgaggccctgttcgaccgcaacgaggctgccaagtttgataaggagaagaagaaggtgagcggcaactccggatccggacctaagaaaaagaggaaggtgtgacggcaataaaaagacagaataaaacgcacggtgttgggtcgtttgttcgtgcgatagagggatcccgcattgaattatgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaaaaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaacggcaataaaaagacagaataaaacgcacggtgttgggtcgtttgttcgagcagattgtactgagagtgcaccggttggagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcaagtaaacccctaccaactggtcggggtttgaaacgggtcttcgagaagacccttttttacatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttcttaagc

SEQ ID NO: 178 - DNA, 3201 bp, synthetic construct
  source 1..3201: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctctagagccgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaaaaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaacggcaataaaaagacagaataaaacgcacggtgttgggtcgtttgttcgagcagattgtactgagagtgcaccggttggagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcgtcttccctgacccagaagacgcttttttacatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttcttaagc

SEQ ID NO: 179 - protein, 343 aa, Archaeoglobus fulgidus
MKSNVVVKYPVNSELANYAEKFWKKELAIYNLSLILNKMTPFVKRRSESYKSSLSAVKEIFKNVDDFQDFLNSVLRRSLDEYRVFFENYERLFNSFSSKIFSMRTKSRLVVGLGDESVYETSIRLHRNYGVPYIPGSALKGVAKHYAFSILARENGDEILRIYESVKEDLKARIAKRDKIKKNDVPEDYYLTAAVIQELFEKKFDELGAIRNTRVEIGDTVISVGDIVKIFGTQKEEGSVIFFDAFPTPEQLKDKPNLELDIMNPHYQPYYQHGEPPGDWHSPNPIFFLTVPAGVEFTFAVASRDLDDLAEKAEKLLKEALKKFGVGAKTSLGYGRFDARDDR

SEQ ID NO: 180 - protein, 297 aa, Clostridium botulinum
MIGNIGYLYYKEYFKDEYIHECKIQSKKYDEKNNPLIKINQKILDNSNIDILKNNKFKFIKEIQDKLYKKRIYFKTTYPGLIVGTGYSHILKEKEEFKLSLEFDYTTGLPVINGSSIKGMLRSVFYNKKDDEKLIEEKEKYIRDILKELIKKENPKFNGEFDFEELTNNIFEGKCKAKDKNGIHMSISERDIFLGATIDIEATIEEMKRTKQEKNNLLGEDYVTPHGEGKDKLKNPNPIKFLKVMPNVVWCFGFDLKDFNKDIPADIKKKLFKQILLDLGIGAKTNVGYGRLEFISY